(19) United States
(12) Patent Application Publication
(10) Pub. No.: US 2004/0023874 A1
(43) Pub. Date: Feb. 5, 2004

(54) Therapeutic polypeptides, nucleic acids encoding same, and methods of use

(76) Inventors: Catherine E. Burgess, Wethersfield, CT (US); John S. Chant, Branford, CT (US); Ammitabha Chaudhuri, Madison, CT (US); Shlomit R. Edinger, New Haven, CT (US); Esha A. Gangolli, Acton, MA (US); Uriel M. Malyankar, Branford, CT (US); Charles E. Miller, Guilford, CT (US); Chean Eng Ooi, Branford, CT (US); Tatiana Ort, Milford, CT (US); Meera Patturajan, Branford, CT (US); Luca Rastelli, Guilford, CT (US); Daniel K. Rieger, Branford, CT (US); Richard A. Shimkets, Guilford, CT (US); Bryan D. Zerhusen, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/379,747

(22) Filed: Mar. 5, 2003

Related U.S. Application Data
(60) Provisional application No. 60/365,034, filed on Mar. 15, 2002.
(60) Provisional application No. 60/366,420, filed on Mar. 21, 2002.
(60) Provisional application No. 60/365,477, filed on Mar. 19, 2002.

(51) Int. Cl. G01N 33/53; A61K 38/17; C07K 14/47; C12P 21/02; C12N 1/21; C12N 1/16; C12N 1/18; C12N 5/06; C07H 21/04

(52) U.S. Cl. 514/12; 530/350; 536/23.5; 435/69.1; 435/320.1; 435/252.3; 435/254.2; 435/348; 435/325; 435/7.1

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. The invention further discloses therapeutic, diagnostic and research methods for diagnosis, treatment, and prevention of disorders involving any one of these novel human nucleic acids and proteins

### BACKGROUND OF THE INVENTION

Eukaryotic cells are characterized by biochemical and physiological processes, which under normal conditions are exquisitely balanced to achieve the preservation and propagation of the cells. When such cells are components of multicellular organisms such as vertebrates or, more particularly, organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example, two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including, by way of nonlimiting example, induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO: 2n, wherein n is an integer between 1 and 13. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO: 2n, wherein n is an integer between 1 and 13, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO: 2n, wherein n is an integer between 1 and 13. In another embodiment, the invention also comprises variants of the amino acid sequence selected from the group consisting of SEQ ID NO: 2n, wherein n is an integer between 1 and 13, wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO: 2n, wherein n is an integer between 1 and 13, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO: 2n, wherein n is an integer between 1 and 13. These allelic variants include amino acid sequences that are the translations of nucleic acid sequences differing by a single nucleotide from nucleic acid sequences selected from the group consisting of SEQ ID NOS: 2n−1, wherein n is an integer between 1 and 13. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO: 2n, wherein n is an integer between 1 and 13, and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO: 2n, wherein n is an integer between 1 and 13, wherein said therapeutic is the polypeptide selected from this group.

In another embodiment, the invention comprises a method for determining the presence or amount of a polypeptide with an amino acid sequence selected from the group consisting of SEQ ID NO: 2n, wherein n is an integer between 1 and 13, in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO: 2n, wherein n is an integer between 1 and 13, in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO: 2n, wherein n is an integer between 1 and 13, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO: 2n, wherein n is an integer between 1 and 13, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO: 2n, wherein n is an integer between 1 and 13, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO: 2n, wherein n is an integer between 1 and 13, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO: 2n, wherein n is an integer between 1 and 13, the method including administering the polypeptide to a subject in which such treatment or prevention is desired in an amount sufficient to treat or prevent the pathology in the subject The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO: 2n, wherein n is an integer between 1 and 13, or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO: 2n, wherein n is an integer between 1 and 13, a variant of a mature form of the amino acid sequence selected from the group consisting of SEQ ID NO: 2n, wherein n is an integer between 1 and 13, wherein any amino acid in the mature form of the chosen sequence is changed to a different amino acid, provided that no more than 15% of the amino acid residues in the sequence of the mature form are so changed; the amino acid sequence selected from the group consisting of SEQ ID NO: 2n, wherein n is an integer between 1 and 13, a variant of the amino acid sequence selected from the group consisting of SEQ ID NO: 2n, wherein n is an integer between 1 and 13, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO: 2n, wherein n is an integer between 1 and 13, or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO: 2n, wherein n is an integer between 1 and 13, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO: 2n, wherein n is an integer between 1 and 13, that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO: 2n, wherein n is an integer between 1 and 13, wherein the nucleic acid molecule differs by a single nucleotide from a nucleic acid sequence selected from the group consisting of SEQ ID NOS: 2n−1, wherein n is an integer between 1 and 13.

In another embodiment, the invention includes an isolated nucleic acid molecule having a nucleic acid sequence encoding a polypeptide including an amino acid sequence selected from the group consisting of a mature form of the amino acid sequence given SEQ ID NO: 2n, wherein n is an integer between 1 and 13, wherein the nucleic acid molecule comprises a nucleotide sequence selected from the group consisting of the nucleotide sequence selected from the group consisting of SEQ ID NO: 2n−1, wherein n is an integer between 1 and 13, a nucleotide sequence wherein one or more nucleotides in the nucleotide sequence selected from the group consisting of SEQ ID NO: 2n−1, wherein n is an integer between 1 and 13, is changed from that selected from the group consisting of the chosen sequence to a different nucleotide provided that no more than 15% of the nucleotides are so changed; a nucleic acid fragment of the sequence selected from the group consisting of SEQ ID NO: 2n−1, wherein n is an integer between 1 and 13, and a nucleic acid fragment wherein one or more nucleotides in the nucleotide sequence selected from the group consisting of SEQ ID NO: 2n−1, wherein n is an integer between 1 and 13, is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO: 2n, wherein n is an integer between 1 and 13, wherein the nucleic acid molecule hybridizes under stringent conditions to the nucleotide sequence selected from the group consisting of SEQ ID NO: 2n−1, wherein n is an integer between 1 and 13, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO: 2n, wherein n is an integer between 1 and 13, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO: 2n, wherein n is an integer between 1 and 13. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO: 2n, wherein n is an integer between 1 and 13, in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO: 2n, wherein n is an integer between 1 and 13, in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid) | acid) | Homology |
| NOV1a | CG103362-01 | 1 | 2 | Complement C3 precursor [Contains: |
| | | | | C3a anaphylatoxin] - Homo sapiens |
| NOV1b | CG103362-02 | 3 | 4 | Complement C3 precursor [Contains: |
| | | | | C3a anaphylatoxin] - Homo sapiens |
| NOV2a | CG127779-02 | 5 | 6 | Adiponectin precursor (30 kDa |
| | | | | adipocyte complement-related |
| | | | | protein) (ACRP30) (Adipose most |
| | | | | abundant gene transcript 1) (apM-1) |
| | | | | (Gelatin-binding protein) - Homo |
| | | | | sapiens |
| NOV2b | CG127779-01 | 7 | 8 | Adiponectin precursor (30 kDa |
| | | | | adipocyte complement-related |
| | | | | protein) (ACRP30) (Adipose most |
| | | | | abundant gene transcript 1) (apM-1) |
| | | | | (Gelatin-binding protein) - Homo |
| | | | | sapiens |
| NOV2c | CG127779-03 | 9 | 10 | Adiponectin precursor (30 kDa |
| | | | | adipocyte complement-related |
| | | | | protein) (ACRP30) (Adipose most |
| | | | | abundant gene transcript 1) (apM-1) |
| | | | | (Gelatin-binding protein) - Homo |
| | | | | sapiens |
| NOV2d | CG127779-04 | 11 | 12 | Adiponectin precursor (30 kDa |
| | | | | adipocyte complement-related |
| | | | | protein) (ACRP30) (Adipose most |
| | | | | abundant gene transcript 1) (apM-1) |
| | | | | (Gelatin-binding protein) - Homo |
| | | | | sapiens |
| NOV2e | CG127779-05 | 13 | 14 | Adiponectin precursor (30 kDa |
| | | | | adipocyte complement-related |
| | | | | protein) (ACRP30) (Adipose most |
| | | | | abundant gene transcript 1) (apM-1) |
| | | | | (Gelatin- binding protein) - Homo |
| | | | | sapiens |
| NOV3a | CG92035-03 | 15 | 16 | C1q-related factor precursor - Mus |
| | | | | musculus |
| NOV3b | CG92035-01 | 17 | 18 | C1q-related factor precursor - Mus |
| | | | | musculus |
| NOV3c | CG92035-02 | 19 | 20 | C1q-related factor precursor - Mus |
| | | | | musculus |
| NOV4a | CG169230-01 | 21 | 22 | Human calcium signal-modulating |
| | | | | cyclophilin ligand-like |
| NOV5a | CG169422-01 | 23 | 24 | Agouti switch protein precursor |
| | | | | (Agouti signaling protein) - Homo |
| | | | | sapiens |
| NOV6a | CG169440-01 | 25 | 26 | Agouti-related protein precursor — |
| | | | | Homo sapiens |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders, conditions and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 13, (b) a variant of a mature form of the amino acid sequence selected from the group consisting of SEQ ID NO: 2n, wherein n is an integer between 1 and 13, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 13, (d) a variant of the amino acid sequence selected from the group consisting of SEQ ID NO: 2n, wherein n is an integer between 1 and 13, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 13; (b) a variant of a mature form of the amino acid sequence selected from the group consisting of SEQ ID NO: 2n, wherein n is an integer between 1 and 13, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 13; (d) a variant of the amino acid sequence selected from the group consisting of SEQ ID NO: 2n, wherein n is an integer between 1 and 13, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 13, or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 13; (b) a nucleotide sequence wherein one or more nucleotides in the nucleotide sequence selected from the group consisting of SEQ ID NO: 2n−1, wherein n is an integer between 1 and 13, is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 13; and (d) a nucleic acid fragment wherein one or more nucleotides in the nucleotide sequence selected from the group consisting of SEQ ID NO: 2n−1, wherein n is an integer between 1 and 13, is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide, precursor form, or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a post-translational modification step other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, about 4 kb, about 3 kb, about 2 kb, about 1 kb, about 0.5 kb, or about 0.1 kb, of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NOS: 2n−1, wherein n is an integer between 1 and 13, or a complement of this nucleotide sequence, can be isolated using standard molecular biology techniques and the sequence information provided herein. Using all or a portion of the nucleic acid sequence of SEQ ID NOS: 2n−1, wherein n is an integer between 1 and 13, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993).

A nucleic acid of the invention can be amplified using cDNA, mRNA or, alternatively, genomic DNA as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 2n−1, wherein n is an integer between 1 and 13, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 2n−1, wherein n is an integer between 1 and 13, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NOS: 2n−1, wherein n is an integer between 1 and 13, is one that is sufficiently complementary to the nucleotide sequence of SEQ ID NOS: 2n−1, wherein n is an integer between 1 and 13, that it can hydrogen bond with few or no mismatches to a nucleotide sequence of SEQ ID NOS: 2n−1, wherein n is an integer between 1 and 13, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO: 2n−1, wherein n is an integer between 1 and 13, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 2n−1, wherein n is an integer between 1 and 13; or an anti-sense strand nucleotide sequence of SEQ ID NO: 2n−1, wherein n is an integer between 1 and 13; or of a naturally occurring mutant of SEQ ID NO: 2n−1, wherein n is an integer between 1 and 13.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO: 2n−1, wherein n is an integer between 1 and 13, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO: 2n−1, wherein n is an integer between 1 and 13, due to degeneracy of the genetic code and thus encode the same NOVX proteins as that encoded by the nucleotide sequences of SEQ ID NO: 2n−1, wherein n is an integer between 1 and 13. In another embodiment, an isolated nucleic acid molecule of the invention has a nucleotide sequence encoding a protein having an amino acid sequence of SEQ ID NO: 2n, wherein n is an integer between 1 and 13.

In addition to the human NOVX nucleotide sequences of SEQ ID NO: 2n−1, wherein n is an integer between 1 and 13, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO: 2n−1, wherein n is an integer between 1 and 13, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 2n−1, wherein n is an integer between 1 and 13. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO: 2n−1, wherein n is an integer between 1 and 13, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 2n−1, wherein n is an integer between 1 and 13, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO: 2n−1, wherein n is an integer between 1 and 13, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO: 2n−1, wherein n is an integer between 1 and 13, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO: 2n, wherein n is an integer between 1 and 13. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO: 2n−1, wherein n is an integer between 1 and 13, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO: 2n, wherein n is an integer between 1 and 13. Preferably, the protein encoded by the nucleic acid molecule is at least about 60% homologous to SEQ ID NO: 2n, wherein n is an integer between 1 and 13; more preferably at least about 70% homologous to SEQ ID NO: 2n, wherein n is an integer between 1 and 13; still more preferably at least about 80% homologous to SEQ ID NO: 2n, wherein n is an integer between 1 and 13; even more preferably at least about 90% homologous to SEQ ID NO: 2n, wherein n is an integer between 1 and 13; and most preferably at least about 95% homologous to SEQ ID NO: 2n, wherein n is an integer between 1 and 13.

An isolated nucleic acid molecule encoding a NOVX protein homologous to the protein of SEQ ID NO: 2n, wherein n is an integer between 1 and 13, can be created by introducing one or more nucleotide substitutions, additions or deletions into the nucleotide sequence of SEQ ID NO: 2n−1, wherein n is an integer between 1 and 13, such that one or more amino acid substitutions, additions or deletions are introduced into the encoded protein.

Mutations can be introduced any one of SEQ ID NO: 2n−1, wherein n is an integer between 1 and 13, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO: 2n−1, wherein n is an integer between 1 and 13, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2×proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n−1, wherein n is an integer between 1 and 13, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO: 2n, wherein n is an integer between 1 and 13, or antisense nucleic acids complementary to a NOVX nucleic acid sequence of SEQ ID NO: 2n−1, wherein n is an integer between 1 and 13, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO: 2n−1, wherein n is an integer between 1 and 13). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO: 2n, wherein n is an integer between 1 and 13. The invention also includes a mutant or variant protein any of whose residues may be changed from the corresponding residues shown in any one of SEQ ID NO: 2n, wherein n is an integer between 1 and 13, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO: 2n, wherein n is an integer between 1 and 13) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO: 2n, wherein n is an integer between 1 and 13. In other embodiments, the NOVX protein is substantially homologous to SEQ ID NO: 2n, wherein n is an integer between 1 and 13, and retains the functional activity of the protein of SEQ ID NO: 2n, wherein n is an integer between 1 and 13, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO: 2n, wherein n is an integer between 1 and 13, and retains the functional activity of the NOVX proteins of SEQ ID NO: 2n, wherein n is an integer between 1 and 13.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48:443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO: 2n−1, wherein n is an integer between 1 and 13.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO: 2n, wherein n is an integer between 1 and 13, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO: 2n, wherein n is an integer between 1 and 13, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Biospecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fe receptors for IgG (FcγR), such as FcγRI (CD64), FCγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fe regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington : The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can-be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NOS: 2n−1, wherein n is an integer between 1 and 13, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NOS: 2n−1, wherein n is an integer between 1 and 13), but more preferably, is a non-human homologue of a human NOVX gene. For example, a mouse homologue of human NOVX gene of SEQ ID NOS: 2n−1, wherein n is an integer between 1 and 13, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987 In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. a NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be deter-mined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of a NOVX sequence, i.e., of SEQ ID NOS: 2n−1, wherein n is an integer between 1 and 13, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NOS: 2n−1, wherein n is an integer between 1 and 13, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials. These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 2n−1, wherein n is an integer between 1 and 13, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome Pregnancy Zone Protein Precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

Polynucleotide And Polypeptide Sequences, And Homology Data

### Example 1

The NOV1 clone was analyzed, and the nucleotide and encoded polypeptide sequences are shown in Table 1A.

TABLE 1A
NOV1 Sequence Analysis

| | SEQ ID NO:1 | 5067 bp |
|---|---|---|
| NOV 1a, | CTCCTCCCCATCCTCTCCCTCTGTCCCTCTGTCCCTCTGACCCTGCACTGTCCCAGCA | |
| CG103362-01 | CCATGGGACCCACCTCAGGTCCCACCCTGCTGCTCCTGCTACTAACCCACCTCCCCCT | |
| DNA Sequence | GGCTCTGGCGAGTCCCATGTACTCTATCATCACCCCCAACATCTTGCGGCTGGAGAGC | |
| | GAGGAGACCATGGTGCTCGAGGCCCACGACGCGCAAGGGGATGTTCCAGTCACTGTTA | |
| | CTGTCGACGACTTCCCAGGCAAAAAACTAGTGCTGTCCAGTGAGAAGACTGTGCTGAC | |
| | CCCTGCCACCAACCACATGGGCAACGTCACCTTCACGATCCCAGCCAACAGGGAGTTC | |
| | AAGTCAGAAAAGGGGCGCAACAAGTTCGTGACCGTGCAGGCCACCTTCGGGACCCAAG | |
| | TGGTGGAGAAGGTGGTGCTGGTCAGCCTGCAGAGCGGGTACCTCTTCATCCAGACAGA | |
| | CAAGACCATCTACACCCCTGGCTCCACAGTTCTCTATCGGATCTTCACCGTCAACCAC | |
| | AAGCTGCTACCCGTGGGCCGGACGGTCATGGTCAACATTGAGAACCCGGAAGGCATCC | |
| | CGGTCAAGCAGGACTCCTTGTCTTCTCAGAACCAGCTTGGCGTCTTGCCCTTGTCTTG | |
| | GGACATTCCGGAACTCGTCAACATGGGCCAGTGGAAGATCCGAGCCTACTATGAAAAC | |
| | TCACCACAGCAGGTCTTCTCCACTGAGTTTGAGGTGAAGGAGTACGTGCTGCCCAGTT | |
| | TCGAGGTCATAGTGGAGCCTACAGAGAAATTCTACTACATCTATAACGAGAAGGGCCT | |
| | GGAGGTCACCATCACCGCCAGGTTCCTCTACGGGAAGAAAGTGGAGGGAACTGCCTTT | |
| | GTCATCTTCGGGATCCAGGATGCGAACAGAGGATTTCCCTGCCTGAATCCCTCAAAGC | |
| | GCATTCCGATTGAGGATGGCTCGGGGGAGGTTGTGCTGAGCCGGAAGGTACTGCTGGA | |
| | CGGGGTGCAGAACCTCCGAGCAGAAGACCTGGTGGGGAAGTCTTTGTACGTGTCTGCC | |
| | ACCGTCATCTTGCACTCAGGCAGTGACATGGTGCAGGCAGAGCGCAGCGGGATCCCCA | |
| | TCGTCACCTCTCCCTACCAGATCCACTTCACCAAGACACCCAAGTACTTCAAACCAGG | |
| | AATGCCCTTTGACCTCATGGTGTTCGTGACGAACGCTGATGGCTCTCCAGCCTACCGA | |
| | GTCCCCGTGGCAGTCCAGGGCGAGGACACTGTGCAGTCTCTAACCCAGGGAGATGGCG | |
| | TGGCCAAACTCAGCATCAACACACACCCCAGCCAGAAGCCCTTGAGCATCACGGTGCG | |
| | CACGAACAAGCAGGAGCTCTCGGAGGCAGAGCAGGCTACCAGGACCATGCAGGCTCTG | |
| | CCCTACAGCACCGTGGGCAACTCCAACAATTACCTGCATCTCTCAGTGCTACGTACAG | |
| | AGCTCAGACCCGGCGAGACCCTCAACGTCAACTTCCTCCTGCGAATGGACCGCGCCCA | |
| | CGAGGCCAAGATCCGCTACTACACCTACCTGATCATGAACAAGGGCAGGCTGTTGAAG | |
| | GCGGGACGCCAGGTGCGAGACCCCGGCCAGGACCTGGTCGTGCTGCCCCTCTCCATCA | |
| | CCACCGACTTCATCCCTTCCTTCCGCCTGGTGGCGTACTACACGCTGATCGGTGCCAG | |
| | CGGCCAGAGGGAGGTGGTGGCCGACTCCGTGTGGGTGGACGTCAAGGACTCCTGCGTG | |
| | GGCTCGCTGGTGGTAAAAAGCGGCCAGTCAGAAGACCGGCAGCCTGTACCTGGGCAGC | |
| | AGATGACCCTGAAGATAGAGGGTGACCACGGGGCCCGGGTGGTACTGGTGGCCGTGGA | |
| | CAAGGGCGTGTTCGTGCTGAATAAGAAGAACAAACTGACGCAGAGTAAGATCTGGGAC | |
| | GTGGTGGAGAACGCAGACATCGGCTGCACCCCGGGCAGTGGGAAGGATTACGCCGGTG | |
| | TCTTCTCCGACGCAGGGCTGACCTTCACGAGCAGCAGTGGCCAGCACACCGCCCAGAC | |
| | GCCAGAACTTCAGTGCCCGCAGCCAGCCGCCCGCCGACGCCGTTCCGTGCAGCTCACG | |
| | GAGAAGCGAATGGACAAAGTCGGCAAGTACCCCAAGGAGCTGCGCAAGTGCTGCGAGG | |
| | ACGGCATGCGGGAGAACCCCATGAGGTTCTCGTGCCAGCGCCGGACCCGTTTCATCTC | |
| | CCTGGGCGAGGCGTGCAAGAAGGTCTTCCTGGACTGCTGCAACTACATCACAGAGCTG | |
| | CGGCGGCAGCACGCGCGGGCCAGCCACCTGGGCCTGGCCAGGAGTAACCTGGATGAGG | |
| | ACATCATTGCAGAAGAGAACATCGTTTCCCGAAGTGAGTTCCCAGAGAGCTGGCTGTG | |
| | GAACGTTGAGGACTTGAAAGAGCCACCGAAAAATGGAATCTCTACGAAGCTCATGAAT | |
| | ATATTTTTGAAAGACTCCATCACCACGTGGGAGATTCTGGCTGTCAGCATGTCGGACA | |
| | AGAAAGGGATCTGTGTGGCAGACCCCTTCGAGGTCACAGTAATGCAGGACTTCTTCAT | |
| | CGACCTGCGGCTACCCTACTCTGTTGTTCGAAACGAGCAGGTGGAAATCCGAGCCGTT | |
| | CTCTACAATTACCGGCAGAACCAAGAGCTCAAGGTGAGGGTGGAACTACTCCACAATC | |
| | CAGCCTTCTGCAGCCTGGCCACCACCAAGAGGCGTCACCAGCAGACCGTAACCATCCC | |
| | CCCCAAGTCCTCGTTGTCCGTTCCATATGTCATCGTGCCGCTAAAGACCGGCCTGCAG | |
| | GAAGTGGAAGTCAAGGCTGCCGTCTACCATCATTTCATCAGTGACGGTGTCAGGAAGT | |
| | CCCTGAAGGTCGTGCCGGAAGGAATCAGAATGAACAAAACTGTGGCTGTTCGCACCCT | |
| | GGATCCAGAACGCCTGGGCCGTGAAGGAGTGCAGAAAGAGGACATCCCACCTGCAGAC | |
| | CTCAGTGACCAAGTCCCGGACACCGAGTCTGAGACCAGAATTCTCCTGCAAGGGACCC | |
| | CAGTGGCCCAGATGACAGAGGATGCCGTCGACGCGCAACGGCTGAAGCACCTCATTGT | |
| | GACCCCCTCGGGCTGCGGGGAACACAACATGATCGGCATGACGCCCACGGTCATCGCT | |
| | GTGCATTACCTGGATGAAACGGAGCAGTGGGAGAAGTTCCGCCTAGAGAAGCGGCAGG | |
| | GGGCCTTGGAGCTCATCAAGAAGGGGTACACCCAGCAGCTGGCCTTCAGACAACCCAG | |
| | CTCTGCCTTTGCGGCCTTCGTGAAACGGGCACCCAGCACCTGGCTGACCGCCTACGTG | |
| | GTCAACGTCTTCTCTCTGGCTGTCAACCTCATCGCCATCGACTCCCAAGTCCTCTGCG | |
| | GGGCTGTTAAATGGCTCATCCTGGAGAAGCAGAAGCCCGACGGGGTCTTCCAGGACGA | |
| | TGCGCCCCTGATACACCAAGAAATGATTGGTGGATTACGGAACAACAACGAGAAAGAC | |
| | ATGGCCCTCACCGCCTTTGTTCTCATCTCGCTGCAGGAGGCTAAAGATATTTGCGAGG | |
| | AGCAGGTCAACAGCCTGCCAGGCAGCATCACTAAAGCAGCAGACTTCCTTGAAGCCAA | |
| | CTACATGAACCTACAGAGATCCTACACTGTGGCCATTGCTGGCTATGCTCTCGCCCAG | |
| | ATGGGCAGCCTGAAGGGGCCTCTTCTTAACAAATTTCTGACCACAGCCAAAGATAAGA | |
| | ACCGCTGGGAGGACCCTGGTAAGCAGCTCTACAACGTGOAGGCCACATCCTATGCCCT | |
| | CTTGGCCCTACTGCAGCTAAAAGACTTTGACTTTGTGCCTCCCGTCGTGCGTTGGCTC | |
| | AATGAACAGAGATACTACGGTGGTGOCTATGGCTCTACCCAGGCCACCTTCATGGTGT | |
| | TCCAAGCCTTGGCTCAATACCAAAAGGACGCCCCTGACCACCAGGAACTGAACCTTGA | |
| | TGTGTCCCTCCAACTGCCCAGCCGCAGCTCCAAGATCACCCACCGTATCCACTGGGAA | |
| | TCTGCCAGCCTCCTGCGATCAGAAGAGACCAAGGAAAATGAGGGTTTCACAGTCACAG | |
| | CTGAAGGAAAAGGCCAAGGCACCTTGTCGGTGGTCACAATGTACCATGCTAAGGCCAA | |
| | AGATCAACTCACCTGTAATAAATTCGACCTCAAGGTCACCATAAAACCAGCACCGGAA | |
| | ACAGAAAAGAGGCCTCAGGATGCCAAGAACACTATGATCCTTGAGATCTGTACCAGGT | |
| | ACCCGGGAGACCAGGATGCCACTATGTCTATATTGGACATATCCATGATGACTGGCTT | |
| | TGCTCCACACACAGATGACCTGAAGCAGCTGCCCAATGGTGTTGACAGATACATCTCC | |
| | AAGTATGAGCTGGACAAAGCCTTCTCCGATAGGAACACCCTCATCATCTACCTGGACA | |
| | AGGTCTCACACTCTGAGGATGACTGTCTAGCTTTCAAAGTTCACCAATACTTTAATGT | |
| | AGAGCTTATCCAGCCTGGAGCAGTCAAGGTCTACGCCTATTACAACCTGGAGGAAAGC | |
| | TGTACCCCGTTCTACCATCCGGAAAAGGAGGATGGAAAGCTGAACAAGCTCTGCCGTG | |
| | ATGAACTGTGCCGCTGTGCTGAGGAGAATTGCTTCATACAAAAGTCGGATGACAAGGT | |
| | CACCCTGGAAGAACGGCTGGACAAGGCCTGTCAGCCAGGAGTGGACTATGTGTACAAG | |
| | ACCCGACTGGTCAAGGTTCAGCTGTCCAATGACTTTGACGAGTACATCATGGCCATTG | |
| | AGCAGACCATCAAGTCAGGCTCGGATGAGGTGCAGGTTGGACAGCAGCGCACGTTCAT | |
| | CAGCCCCATCAAGTGCAGAGAAGCCCTGAAGCTGGAGGAGAAGAAACACTACCTCATG | |
| | TGGGGTCTCTCCTCCGATTTCTGGGGAGAGAAGCCCAACCTCAGCTACATCATCGGGA | |
| | AGGACACTTGGGTGGAGCACTGGCCTGACGAGGACGATGCCAGACGAAGAGAACCA | |
| | GAAACAATGCCAGGACCTCGGCGCCTTCACCGAGAGCATGGTTGTCTTTGGGTGCCCC | |
| | AACTGACCACACCCCCATTCC | |
| | ORF Start: ATG at 61 | ORF Stop: TGA at 5050 |
| | SEQ ID NO:2 | 1663 aa MW at 187162.3 kD |
| NOV1a, | MGPTSGPSLLLLLLTHLPLALGSPMYSIITPNILRLESEETMVLEAHDAQGDVPVTVT | |
| CG103362-01 | VHDFPGKKLVLSSEKTVLTPATNHMCNVTFTIPANREFKSEKGRNKFVTVQATFGTQV | |
| Protein Sequence | VEKVVLVSLQSGYLFIQTDKTIYTPGSTVLYRIFTVNHKLLPVGRTVMVNIENPEGIP | |
| | VKQDSLSSQNQLGVLPLSWDIPELVNMGQWKIRAYYENSPQQVFSTEFEVKEYVLPSF | |
| | EVIVEPTEKFYYIYNEKGLEVTITARFLYGKKVEGTAFVIFGTQDCEQRISLPESLKR | |
| | IPIEDGSGEVVLSRKVLLDGVQNLRAEDLVGKSLYVSATVILHSGSDMVQAERSGIPI | |
| | VTSPYQIHFTKTPKYFKPGMPFDLMVFVThPDGSPAYRVPVAVQGEDTVQSLTQGDGV | |
| | AKLSINTHPSQKPLSITVRTKKQELSEAEQATRTMQALPYSTVGNSNNYLHLSVLRTE | |
| | LRPGETLNVNFLLRMDRAHEAKIRYYTYLIMNKGRLLKAGRQVREPGQDLVVLPLSIT | |
| | TDFIPSFRLVAYYTLIGASGQREVVADSVWVDVKDSCVGSLVVKSGQSEDRQPVPGQQ | |
| | MTLKIEGDHGARVVLVAVDKGVFVLNKKNKLTQSKIWDVVEKADICCTPGSGKDYAGV | |
| | FSDAGLTFTSSSOQQTAQRAELQCPQPAARRRRSVQLTEKRMDKVGKYPKELRKCCED | |
| | GMRENPMRFSCQRRTRFISLGEACKKVFLDCCNYITELRRQHARASHLGLARSNLDED | |
| | IIAEENIVSRSEFPESWLWNVEDLKEPPKNGISTKLMNIFLKDSITTWEILAVSMSDK | |
| | KGICVADPFEVTVMQDFFIDLRLPYSVVRNEQVEIHAVLYNYRQNQELKVRVELLHNP | |
| | AFCSLATTKRRHQQTVTIPPKSSLSVPYVIVPLKTGLQEVEVKAAVYHHFISDGVRKS | |
| | LKVVPEGIRMNKTVAVRTLDPERLGREGVQKEDIPPADLSDQVPDTESETRILLQGTP | |
| | VAQMTEDAVDAERLKHLIVTPSGCGEQNMIGMTPTVIAVHYLDETEQWEKFGLEKRQG | |
| | ALELIKKGYTQQLAFRQPSSAFAAFVKRAPSTWLTAYVVKVFSLAVNLIAIDSQVLCG | |
| | AVKWLILEKQKPDGVFQEDAPVIHQEMIGGLRNNNEKDMALTAFVLISLQEAKDICEE | |
| | QVNSLPGSITKAGDFLEANYMNLQRSYTVAIAGYALAQMORLKGPLLNKFLTTAKDKN | |
| | RWEDPGKQLYNVEATSYALLALLQLKDFDFVPPVVRWLNEQRYYGGGYGSTQATFMVP | |
| | QALAQYQKDAPDHQELNLDVSLQLPSRSSKITHRIHWESASLLRSEETKENEGFTVTA | |
| | EGKGQGTLSVVTMYHAKAKDQLTCNKFDLKVTIKPAPETEKRPQDAKNTMILEICTRY | |
| | RGDQDATMSILDISMMTGFAPDTDDLKQLANGVDRYISKYELDKAFSDRNTLIIYLDK | |
| | VSHSEDDCLAFKVHQYFNVELIQPGAVKVYAYYNLEESCTRFYHPEKEDGKLNKLCRD | |
| | ELCRCAEENCFIQKSDDKVTLEERLDKACEPGVDYVYKTRLVKVQLSNDFDEYIMAIE | |
| | QTIKSGSDEVQVGQQRTFISPIKCREALKLEEKKHYLMWGLSSDFWGEKPNLSYIIGK | |
| | DTWVEHWPEEDECQDEENQKQCQDLGAFTESMVVFGCPN | |
| | SEQ ID NO:3 | 2475 bp |
| NOV 1b, | GCTGGGTGTTTGAGGGTGTGATGTGTGTGCATGTGTCTGGCCGTGGGACACGCCGTGC | |
| CG103362-02 | GAAGGCGTGGGCATATGAGACTGTGTCAGCCCGTGTCATCTGGGATGCCTCATGTGTG | |
| DNA Sequence | CAGCTGGGTGTGTCTGTGTGAGCACGGCAGAGTGGGGGGCTCACAGCACACAGGCGTC | |
| | TGCTGCAGAAAAGAGTCCGGCGGTCCATCATCCTAGGTCACATCTGCATCACCTTGTC | |
| | ACTATCTTGTTTCCTCTTACTCCTCCCCATCCTCTCCCTCTGTCCCTCTGTCCCTCTG | |
| | ACCCTGCACTGTCCCAGCACCATGGGACCCACCTCAGGTCCCAGCCTGCTGCTCCTGC | |
| | TACTAACCCACCTCCCCCTGGCTCTGGGGAGTCCCATGTACTCTATCATCACCCCCAA | |
| | CATCTTGCGGCTGGAGAGCGAGGAGACCATGGTGCTGGAGGCCCACGACGCOCAAGGG | |
| | GATGTTCCAGTCACTGTTACTGTCCACGACTTCCCAGGCAAAAAACTAGTGCTGTCCA | |
| | GTGAGAAGACTGTGCTGACCCCTGCCACCAACCACATGGGCAACGGGGCCTTGGAGCT | |
| | CATCAAGAAGGGGTACACCCAGCAGCTGGCCTTCAGACAACCCAGCTCTGCCTTTGCG | |
| | GCCTTCGTGAAACCGGCACCCAGCACCTGGCTCACCGCCTACGTGGTCAAGGTCTTCT | |
| | CTCTGGCTGTCAACCTCATCGCCATCOACTCCCAAGTCCTCTGCGGGGCTGTTAAATG | |
| | GCTGATCCTGGAGAAGCAGAAGCCCGACGGGGTCTTCCAGGAGGATGCGCCCGTGATA | |
| | CACCAAGAAATGATTGGTGGATTACGGAACAACAACGAGAAAGACATGGCCCTCACGG | |
| | CCTTTGTTCTCATCTCGCTGCAGGAGGCTAAAGATATTTGCGAGGAGCAGGTCAACAG | |
| | CCTGCCACGCAGCATCACTAAAGCAGGAGACTTCCTTGAAGCCAACTACATGAACCTA | |
| | CAGAGATCCTACACTGTGGCCATTGCTGGCTATGCTCTGGCCCAGATCGGCAGGCTGA | |
| | AGGGGCCTCTTCTTAACAAATTTCTGACCACAGCCAAAGATAAGAACCGCTGGGAGGA | |
| | CCCTGGTAAGCAGCTCTACAACGTGGAGGCCACATCCTATGCCCTCTTGGCCCTACTG | |
| | CAGCTAAAAGACTTTGACTTTGTGCCTCCCGTCGTGCGTTGGCTCAATGAACAGAGAT | |
| | ACTACGGTGGTGGCTATGGCTCTACCCAGGCCACCTTCATGGTGTTCCAAGCCTTGGC | |
| | TCAATACCAAAAGGACGCCCCTGACCACCAGCAACTGAACCTTGATGTGTCCCTCCAA | |
| | CTGCCCAGCCGCAGCTCCAAGATCACCCACCGTATCCACTGGGAATCTGCCAGCCTCC | |
| | TGCGATCAGAAGAGACCAAGGAAAATGAGGGTTTCACAGTCACACCTGAAGCAAAAGG | |
| | CCAAGGCACCTTGTCGGTGGTGACAATGTACCATGCTAAGGCCAAAGATCAACTCACC | |
| | TGTAATAAATTCGACCTCAAGGTCACCATAAAACCAGCACCGGAAACAGAAAAGAGGC | |
| | CTCAGGATGCCAAGAACACTATGATCCTTGAGATCTGTACCAGGTACCGGGGAGACCA | |
| | GGATGCCAGTATGTCTATATTGGACATATCCATGATGACTGGCTTTGCTCCAGACACA | |
| | GATCACCTGAAGCAGCTGGCCAATGGTGTTGACAGATACATCTCCAAGTATGAGCTGG | |
| | ACAAAGCCTTCTCCGATAGGAACACCCTCATCATCTACCTGGACAAGGTCTCACACTC | |
| | TGAGGATGACTGTCTAGCTTTCAAAGTTCACCAATACTTTAATGTAGAGCTTATCCAG | |
| | CCTGGAGCAGTCAAGGTCTACGCCTATTACAACCTGGAGGAAAGCTGTACCCGGTTCT | |
| | ACCATCCGGAAAAGGACGATGGAAAGCTGAACAAGCTCTGCCGTGATCAACTGTGCCG | |
| | CTGTGCTGAGGACAATTGCTTCATACAAAAGTCGGATGACAAGGTCACCCTGGAAGAA | |
| | CGGCTGGACAAGGCCTGTGAGCCACGAGTGGACTATGTOTACAAGACCCGACTGGTCA | |
| | AGGTTCAGCTGTCCAATCACTTTGACGAGTACATCATGGCCATTGAGCAGACCATCAA | |
| | GTCAGGCTCGGATGACGTGCAGGTTGGACAGCAGCGCACGTTCATCAGCCCCATCAAC | |
| | TGCAGACAAGCCCTGAAGCTGGAGGAGAAGAAACACTACCTCATGTGGGGTCTCTCCT | |
| | CCGATTTCTCGGGAGAGAAGCCCAACCTCAGCTACATCATCGGGAAGCACACTTGGGT | |
| | GGAGCACTGGCCCGAGGAGGACGAATGCCAACACGAAGAGAACCAGAAACAATGCCAG | |
| | GACCTCGGCGCCTTCACCGAGAGCATGGTTGTCTTTGGGTGCCCCAACTGACCACACC | |
| | CCCATTCCCCCACTCCAGATAAAGCTTCAGTTATATCTC | |
| | ORF Start: ATG at 312 | ORF Stop: TGA at 2427 |
| | SEQ ID NO:4 | 705 aa MW at 79529.8 kD |

| | |
|---|---|
| NOV1b, | MGPTSGPSLLLLLLTHLPLALGSPMYSIITPNILRLESEETMVLEAHDAQGDVPVTVT |
| CG103362-02 | VHDFPGKKLVLSSEKTVLTPATNHMGNGALELIKKGYTQQLAFRQPSSAFAAFVKRAP |
| Protein Sequence | STWLTAYVVKVFSLAVNLIAIDSQVLCGAVKWLILEKQKPDGVFQEDAPVIHQEMIGG |
| | LRNNNEKDMALTAPVLISLQEAKDICEEQVNSLPGSITKAGDFLEANYMNLQRSYTVA |
| | IAGYALAQMGRLKGPLLNKFLTTAKDKNRWEDPGKQLYNVEATSYALLALLQLKDFDF |
| | VPPVVRWLNEQRYYGGGYCSTQATFMVFQALAQYQKDAPDHQELNLDVSLQLPSRSSK |
| | ITHRIHWESASLLRSEETKENEGFTVTAEGKGQGTLSVVTMYHAKAKDQLTCNKFDLK |
| | VTIKPAPETEKRPQDAKNTMILEICTRYRGDQDATMSILDISMMTGFAPDTDDLKQLA |
| | NGVDRYISKYELDKAFSDRNTLIIYLDKVSHSEDDCLAFKVHQYFNVELIQPGAVKVY |
| | AYYNLEESCTRFYHPEKEDGKLNKLCRDELCRCAEENCFIQKSDDKVTLEERLDKACE |
| | PGVDYVYKTRLVKVQLSNDFDEYIMAIEQTIKSGSDEVQVGQQRTFISPIKCREALKL |
| | EEKKHYLMWGLSSDFWGEKPNLSYIIGKDTWVEHWPEEDECQDEENQKQCQDLGAFTE |
| | SMVVFGCPN |

Sequence comparison of the above protein sequences yields the following sequence relationships shown in Table 1B.

TABLE 1B
Comparison of NOV1a against NOV1b.

| | NOV1a Residues/ | Identities/Similarities |
|---|---|---|
| Protein Sequence | Match Residues | for the Matched Region |
| NOV1b | 1044 . . . 1663 | 620/620 (100%) |
| | 86 . . . 705 | 620/620 (100%) |

Further analysis of the NOV1a protein yielded the following properties shown in Table 1C.

TABLE 1C
Protein Sequence Properties NOV1a

| SignalP analysis: | Cleavage site between residues 23 and 24 | |
|---|---|---|
| PSORT II analysis: | PSG: a new signal peptide prediction method | |
| | N-region: length 0; pos.chg 0; neg.chg 0 | |
| | H-region: length 34; peak value 9.71 | |
| | PSG score: 5.31 | |
| | GvH: von Heijne's method for signal seq. recognition | |
| | GvH score (threshold: −2.1): 7.83 | |
| | possible cleavage site: between 22 and 23 | |
| | >>> Seems to have a cleavable signal peptide (1 to 22) | |
| | ALOM: Klein et al's method for TM region allocation | |
| | Init position for calculation: 23 | |
| | Tentative number of TMS(s) for the threshold 0.5: 1 | |
| | Number of TMS(s) for threshold 0.5: 1 | |
| | INTEGRAL Likelihood = −2.39 Transmembrane 1079-1095 | |
| | PERIPHERAL Likelihood = 0.74 (at 116) | |
| | ALOM score: −2.39 (number of TMSs: 1) | |
| | MTOP: Prediction of membrane topology (Hartmann et al.) | |
| | Center position for calculation: 11 | |
| | Charge difference: −0.5 C(0.5) − N(1.0) | |
| | N >= C: N-terminal side will be inside | |
| | >>> membrane topology: type 1a (cytoplasmic tail 1096 to 1663) | |
| | MITDISC: discrimination of mitochondrial targeting seq | |
| | R content: 1 | Hyd Moment(75): 1.92 |
| | Hyd Moment(95): 2.35 | G content: 3 |
| | D/E content: 1 | S/T content: 7 |
| | Score: −4.42 | |

Gavel: prediction of cleavage sites for mitochondrial preseq
R-2 motif at 45 LRL|ES
NUCDISC: discrimination of nuclear localization signals
pat4: RRRR (5) at 668
pat4: KRRH (3) at 879
pat7: PAARRRR (5) at 665
bipartite: none
content of basic residues: 11.7%
NLS Score: 0.46
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 1206
LL at 1237
LL at 1240
LL at 1318
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupa's algorithm to detect coiled-coil regions
total: 0 residues

Final Results (k = 9/23):
44.4%: endoplasmic reticulum
22.2%: Golgi
22.2%: extracellular, including cell wall
11.1%: plasma membrane
>> prediction for CG103362-01 is end (k = 9)

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW34606 | Wild type human C3 protein - | 1 . . . 1663 | 1661/1663 (99%) | 0.0 |
| | Homo sapiens, 1663 aa. | 1 . . . 1663 | 1662/1663 (99%) | |
| | [WO9732981-A1, 12-SEP-1997] | | | |
| AAW34616 | Human C3 protein mutant CV-5 - | 1 . . . 1663 | 1660/1663 (99%) | 0.0 |
| | Homo sapiens, 1663 aa. | 1 . . . 1663 | 1662/1663 (99%) | |
| | [WO9732981-A1, 12-SEP-1997] | | | |
| AAW34610 | Human C3 protein mutant CV-2 - | 1 . . . 1663 | 1660/1663 (99%) | 0.0 |
| | Homo sapiens, 1663 aa. | 1 . . . 1663 | 1662/1663 (99%) | |
| | [WO9732981-A1, 12-SEP-1997] | | | |
| AAW34609 | Human C3 protein mutant C3M-51 - | 1 . . . 1663 | 1660/1663 (99%) | 0.0 |
| | Homo sapiens, 1663 aa. | 1 . . . 1663 | 1662/1663 (99%) | |
| | [WO9732981-A1, 12-SEP-1997] | | | |
| AAW34607 | Human C3 protein mutant C3M-I23 - | 1 . . . 1663 | 1660/1663 (99%) | 0.0 |
| | Homo sapiens, 1663 aa. | 1 . . . 1663 | 1662/1663 (99%) | |
| | [WO9732981-A1, 12-SEP-1997] | | | |

In a BLAST search of public sequence datbases, the NOV1a protein was found to have homology to the proteins shown in the BLASTP data in Table 1E.

TABLE 1E
Public BLASTP Results for NOV1a

| | | NOV1a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P01024 | Complement C3 precursor | 1 . . . 1663 | 1663/1663 (100%) | 0.0 |
| | [Contains: C3a anaphylatoxin] - | 1 . . . 1663 | 1663/1663 (100%) | |
| | Homo sapiens (Human), 1663 | | | |
| | aa. | | | |
| P01026 | Complement C3 precursor | 1 . . . 1663 | 1300/1666 (78%) | 0.0 |
| | [Contains: C3A anaphylatoxin] - | 1 . . . 1663 | 1472/1666 (88%) | |
| | Rattus norvegicus (Rat), 1663 | | | |
| | aa. | | | |
| P12387 | Complement C3 precursor | 1 . . . 1663 | 1298/1670 (77%) | 0.0 |
| | [Contains: C3A anaphylatoxin] - | 1 . . . 1666 | 1477/1670 (87%) | |
| | Cavia porcellus (Guinea pig), | | | |
| | 1666 aa. | | | |
| AAH43338 | Similar to complement | 1 . . . 1663 | 1283/1666 (77%) | 0.0 |
| | component 3 - Mus musculus | 1 . . . 1663 | 1463/1666 (87%) | |
| | (Mouse), 1663 aa. | | | |
| P01027 | Complement C3 precursor | 1 . . . 1663 | 1283/1666 (77%) | 0.0 |
| | (HSE-MSF) [Contains: C3A | 1 . . . 1663 | 1463/1666 (87%) | |
| | anaphylatoxin] - Mus musculus | | | |
| | (Mouse), 1663 aa. | | | |

PFam analysis predicts that the NOV1a protein contains the domains shown in the Table 1F.

TABLE 1F
Domain Analysis of NOV1a

| | | Identities/ | |
|---|---|---|---|
| | NOV1a | Similarities | Expect |
| Pfam Domain | Match Region | for the Matched Region | Value |
| A2M_N | 1 . . . 626 | 309/678 (46%) | 0 |
| | | 613/678 (90%) | |
| ANATO | 693 . . . 728 | 22/37 (59%) | 2.4e−18 |
| | | 35/37 (95%) | |
| A2M | 756 . . . 1493 | 377/847 (45%) | 0 |
| | | 726/847 (86%) | |
| NTR | 1533 . . . 1644 | 56/131 (43%) | 1.9e−54 |
| | | 107/131 (82%) | |

### Example 2

The NOV2 clone was analyzed, and the nucleotide and encoded polypeptide sequences are shown in Table 2A.

TABLE 2A
NOV2 Sequence Analysis

| | SEQ ID NO:5 | 783 bp |
|---|---|---|
| NOV2a, | CAGGGCTCAGGATGCTGTTGCTGGGAGCTGTTCTACTGCTATTAGCTCTGCCCGGTCA | |
| CG12779-02 | TGACCAGGAAACCACGACTCAAGGGCCCGGAGTCCTGCTTCCCCTGCCCAAGGGGGCC | |
| DNA Sequence | TGCACAGGTTGGATGGCGGGCATCCCAGCGCATCCGGGCCATAATGGGGCCCCAGGCC | |
| | GTGATGGCAGAGATGGCACCCCTOCTGAGAAGGGTGAGAAAGGAGATCCAGGTCTTAT | |
| | TGGTCCTAAGGGAGACATCGGTCAAACCGGAGTACCCGGGGCTGAAGGTCCCCGAGGC | |
| | TTTCCGCGAATCCAAGGCAGGAAAGGAGAACCTGCAGAAGGTGCCTATGTATACCGCT | |
| | CAGCATTCAGTGTOGGATTGGAGACTTACGTTACTATCCCCAACATGCCCATTCGCTT | |
| | TACCAAGATCTTCTACAATCAGCAAAACCACTATGATGGCTCCACTGGTAAATTCCAC | |
| | TGCAACATTCCTGGGCTGTACTACTTTGCCTACCACATCACAGTCTATATGAAGGATG | |
| | TGAACGTCAGCCTCTTCAAGAAGGACAAGGCTATGCTCTTCACCTATGATCAGTACCA | |
| | GGAAAATAATCTGGACCAGGCCTCCGGCTCTGTGCTCCTGCATCTGGAGGTGGGCGAC | |
| | CAAGTCTGGCTCCAGGTGTATGGGCAAGGAGAGCGTAATGGACTCTATGCTGATAATG | |
| | ACAATGACTCCACCTTCACAGGCTTTCTTCTCTACCATGACACCAACTGATCACCACT | |
| | AACTCAGAGCCTCCTCCAGGCCAAACAGC | |
| | ORF Start: ATG at 12 | ORF Stop: TGA at 744 |
| | SEQ ID NO:6 | 244 aa MW at 26413.5 kD |
| NOV2a, | MLLLGAVLLLLALPGHDQETTTQGPGVLLPLPKGACTCWMAGIPGHPGHNGAPGRDGR | |
| CG12779-02 | DGTPGEKGEKGDPCLIGPKGDIGETGVPGAECPRGFPGIQGRKGEPGEGAYVYRSAFS | |
| Protein Sequence | VGLETYVTIPNMPIRFTKIFYNQQNHYDGSTCKFHCNIPGLYYFAYHITVYMKDVKVS | |
| | LFKKDKAMLFTYDQYQENNVDQASCSVLLHLEVGDQVWLQVYGEGERNGLYADNDNDS | |
| | TFTGFLLYHDTN | |
| | SEQ ID NO:7 | 4545 bp |
| NOV2b, | ATTCTGACTGCAGTCTGTGGTTCTGATTCCATACCAGAGGGGCTCAGGATGCTGTTGC | |
| CG127779-01 | TGGGAGCTGTTCTACTGCTATTAGCTCTGCCCGGNCATGACCAGGAAACCACGACTCA | |
| DNA Sequence | AGGGCCCGGAGTCCTGCTTCCCCTGCCCAAGGGGGCCTGCACAGGTTGGATGGCGGGC | |
| | ATCCCAGGGCATCCGGGCCATAATGGGGCCCCAGGCCGTGATGGCACAGATGGCACCC | |
| | CTGGTGAGAAGGGTGAGAAAGGAGATCCAGGTCTTATTCGTCCTAAGGGAGACATCGG | |
| | TGAAACCGGAGTACCCGGGGCTGAAGGTCCCCGAGGCTTTCCGGGAATCCAAGGCAGG | |
| | AAAGGAGAACCTGGAGAAGCTGCCTATGTATACCGCTCAGCATTCAGTGTGGGATTCG | |
| | AGACTTACGTTACTATCCCCAACATGCCCATTCGCTTTACCAAGATCTTCTACAATCA | |
| | GCAAAACCACTATGATGGCTCCACTGGTAAATTCCACTGCAACATTCCTGGGCTGTAC | |
| | TACTTTGCCTACCACATCACAGTCTATATGAAGGATOTGAAGGTCAGCCTCTTCAAGA | |
| | AGGACAAGGCTATGCTCTTCACCTATGATCAGTACCAGGAAAATAATGTGGACCAGGC | |
| | CTCCGGCTCTGTGCTCCTGCATCTGGAGGTGGGCGACCAAGTCTGGCTCCAGGTGTAT | |
| | GGGGAAGGAGAGCGTAATGGACTCTATGCTGATAATGACAATGACTCCACCTTCACAG | |
| | GCTTTCTTCTCTACCATGACACCAACTGATCACCACTAACTCAGAGCCTCCTCCAGGC | |
| | CAAACAGCCCCAAAGTCAATTAAAGGCTTTCAGTACGGTTAGGAAGTTGATTATTATT | |
| | TAGTTGGAGGCCTTTAGATATTATTCATTCATTTACTCATTCATTTATTCATTCATTC | |
| | ATCAAGTAACTTTAAAAAAATCATATGCTATGTTCCCAGTCCTGGGGAGCTTCACAAA | |
| | CATGACCAGATAACTGACTAGAAAGAAGTAGTTGACAGTGCTATTTTGTGCCCACTGT | |
| | CTCTCCTGATGCTCATATCAATCCTATAAGGCACAGGGAACAAGCATTCTCCTGTTTT | |
| | TACAGATTGTATCCTGAGGCTGAGAGACTTAAGTGAATGTCTAAGGTCACACAAGTAT | |
| | TAAGTGACAGTGCTAGAAATCAAACCCAGAGCTGTGCACTTTGTTCACTAGACTGTGC | |
| | CCTTTTATAGAGGTACATGTTCTCTTTGGAGTGTTGGTAGGTGTCTGTTTCCCACCTC | |
| | ACCTGAGAGCCATTGAATTTGCCTTCCTCATGAATTAAAACCTCCCCCAAGCAGAGCT | |
| | TCCTCACACAAAGTGGTTCTATGATGAAGTCCTGTCTTGGAAGGACTACTACTCAATG | |
| | GCCCCTGCACTACTCTACTTCCTCTTACCTATGTCCCTTCTCATGCCTTTCCCTCCAA | |
| | CGGGGAAAGCCAACTCCATCTCTAAGTGCTGAACTCATCCCTCTTCCTCAAGGCCACC | |
| | TGGCCACGAGCTTCTCTGATGTGATATCCACTTTTTTTTTTTTTTGAGATGGAGTCTC | |
| | ACTCTGTCACCCAGGCTGGAGTACAGTGACACGACCTCGGCTCACTCCAGCCTCCTTC | |
| | TCCTGGGTCCAAGCAATTATTGTGCCTCAGCCTCCCGAGTAGCTGAGACTTCAGGTGC | |
| | ATTCCACCACACATGGCTAATTTTTGTATTTTTAGTAGAAATGGOGTTTCGTCATGTT | |
| | GGCCAGGCTGGTCTCGAACTCCTGGCCTAGGTGATCCACCCGCCTCGACCTCCCAAAG | |
| | TGCTGGGATTACAGGCATGAGCCACCATGCCCAGTCGATATCTCACTTTTTATTTTGC | |
| | CATGGATGAGAGTCCTGGGTGTGAGGAACACCTCCCACCAGGCTAGAGGCAACTGCCC | |
| | AGGAAGGACTGTGCTTCCGTCACCTCTAAATCCCTTGCAGATCCTTGATAAATGCCTC | |
| | ATGAAGACCAATCTCTTGAATCCCGTATCTACCCAGAATTAACTCCATTCCACTCTCT | |
| | GCATGTAATCAGTTTTATCCACAGAAACATTTTCATTTTAGGAAATCCCTGGTTTTAA | |
| | GTATCAATCCTTGTTCAGCTGGACAATATCAATCTTTTCCACTGAAGTTAGGGATGAC | |
| | TGTGATTTTCAGAACACGTCCAGAATTTTTCATCAAGAAGGTAGCTTGAGCCTGAAAT | |
| | GCAAAACCCATGGAGGAATTCTGAAGCCATTGTCTCCTTGAGTACCAACAGGGTCAGG | |
| | GAAGACTGGGCCTCCTGAATTTATTATTGTTCTTTAAGAATTACAGGTTGAGGTAGTT | |
| | GATGGTGGTAAACATTCTCTCAGCAGACAATAACTCCAGTGATGTTCTTCAAAGATTT | |
| | TAGCAAAAACAGAGTAAATAGCATTCTCTATCAATATATAAATTTAAAAAACTATCTT | |
| | TTTGCTTACAGTTTTAAATCCTGAACAATTCTCTCTTACATGTGTATTGCTAATCATT | |
| | AAGGTATTATTTTTTCCACATATAAAGCTTTGTCTTTTTGTTGTTGTTGTTGTTTTTA | |
| | AGATGGAGTTTCCCTCTGTTGCCAGGCTAGAGTCCAGTGGCATGATCTCGGCTTACTG | |
| | CAACCTTTGCCTCCCACGTTCAACCGATTCTTCTGCCTCAGCCTCCCGAGTAGCTGGG | |
| | ACCACAGGTCCCTACCACCATGCCAGGCTAATTTTTGTATTTTTAGTAAAGACAGGCT | |
| | TTCACCATATTGGCCAGGCTGGTCTCCAACTCCTGACCTTGTGATCTGCCCACCTCCA | |
| | TTTTTGTTGTTATTTTTTGAGAAAGATAGATATGAGGTTTAGAGAGGGATGAAGAGGT | |
| | GAGAGTAAGCCTTGTGTTAGTCAGAACTCTGTGTTGTGAATGTCATTCACAACAGAAA | |
| | ACCCAAAATATTATGCAAACTACTGTAAGCAAGAAAAATAAAGGAAAAATGGAAACAT | |
| | TTATTCCTTTGCATAATAGAAATTACCAGAGTTGTTCTGTCTTTAGATAAGGTTTGAA | |
| | CCAAAGCTCAAAACAATCAAGACCCTTTTCTGTATGTCCTTCTGTTCTGCCTTCCGCA | |
| | GTGTAGGCTTTACCCTCAGGTGCTACACAGTATAGTTCTAGGGTTTCCCTCCCGATAT | |
| | CAAAAAGACTGTGGCCTGCCCAGCTCTCGTATCCCCAAGCCACACCATCTGGCTAAAT | |
| | GGACATCATGTTTTCTGGTGATGCCCAAAGAGGAGAGAGGAAGCTCTCTTTCCCAGAT | |
| | GCCCCAGCAAGTGTAACCTTGCATCTCATTGCTCTGGCTGAGTTGTGTGCCTGTTTCT | |
| | GACCAATCACTGAGTCAGGAGGATGAAATATTCATATTGACTTAATTGCAGCTTAAGT | |
| | TAGGGGTATGTAGAGGTATTTTCCCTAAAGCAAAATTGGGACACTGTTATCAGAAATA | |
| | GGAGAGTGGATGATAGATGCAAAATAATACCTGTCCACAACAAACTCTTAATGCTGTG | |
| | TTTGAGCTTTCATGAGTTTCCCAGAGAGACATAGCTGGAAAATTCCTATTGATTTTCT | |
| | CTAAAATTTCAACAAGTAGCTAAAGTCTGGCTATGCTCACAGTCTCACATCTGGTTGG | |
| | GGTGGGCTCCTTACAGAACACGCTTTCACAGTTACCCTAAACTCTCTGGGGCAGGGTT | |
| | ATTCCTTTGTGGAACCAGAGGCACAGAGAGAGTCAACTGAGGCCAAAAGAGGCCTGAG | |
| | ACAAACTGAGGTCAAGATTTCAGGATTAATGGTCCTCTGATGCTTTGAAGTACAATTG | |
| | TGGATTTGTCCAATTCTCTTTAGTTCTGTCAGCTTTTGCTTCATATATTTTAGCGCTC | |
| | TATTATTAGATATATACATGTTTAGTATTATCTCTTATTGGTGCATTTACTCTCTTAT | |
| | CATTATGTAATGTCCTTCTTTATCTGTGATAATTTTCTGTGTTCTGAAGTCTACTTTG | |
| | TCTAAAAATAACATACGCACTCAACTTCCTTTTCTTTCTTCCTTCCTTTCTTTCTTCC | |
| | TTCCTTTCTTTCTCTCTCTCTCTCTTTCCTTCCTTCCTTCCTCCTTTTCTTTCTCTCT | |
| | CTCTCTCTCTCTCTTTTTTTGACAGACTCTCGTTCTGTGGCCCTGGCTGGAGTTCAGT | |
| | GGTGTGATCTTGGCTCACTGCTACCTCTACCATGAGCAATTCTCCTGCCTCAGCCTCC | |
| | CAAGTAGCTGGAACTACAGGCTCATGCCACTGCGCCCAGCTAATTTTTGTATTTTTCG | |
| | TAGAGACGGGGTTTCACCACATTCGTCAGGTTGGTTTCAAACTCCTGACTTTGTGATC | |
| | CACCCGCCTCGGCCTCCCAAAGTGCTGGGATTACAGGCATGAGCCATCACACCTGGTC | |
| | AACTTTCTTTTGATTAGTGTTTTTGTGGTATATCTTTTTCCATCATGTTACTTTAAAT | |
| | ATATCTATATTATTGTATTTAAAATGTGTTTCTTACAGACTGCATGTAGTTGGGTATA | |
| | ATTTTTATCCAGTCTAAAAATATCTGTCTTTTAATTGGTGTTTAGACAATTTATATTT | |
| | AATAAAATTGTTGAATTTAAG | |
| | ORF Start: ATG at 49 | ORF Stop: TGA at 781 |
| | SEQ ID NO:8 | 244 aa MW at 26413.5 kD |
| NOV2b, | MLLLGAVLLLLALPGHDQETTTQGPGVLLPLPKGACTGWMAGIPGHPGHNGAPGRDGR | |
| CG127779-01 | DGTPGEKGEKGDPGLIGPKGDICETGVPGAEGPRGFPGIQGRKGEPGEGAYVYRSAFS | |
| Protein Sequence | VGLETYVTIPNMPIRFTKIFYNQQNHYDGSTGKFHCNIPGLYYFAYHITVYMKDVKVS | |
| | LFKKDKAMLFTYDQYQENNVDQASGSVLLHLEVGDQVWLQVYGEGERNGLYADNDNDS | |
| | TFTGFLLYHDTN | |
| | SEQ ID NO:9 | 754 bp |
| NOV2c, | CACCGGATCCACCATGCTGTTGCTGGGAGCTGTTCTACTGCTATTAGCTCTGCCCGGT | |
| CG127779-03 | CATGACCAGGAACCACGACTCAAGCGCCCGGAGTCCTGCTTCCCCTGCCCAAGGGGG | |
| DNA Sequence | CCTGCACAGGTTGGATGGCGGGCATCCCAGGGCATCCGGGCCATAATGOGGCCCCAGG | |
| | CCGTGATGGCAGAGATGGCACCCCTGGTGAGAAGGGTGAGAAAGGAGATCCAGGTCTT | |
| | ATTGGTCCTAAGGGAGACATCGGTGAAACCGGAGTACCCGGGGCTGAAGGTCCCCGAG | |
| | GCTTTCCGGGAATCCAAGGCAGCAAAGGAGAACCTGGAGAAGGTGCCTATGTATACCG | |
| | CTCAGCATTCAGTGTGGGATTGOAGACTTACGTTACTATCCCCAACATGCCCATTCGC | |
| | TTTACCAAGATCTTCTACAATCAGCAAAACCACTATGATGGCTCCACTGGTAAATTCC | |
| | ACTGCAACATTCCTGGGCTGTACTACTTTGCCTACCACATCACAGTCTATATGAAGGA | |
| | TGTGAAGGTCAGCCTCTTCAAGAAGGACAAGGCTATGCTCTTCACCTATGATCAGTAC | |
| | CAGGAAAATAATGTGGACCAGGCCTCCGGCTCTGTGCTCCTGCATCTGGAGGTCGGCG | |
| | ACCAAGTCTGGCTCCAGGTGTATGGGGAAGGAGAGCGTAATGGACTCTATGCTGATAA | |
| | TGACAATGACTCCACCTTCACAGGCTTTCTTCTCTACCATGACACCAACCTCGAGGGC | |
| | ORF Start: ATG at 14 | ORF Stop: at 746 |
| | SEQ ID NO:10 | 244 aa MW at 26413.5 kD |
| NOV2c, | MLLLGAVLLLLALPGHDQETTTQGPGVLLPLPKGACTGWMAGIPGHPGHNGAPGRDGR | |
| CG12777903 | DGTPGEKGEKGDPGLIGPKODIGETGVPGAEGPRGFPGIQGRKCEPGEGAYVYRSAFS | |
| Protein Sequence | VGLETYVTIPNNPIRFTKIFYNQQNHYDGSTGKFHCNIPGLYYFAYHITVYMKDVKVS | |
| | TFTGFLLYHDTN | |
| | SEQ ID NO:11 | 778 bp |
| NOV2d, | CACCGGATCCACCATGGGCCACCATCACCACCATCACCTGTTGCTGGGAGCTGTTCTA | |
| CG127779-04 | CTGCTATTAGCTCTGCCCGGTCATGACCAGGAAACCACGACTCAAGGGCCCGGAGTCC | |
| DNA Sequence | TGCTTCCCCTGCCCAAGGGGGCCTGCACAGGTTGGATGGCGGGCATCCCAGGGCATCC | |
| | GCGCCATAATGGGGCCCCAGGCCGTGATGGCAGAGATCGCACCCCTGGTGAGAAGGGT | |
| | GAGAAAGGAGATCCAGGTCTTATTGGTCCTAAGGGAGACATCGGTGAAACCGGAGTAC | |
| | CCGGGGCTGAAGGTCCCCGAGGCTTTCCGGGAATCCAAGGCAGGAAAGGAGAACCTGG | |
| | AGAAGGTGCCTATGTATACCGCTCAGCATTCAGTGTGGGATTGGAGACTTACGTTACT | |
| | ATCCCCAACATGCCCATTCGCTTTACCAAGATCTTCTACAATCAGCAAAACCACTATG | |
| | ATGGCTCCACTGGTAAATTCCACTGCAACATTCCTGGGCTGTACTACTTTGCCTACCA | |
| | CATCACAGTCTATATGAAGGATGTGAACGTCAGCCTCTTCAAGAAGGACAAGGCTATG | |
| | CTCTTCACCTATGATCAGTACCAGGAAAATAATGTGGACCAGGCCTCCGGCTCTGTGC | |
| | TCCTGCATCTGGAGGTGGGCGACCAAGTCTGGCTCCAGGTGTATGGGGAAGGAGAGCG | |
| | TAATGGACTCTATGCTGATAATGACAATGACTCCACCTTCACAGGCTTTCTTCTCTAC | |
| | CATGACACCAACTAACTCGAGGGC | |
| | ORF Start: at 17 | ORF Stop: TAA at 767 |
| | SEQ ID NO:12 | 250 aa MW at 27162.2 kD |
| NOV2d, | GHHHHHHLLLGAVLLLLALPGHDQETTTQGPGVLLPLPKGACTGWMAGIPGHPGHNGA | |
| CG127779-04 | PGRDGRGTPGEKGEKGDPGLIGPKGDIGETGVPGAEGPRGFPGIQGRKGEPGEGAYV | |
| Protein Sequence | YRSAFSVGLETYVTIPNMPIRFTKIFYNQQNHYDGSTGKFHCNIPGLYYFAYHITVYM | |
| | KDVVSLFKKDKAMLFTYDQYQENNVDQASGSVLLHLEVGDQVWLQVYGEGERNGLYA | |
| | DNDNDSTFTGFLLYHDTN | |
| | SEQ ID NO:13 | 742 bp |
| NOV2e, | CACCGGATCCCACCATCACCACCATCACCAGGAACCACGACTCAAGGGCCCGGAAGTC | |
| CG127779-05 | CTGCTTCCCCTGCCCAAGGGGGCCTGCACAGGTTGGATGGCGGCATCCCAGGGCATC | |
| DNA Sequence | CGGGCCATAATGGGGCCCCAGGCCGTGATGGCAGAGATGGCACCCCTGGTGAGAAGGG | |
| | TGAGAAAGGAGATCCACGTCTTATTGGTCCTAAGGGAGACATCGGTGAAACCGGAGTA | |
| | CCCGGGGCTGAAGGTCCCCGAGGCTTTCCGGGAATCCAAGGCAGGAAAGGAGAACCTG | |
| | GAGAAGGTGCCTATGTATACCGCTCAGCATTCAGTGTGGGATTGGAGACTTACGTTAC | |
| | TATCCCCAACATGCCCATTCGCTTTACCAAGATCTTCTACAATCAGCAAAACCACTAT | |
| | GATGGCTCCACTGGTAAATTCCACTGCAACATTCCTGGGCTGTACTACTTTGCCTACC | |
| | ACATCACAGTCTATATGAAGGATGTGAAGGTCAGCCTCTTCAAGAAGGACAAGGCTAT | |
| | GCTCTTCACCTATGATCAGTACCAGGAAAATAATGTGGACCAGGCCTCCGGCTCTGTG | |
| | CTCCTGCATCTGGAGGTGGGCGACCAAGTCTGGCTCCAGGTGTATGGGGAAGGAGAGC | |
| | GTAATGGACTCTATGCTGATAATGACAATGACTCCACCTTCACAGGCTTTCTTCTCTA | |
| | CCATGACACCAACTAACTCGAGGGCAAGGGCGAATTCCAGACACT | |
| | ORF Start: at 11 | ORF Stop: TAA at 710 |
| | SEQ ID NO:14 | 233 aa MW at 25495.2 kD |

| | |
|---|---|
| NOV2e, | HHHHHHQETTTQGPGVLLPLPKGACTGWMAGIPGHPGHNGAPGRDGRDGTPGEKGEKG |
| CG127779-05 | DPCLIGPKGDIGETGVPGAEGPRGFPGIQGRKGEPGEGAYVYRSAFSVGLETYVTIPN |
| Protein Sequence | MPIRFTKIFYNQQNHYDGSTGKFHCNIPGLYYFAYHITVYMKDVKVSLFKKDKANLFT |
| | YDQYQENNVDQASGSVLLHLEVGDQVWLQVYGEGERNGLYADNDNDSTFTGFLLYHDT |
| N | |

Sequence comparison of the above protein sequences yields the following sequence relationships shown in Table 2B.

TABLE 2B
Comparison of NOV2a against NOV2b through NOV2e.

| | NOV2a Residues/ | Identities/Similarities |
|---|---|---|
| Protein Sequence | Match Residues | for the Matched Region |
| NOV2b | 1 . . . 244 | 244/244 (100%) |
| | 1 . . . 244 | 244/244 (100%) |
| NOV2c | 1 . . . 244 | 244/244 (100%) |
| | 1 . . . 244 | 244/244 (100%) |
| NOV2d | 2 . . . 244 | 243/243 (100%) |
| | 8 . . . 250 | 243/243 (100%) |
| NOV2e | 16 . . . 244 | 228/229 (99%) |
| | 5 . . . 233 | 228/229 (99%) |

Further analysis of the NOV2a protein yielded the following properties shown in Table 2C.

TABLE 2C
Protein Sequence Properties NOV2a

| SignalP analysis: | Cleavage site between residues 18 and 19 | |
|---|---|---|
| PSORT II analysis: | PSG: a new signal peptide prediction method | |
| | N-region: length 0; pos.chg 0; neg.chg 0 | |
| | H-region: length 16; peak value 10.35 | |
| | PSG score: 5.95 | |
| | GvH: von Heijne's method for signal seq. recognition | |
| | GvH score (threshold: −2.1): 1.28 | |
| | possible cleavage site: between 14 and 15 | |
| | >>> Seems to have a cleavable signal peptide (1 to 14) | |
| | ALOM: Klein et al's method for TM region allocation | |
| | Init position for calculation: 15 | |
| | Tentative number of TMS(s) for the threshold 0.5: 0 | |
| | number of TMS(s) . . . fixed | |
| | PERIPHERAL Likelihood = 4.61 (at 27) | |
| | ALOM score: 4.61 (number of TMSs: 0) | |
| | MTOP: Prediction of membrane topology (Hertmann et al.) | |
| | Center position for calculation: 7 | |
| | Charge difference: −2.5 C(−1.5) − N(1.0) | |
| | N >= C: N-terminal side will be inside | |
| | MITDISC: discrimination of mitochondrial targeting seq | |
| | R content: 0 | Hyd Moment(75): 2.66 |
| | Hyd Moment(95): 1.21 | G content: 2 |
| | D/E content: 1 | S/T content: 0 |
| | Score: −7.00 | |

Gavel: prediction of cleavage sites for mitochondrial preseq
cleavage site motif not found
NUCDISC: discrimination of nuclear localization signals
pat4: none
pat7: none
bipartite: none
content of basic residues: 7.8%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
prediction: cytoplasmic
Reliability: 94.1
COIL: Lupas's algorithm to detect coiled-coil regions
total: 0 residues

Final Results (k = 9/23):
33.3%: endoplasmic reticulum
22.2%: vacuolar
22.2%: mitochondrial
22.2%: extracellular, including cell wall
>> prediction for CG127779-02 is end (k = 9)

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG80254 | Human APM1 protein - Homo | 1 . . . 244 | 244/244 (100%) | e−148 |
| | sapiens, 244 aa. [WO200132868- | 1 . . . 244 | 244/244 (100%) | |
| | A1, 10-MAY-2001] | | | |
| ABB08223 | Human apm1 protein - Homo | 1 . . . 244 | 244/244 (100%) | e−148 |
| | sapiens, 244 aa. [WO200192330- | 1 . . . 244 | 244/244 (100%) | |
| | A2, 06-DEC-2001] | | | |
| AAB50373 | Human adipocyte complement | 1 . . . 244 | 244/244 (100%) | e−148 |
| | related protein ACRP30 - Homo | 1 . . . 244 | 244/244 (100%) | |
| | sapiens, 244 aa. [WO200073448- | | | |
| | A1, 07-DEC-2000] | | | |
| AAB49598 | Human ACRP30 protein - Homo | 1 . . . 244 | 244/244 (100%) | e−148 |
| | sapiens, 244 aa. [WO200073446- | 1 . . . 244 | 244/244 (100%) | |
| | A2, 07-DEC-2000] | | | |
| AAB65828 | Human adipocyte complement | 1 . . . 244 | 244/244 (100%) | e−148 |
| | mediated protein precursor SEQ ID | 1 . . . 244 | 244/244 (100%) | |
| | NO: 20 - Homo sapiens, 244 aa. | | | |
| | [WO200078808-A1, 28-DEC-2000] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q15848 | Adiponectin precursor (30 kDa | 1 . . . 244 | 244/244 (100%) | e−148 |
| | adipocyte complement-related | 1 . . . 244 | 244/244 (100%) | |
| | protein) (ACRP30) (Adipose most | | | |
| | abundant gene transcript 1) (apM-1) | | | |
| | (Gelatin- binding protein) - Homo | | | |
| | sapiens (Human), 244 aa. | | | |
| Q95JD7 | Adiponectin - Macaca mulatta | 2 . . . 244 | 235/243 (96%) | e−143 |
| | (Rhesus macaque), 243 aa. | 1 . . . 243 | 238/243 (97%) | |
| Q8K3R4 | 30 kDa adipocyte complement- | 1 . . . 244 | 203/244 (83%) | e−124 |
| | related protein - Rattus norvegicus | 1 . . . 244 | 219/244 (89%) | |
| | (Rat), 244 aa. | | | |
| AAH28770 | Adipocyte complement related | 1 . . . 244 | 204/247 (82%) | e−122 |
| | protein of 30 kDa - Mus musculus | 1 . . . 247 | 218/247 (87%) | |
| | (Mouse), 247 aa. | | | |
| Q60994 | Adiponectin precursor (30 kDa | 1 . . . 244 | 203/247 (82%) | e−121 |
| | adipocyte complement-related | 1 . . . 247 | 218/247 (88%) | |
| | protein) (ACRP30) (Adipocyte | | | |
| | specific protein AdipoQ) - Mus | | | |
| | musculus (Mouse), 247 aa. | | | |

PFam analysis predicts that the NOV2a protein contains the domains shown in the Table 2F.

TABLE 2F
Domain Analysis of NOV2a

| | | Identities/ | |
|---|---|---|---|
| | NOV2a | Similarities | Expect |
| Pfam Domain | Match Region | for the Matched Region | Value |
| Collagen | 45 . . . 104 | 29/60 (48%) | 2.3e−10 |
| | | 42/60 (70%) | |
| C1q | 114 . . . 239 | 51/138 (37%) | 1.5e−43 |
| | | 99/138 (72%) | |

### Example 3

The NOV3 clone was analyzed, and the nucleotide and encoded polypeptide sequences are shown in Table 3A.

TABLE 3A
NOV3 Sequence Analysis

| | SEQ ID NO:15 | 387 bp |
|---|---|---|
| NOV3a, | GGATCCGCCTTCTACGCCGGCCTCAAGAACCCCCACGAGGCTTACGAGGTACTCAAGT | |
| CG92035-03 | TTGACGACGTGGTCACCAACCTAGGCAACAACTACGACGCGGCCAGCGGCAAGTTTAC | |
| DNA Sequence | GGCACCAGTATGTGGGCAGACCTCTGCAAGAATGGCCACGTGCGGGCCAGTGCTATTG | |
| | CCCACGACGCGGACCAGAACTACGACTACGCGAGCAACAGCGTGATCCTGCACCTGGA | |
| | CGCCCCTGACGAGGTCTTCATCAAGCTGGATGCACGCAAACCACACCGCGCCAACAGC | |
| | AACAAATACAGCACGTTCTCTGGCTTCATCATCCTCGAG | |
| | ORF Start: at 7 | ORF Stop: at 382 |
| | SEQ ID NO:16 | 125 aa MW at 13576.9 kD |
| NOV3a, | AFYAGLKNPHEGYEVLKFDDVVTNLGNNYDAASGKFTCNIPGTYFFTYHVLMRGGDGT | |
| CG92035-03 | SMWADLCKNGQVRASAIAQDADQNYDYASNSVILHLDACDEVFIKLDCGKAHGGNSNK | |
| Protein Sequence | YSTFSGFII | |
| | SEQ ID NO:17 | 813 bp |
| NOV3b, | GCGCCGGCGGCCGCCGCGGGTGTGCTGATGCTGCTGGTGCTCGTGGTCCTCATCCCCG | |
| CG92035-01 | TGCTGGTGAGCTCGGGCGCCCCGCAAGGCCACTATGAGATGCTCGGCACCTGCCGCAT | |
| DNA Sequence | GGTGTGCGACCCCTACCCCGCGCGCCGCCCCGGCGCCGCCGCGCOGACCGACGGCGGC | |
| | GACGCCCTGACCGAGCAGAGCGGCCCGCCCCCCCCTTCCACGCTCGTGCAGGGCCCCC | |
| | ACGGGAAGCCGGGCCCCACCGCCAAGCCCGGCCCTCCGGGCCCTCCCCGGGACCCAGG | |
| | TCCTCCCGGCCCTGTCGGGCCGCCGGGGGAGAAGGGTCAGCCAGGCAAGCCGGCCCCT | |
| | CCGGGCCTGCCGCCCGCGGGGGOCAGCGGCGCCATCAGCACTGCCACCTACACCACGG | |
| | TGCCCCGCGTCGCCTTCTACGCCCGCCTCAACAACCCCCACCAGGGTTACGAGGTACT | |
| | CAAGTTTGACGACGTGGTCACCAACCTAGGCAACAACTACGACGCGGCCACCGGCAAG | |
| | TTAACGTGCAACATTCCCGGCACCTACTTTTTCACCTACCATGTCCTCATCCGCCGCG | |
| | GCGACGGCACCAGTATGTGGGCAGACCTCTGCAAGAATGGCCAGGTGCGGGCCAGTGC | |
| | TATTGCCCAGGACGCGGACCAGAACTACGACTACGCCAGCAACAGCCTGATCCTGCAC | |
| | CTGGACGCCCGCGACCAGGTCTTCATCAAGCTGGATGGAGGCAAAGCACACCGCCGCA | |
| | ACAGCAACAAATACACCACGTTCTCTGGCTTCATCATCTACTCCGACTGACCTCCCCA | |
| | C | |
| | ORF Start: ATG at 28 | ORF Stop: TGA at 802 |
| | SEQ ID NO:18 | 258 aa MW at 26418.3 kD |
| NOV3b, | MLLVLVVLIPVLVSSGGPEGHYEMLGTCRMVCDPYPARGPGAGARTDGGDALSEQSGA | |
| CG92035-01 | PPPSTLVQGPQGKPCRTGKPCPPGPPGDPGPPGPVCPPCEKGEPGKPGPPGLPGAGGS | |
| Protein Sequence | GAISTATYTTVPRVAFYAGLKNPHEGYEVLKFDDVVTNLGNNYDAASGKLTCNIPGTY | |
| | FFTYHVLMRGGDGTSMWADLCKNGQVRASAIAQDADQNYDYASNSVILHLDAGDEVFI | |
| | KLDGGKAHGGNSNKYSTFSGFIIYSD | |
| | SEQ ID NO:19 | 795 bp |
| NOV3c, | GGTGTGGTGATGCTGCTGGTGCTGGTGGTGCTCATCCCCGTGCTGGTGAGCTCGGGCG | |
| CG92035-02 | GCCCGGAAGGCCACTATGAGATGCTGGGCACCTGCCGCATGGTGTGCGACCCCTACCC | |
| DNA Sequence | CGCGCGGCGCCCCGGCGCCGGCGCGCGGACCGACGGCGGCGACGCCCTGAGCGAGCAG | |
| | AGCGGCGCGCCCCCGCCTTCCACGCTGGTGCAGGGCCCCCAGGGGAAGCCGGGCCGCA | |
| | CCGGCAAGCCCGGCCCTCCGGGGCCTCCCGGGGACCCAGGTCCTCCCGGCCCTGTGGG | |
| | GCCGCCGGGGGAGAAGGGTGAGCCAGGCAAGCCGGGCCCTCCGGGGCTGCCGGGCGCG | |
| | GGGGGCAGCGGCGCCATCAGCACTGCCACCTACACCACGGTGCCGCGCGTGGCCTTCT | |
| | ACCCCGGCCTCAAGAACCCCCACGAGGGTTACGAGGTACTCAAGTTTGACGACGTGGT | |
| | CACCAACCTAGGCAACAACTACGACGCGGCCAGCGGCAAGTTAACGTGCAACATTCCC | |
| | GGCACCTACTTTTTCACCTACCATGTCCTCATGCGCGGCGGCGACGGCACCAGTATGT | |
| | GGGCAGACCTCTGCAAGAATGGCCAGGTGCGGGCCAGTGCTATTGCCCAGGACGCGGA | |
| | CCAGAACTACGACTACGCCAGCAACAGCGTGATCCTGCACCTGGACGCCGGCGACGAG | |
| | GTCTTCATCAAGCTGGATGGAGGCAAAGCACACGGCGGCAACAGCAACAAATACAGCA | |
| | CGTTCTCTGGCTTCATCATCTACTCCGACTGAGCTCCCCAC | |
| | ORF Start: at 28 | ORF Stop: TGA at 784 |
| | SEQ ID NO:20 | 252 aa MW at 25749.4 kD |

| | |
|---|---|
| NOV3c, | VLIPVLVSSGGPEGHYEMLGTCRMVCDPYPARGPGAGARTDGGDALSEQSGAPPPSTL |
| CG92035-02 | VQGPQGKPGRTGKPGPPGPPGDPGPPGPVGPPGEKGEPGKPGPPGLPGACGSGAISTA |
| Protein Sequence | TYTTVPRVAFYAGLKNPHEGYEVLKFDDVVTNLGNNYDAASGKLTCNIPGTYFFTYHV |
| | LMRGGDGTSMWADLCKNGQVRASAIAQDADQNYDYASNSVILHLDAGDEVFIKLDGGK |
| | AHGGNSNKYSTFSGFIIYSD |

Sequence comparison of the above protein sequences yields the following sequence relationships shown in Table 3B.

TABLE 3B
Comparison of NOV3a against NOV3b and NOV3c.

| | NOV3a Residues/ | Identities/Similarities |
|---|---|---|
| Protein Sequence | Match Residues | for the Matched Region |
| NOV3b | 1 . . . 125 | 124/125 (99%) |
| | 131 . . . 255 | 124/125 (99%) |
| NOV3c | 1 . . . 125 | 124/125 (99%) |
| | 125 . . . 249 | 124/125 (99%) |

Further analysis of the NOV3a protein yielded the following properties shown in Table 3C.

TABLE 3C
Protein Sequence Properties NOV3a

| SignalP analysis: | No Known Signal Sequence Predicted | |
|---|---|---|
| PSORT II analysis: | PSG: a new signal peptide prediction method | |
| | N-region: length 11; pos.chg 1; neg.chg 1 | |
| | H-region: length 2; peak value −8.78 | |
| | PSG score: −13.18 | |
| | GvH: von Heijne's method for signal seq. recognition | |
| | GvH score (threshold: −2.1): −8.76 | |
| | possible cleavage site: between 57 and 58 | |
| | >>> Seems to have no N-terminal signal peptide | |
| | ALOM: Klein et al's method for TM region allocation | |
| | Init position for calculation: 1 | |
| | Tentative number of TMS(s) for the threshold 0.5: 0 | |
| | number of TMS(s) . . . fixed | |
| | PERIPHERAL Likelihood = 7.80 (at 86) | |
| | ALOM score: 7.80 (number of TMSs: 0) | |
| | MITDISC: discrimination of mitochondrial targeting seq | |
| | R content: 0 | Hyd Moment(75): 6.67 |
| | Hyd Moment(95): 7.94 | G content: 2 |
| | D/E content: 2 | S/T content: 0 |
| | Score: −7.71 | |

Gavel: prediction of cleavage sites for mitochondrial preseq
cleavage site motif not found
NUCDISC: discrimination of nuclear localization signals
pat4: none
pat7: none
bipartite: none
content of basic residues: 7.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues

Final Results (k = 9/23):
43.5%: cytoplasmic
43.5%: nuclear
13.0%: mitochondrial
>> prediction for CG92035-03 is cyt (k = 23)

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| ABB53290 | Human polypeptide #30 - Homo | 1 . . . 125 | 111/125 (88%) | 1e−63 |
| | sapiens, 255 aa. [WO200181363-A1, | 128 . . . 252 | 118/125 (93%) | |
| | 01-NOV-2001] | | | |
| ABG79646 | Human novel secreted protein | 1 . . . 125 | 111/125 (88%) | 1e−63 |
| | SECP22, Incyte ID No. | 128 . . . 252 | 118/125 (93%) | |
| | 5960119CD1 - Homo sapiens, 255 | | | |
| | aa. [WO200262841-A2, 15-AUG-2002] | | | |
| AAG64212 | Murine HSP47 interacting protein, | 1 . . . 125 | 111/125 (88%) | 1e−63 |
| | #2 - Mus sp, 255 aa. | 128 . . . 252 | 118/125 (93%) | |
| | [JP2001145493-A, 29-MAY-2001] | | | |
| AAU09865 | Novel human secreted protein #6 - | 1 . . . 125 | 106/125 (84%) | 3e−63 |
| | Homo sapiens, 287 aa. | 160 . . . 284 | 119/125 (94%) | |
| | [WO200179454-A1, 25-OCT-2001] | | | |
| AAU76873 | Human CRF-like protein LP231 - | 1 . . . 125 | 106/125 (84%) | 3e−63 |
| | Homo sapiens, 225 aa. | 98 . . . 222 | 119/125 (94%) | |
| | [WO200212475-A2, 14-FEB-2002] | | | |

In a BLAST search of public sequence datbases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O88992 | C1q-related factor precursor - | 1 . . . 125 | 125/125 (100%) | 9e−71 |
| | Mus musculus (Mouse), 258 aa. | 131 . . . 255 | 125/125 (100%) | |
| O75973 | C1q-related factor precursor - | 1 . . . 125 | 125/125 (100%) | 9e−71 |
| | Homo sapiens (Human), 258 aa. | 131 . . . 255 | 125/125 (100%) | |
| Q8R1Z2 | Hypothetical 13.1 kDa protein - | 9 . . . 125 | 117/117 (100%) | 8e−66 |
| | Mus musculus (Mouse), 120 aa | 1 . . . 117 | 117/117 (100%) | |
| | (fragment). | | | |
| Q9ESN4 | Gliacolin precursor (C1q-like | 1 . . . 125 | 111/125 (88%) | 3e−63 |
| | protein) - Mus musculus | 128 . . . 252 | 118/125 (93%) | |
| | (Mouse), 255 aa. | | | |
| AAH40774 | Similar to C1q-like - Mus | 1 . . . 125 | 106/125 (84%) | 9e−63 |
| | musculus (Mouse), 287 aa. | 160 . . . 284 | 119/125 (94%) | |

PFam analysis predicts that the NOV3a protein contains the domains shown in the Table 3F.

TABLE 3F
Domain Analysis of NOV3a

| | | Identities/ | |
|---|---|---|---|
| | NOV3a | Similarities | Expect |
| Pfam Domain | Match Region | for the Matched Region | Value |
| C1q | 1 . . . 125 | 43/140 (31%) | 1.3e−29 |
| | | 92/140 (66%) | |

### Example 4

The NOV4 clone was analyzed, and the nucleotide and encoded polypeptide sequences are shown in Table 4A.

TABLE 4A
NOV4 Sequence Analysis

| | SEQ ID NO:21 | 891 bp |
|---|---|---|
| NOV4a, | ATGGAGTCGATGGCCGTCGCTACCGACGGCGGGGAGAGGCCGGGGGTCCCAGCGGGCT | |
| CG169230-01 | CAGGTCTGTCCGCTTCCCAGCGTCGGGCGGAGCTGCGTCGGAGAAAGCTGCTCATCAA | |
| DNA Sequence | CTCGGAACAGCGCATCAACCGGATCATGGGCTTTCACAGGCCCGGGAGCGGCGCGGAA | |
| | GAAGAAAGTCAAACAAAATCAAAGCAGCAGGACAGTGATAAACTGAACTCCCTCAGCG | |
| | TTCCTTCCGTTTCAAAGCGAGTAGTGCTGGGTGATTCAGTCAGTACAGGAACAACTGA | |
| | CCAGCAGGGTGGTGTGGCCGAGGTAAAGGGGACCCAACTGGGAGACAAATTGGACTCG | |
| | TTCATTAAACCACCTGAGTGCAGTAGTGATGTCAACCTTGAGCTCCGGCAGCGGAACA | |
| | GAGGGCACCTGACAGCGGACTCGGTCCAGAGGGGTTCCCGCCATGCCCTAGAGCAGTA | |
| | CCTTTCCAGATTCGAACAAGCAATGAAGCTAACGAAACAGCTGATTAGTGAAAAACCC | |
| | AGTCAAGAGGATGGAAATACAACAGAAGAATTTCACTCTTTTCGAATATTTAGATTGG | |
| | TGGGATGTCCTCTTCTTGCTCTTCGAGTCAGAGCTTTTGTTTGCAAATACTTGTCCAT | |
| | ATTTGCTCCATTTCTTACTTTACAACTTGCGTACATGGGATTATACAAATATTTTCCC | |
| | AAGAGTCAAAAGAAGATAAAGACAACAGTACTAACAGCTGCACTTCTATTGTCGGGAA | |
| | TTCCTGCCGAAGTGATAAATCGATCAATGGATACCTATAGCAAAATGGGCGAAGTCTT | |
| | CACAGATCTCTGTGTCTACTTTTTCACTTTTATCTTTTCTCATGAACTGCTTGATTAT | |
| | TGCGGCTCTGAAGTACCATGA | |
| | ORF Start: ATG at 1 | ORF Stop: TGA at 889 |
| | SEQ ID NO:23 | 296 aa MW at 32952.2 kD |

| | |
|---|---|
| NOV4a, | MESMAVATDGGERPGVPAGSGLSASQRRAELRRRKLLMNSEQRINRIMGFHRPGSGAE |
| CG169230-01 | EESQTKSKQQDSDKLNSLSVPSVSKRVVLGDSVSTGTTDQQGCVAEVKGTQLGDKLDS |
| Protein Sequence | FIKPPECSSDVNLELRQRNRGDLTADSVQRGSRHGLEQYLSRFEEAMKLRKQLISEKP |
| | SQEDGNTTEEFDSFRIFRLVGCALLALGVRAFVCKYLSIFAPFLTLQLAYMGLYKYFP |
| | KSEKKIKTTVLTAALLLSCIPAEVINRSMDTYSKMGEVFTDLCVYFFTFIFCHELLDY |
| | WGSEVP |

Further analysis of the NOV4a protein yielded the following properties shown in Table 4B.

TABLE 4B
Protein Sequence Properties NOV4a

| SignalP analysis: | No Known Signal Sequence Predicted | |
|---|---|---|
| PSORT II analysis: | PSG: a new signal peptide prediction method | |
| | N-region: length 9; pos.chg 0; neg.chg 2 | |
| | H-region: length 2; peak value 0.00 | |
| | PSG score: −4.40 | |
| | GvH: von Heijne's method for signal seq. recognition | |
| | GvH score (threshold: −2.1): −10.15 | |
| | possible cleavage site: between 56 and 57 | |
| | >>> Seems to have no N-terminal signal peptide | |
| | ALOM: Klein et al's method for TM region allocation | |
| | Init position for calculation: 1 | |
| | Tentative number of TMS(s) for the threshold 0.5: 3 | |
| | Number of TMS(s) for threshold 0.5: 1 | |
| | INTEGRAL Likelihood = −2.60 Transmembrane 190-206 | |
| | PERIPHERAL Likelihood = 0.69 (at 268) | |
| | ALOM score: −2.60 (number of TMSs: 1) | |
| | MTOP: Prediction of membrane topology (Hartmann et al.) | |
| | Center position for calculation: 197 | |
| | Charge difference: 4.0 C(2.0) − N(−2.0) | |
| | C > N: C-terminal side will be inside | |
| | >>> membrane topology: type 1b (cytoplasmic tail 190 to 296) | |
| | MITDISC: discrimination of mitochondrial targeting seq | |
| | R content: 0 | Hyd Moment(75): 4.62 |
| | Hyd Moment(95): 5.86 | G content: 0 |
| | D/E content: 2 | S/T content: 2 |
| | Score: −6.68 | |

Gavel: prediction of cleavage sites for mitochondrial preseq
cleavage site motif not found
NUCDISC: discrimination of nuclear localization signals
pat4: RRRK (5) at 32
pat7: none
bipartite: none
content of basic residues: 13.2%
NLS Score: −0.16
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 198
LL at 247
LL at 248
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues

```
Final Results (k = 9/23):
43.5%: nuclear
21.7%: cytoplasmic
13.0%: mitochondrial
  8.7%: vesicles of secretory system
  4.3%: vacuolar
  4.3%: endoplasmic reticulum
  4.3%: peroxisomal
>> prediction for CG169230-01 is nuc (k = 23)
```

A search of the NOV4a protein against the Geneseq database, a proprietary database that contains sequences published in patents and patent publication, yielded several homologous proteins shown in Table 4C.

TABLE 4C
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR87038 | Human calcium signal-modulating | 1 . . . 296 | 296/296 (100%) | e−168 |
| | cyclophilin ligand - Homo sapiens, | 1 . . . 296 | 296/296 (100%) | |
| | 296 aa. [WO9535501-A1, 28-DEC-1995] | | | |
| ABG96439 | Human ovarian cancer marker | 1 . . . 231 | 225/231 (97%) | e−124 |
| | OV76 - Homo sapiens, 234 aa. | 1 . . . 231 | 227/231 (97%) | |
| | [WO200271928-A2, 19-SEP-2002] | | | |
| ABG04649 | Novel human diagnostic protein | 1 . . . 211 | 211/242 (87%) | e−111 |
| | #4640 - Homo sapiens, 601 aa. | 357 . . . 598 | 211/242 (87%) | |
| | [WO200175067-A2, 11-OCT-2001] | | | |
| ABG44094 | Human peptide encoded by | 59 . . . 211 | 153/153 (100%) | 7e−82 |
| | genome-derived single exon probe | 1 . . . 153 | 153/153 (100%) | |
| | SEQ ID 33759 - Homo sapiens, | | | |
| | 153 aa. [WO200186003-A2, 15-NOV-2001] | | | |
| AAM34297 | Peptide #8334 encoded by probe | 59 . . . 211 | 153/153 (100%) | 7e−82 |
| | for measuring placental gene | 1 . . . 153 | 153/153 (100%) | |
| | expression - Homo sapiens, 153 aa. | | | |
| | [WO200157272-A2, 09-AUG-2001] | | | |

In a BLAST search of public sequence datbases, the NOV4a protein was found to have homology to the proteins shown in the BLASTP data in Table 4D.

TABLE 4D
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P49069 | Calcium-signal modulating | 1 . . . 296 | 296/296 (100%) | e−168 |
| | cyclophilin ligand (CAML) - | 1 . . . 296 | 296/296 (100%) | |
| | Homo sapiens (Human), 296 aa. | | | |
| Q99JU5 | Calcium modulating ligand - Mus | 1 . . . 296 | 263/296 (88%) | e−147 |
| | musculus (Mouse), 294 aa. | 1 . . . 294 | 270/296 (90%) | |
| P49070 | Calcium-signal modulating | 1 . . . 296 | 262/296 (88%) | e−146 |
| | cyclophilin ligand (CAML) - Mus | 1 . . . 294 | 269/296 (90%) | |
| | musculus (Mouse), 294 aa. | | | |
| Q9ERK1 | Calcium-modulating cyclophilin | 1 . . . 296 | 255/297 (85%) | e−139 |
| | ligand - Rattus norvegicus (Rat), | 1 . . . 294 | 264/297 (88%) | |
| | 294 aa. | | | |
| Q90700 | Calcium-signal modulating | 10 . . . 296 | 211/290 (72%) | e−113 |
| | cyclophilin ligand (CAML) - | 8 . . . 290 | 240/290 (82%) | |
| | Gallus gallus (Chicken), 290 aa. | | | |

PFam analysis predicts that the NOV4a protein contains the domains shown in the Table 4E.

TABLE 4E
Domain Analysis of NOV4a

| | | | |
|---|---|---|---|
| | | Identities/ | |
| | NOV4a | Similarities | Expect |
| Pfam Domain | Match Region | for the Matched Region | Value |

No Significant Matches Found

### Example 5

The NOV5 clone was analyzed, and the nucleotide and encoded polypeptide sequences are shown in Table 5A.

TABLE 5A
NOV5 Sequence Analysis

| | SEQ ID NO:23 | 399 bp |
|---|---|---|
| NOV5a, | ATGCATGTCACCCGCTTACTCCTGGCCACCCTGCTCGTCTTCCTCTGCTTCTTCACTG | |
| CG169422-01 | CCAACAGCCACCTGCCACCTGAGGAGAAGCTCCGAGATGACAGGAGCCTGAGAAGCAA | |
| DNA Sequence | CTCCTCTGTGAACCTACTGGATGTCCCTTCTGTCTCTATTGTGGCGCTGAACAAGAAA | |
| | TCCAAACCGATCGGCAGAAAAGCAGCAGAAAGAAAAGATCTTCTAAGAAGGAGGCTT | |
| | CGATGAAGAAAGTGGTGCGGCCCCGGACCCCCCTATCTGCGCCCTGCGTGGCCACCCG | |
| | CAACAGCTGCAAGCCGCCGGCACCCGCCTGCTGCGACCCGTGCGCCTCCTGCCAGTGC | |
| | CGCTTCTTCCGCAGCGCCTGCTCCTGCCGCGTGCTCAGCCTCAACTGCTGA | |
| | ORF Start: ATG at 1 | ORF Stop: TGA at 397 |
| | SEQ ID NO:24 | 132 aa MW at 14483.8 kD |

| | |
|---|---|
| NOV5a, | MDVTRLLLATLLVFLCFFTANSRLPPEEKLRDDRSLRSNSSVNLLDVPSVSIVALNKK |
| CG169422-01 | SKPIGRKAAEKKRSSKKEASMKKVVRPRTPLSAPCVATRNSCKPPAPACCDPCASCQC |
| Protein Sequence | RFFRSACSCRVLSLNC |

Further analysis of the NOV5a protein yielded the following properties shown in Table 5B.

TABLE 5B
Protein Sequence Properties NOV5a

| SignalP analysis: | Cleavage site between residues 23 and 24 | |
|---|---|---|
| PSORT II analysis: | PSG: a new signal peptide prediction method | |
| | N-region: length 5; pos.chg 1; neg.chg 1 | |
| | H-region: length 21; peak value 12.61 | |
| | PSG score: 8.21 | |
| | GvH: von Heijne's method for signal seq. recognition | |
| | GvH score (threshold: −2.1): −2.27 | |
| | possible cleavage site: between 22 and 23 | |
| | >>> Seems to have no N-terminal signal peptide | |
| | ALOM: Klein et et al's method for TM region allocation | |
| | Init position for calculation: 1 | |
| | Tentative number of TMS(s) for the threshold 0.5: 1 | |
| | Number of TMS(s) for threshold 0.5: 1 | |
| | INTEGRAL Likelihood = −5.52 Transmembrane 3-19 | |
| | PERIPHERAL Likelihood = 4.08 (at 39) | |
| | ALOM score: −5.52 (number of TMSs: 1) | |
| | MTOP: Prediction of membrane topology (Hartmann et al.) | |
| | Center position for calculation: 10 | |
| | Charge difference: −0.5 C(0.5) −N(1.0) | |
| | N >= C: N-terminal side will be inside | |
| | >>> membrane topology: type 2 (cytoplasmic tail 1 to 3) | |
| | MITDISC: discrimination of mitochondrial targeting seq | |
| | R content: 1 | Hyd Moment (75): 5.48 |
| | Hyd Moment (95): 10.40 | G content: 0 |
| | D/E content: 2 | S/T content: 4 |
| | Score: −4.51 | |

Gavel: prediction of cleavage sites for mitochondrial preseq
R-2 motif at 15 TRL|LL
NUCDISC: discrimination of nuclear localization signals
pat4: none
pat7: none
bipartite: KKSKPIGRKAAEKKRSS at 57
content of basic residues: 18.2%
NLS Score: 0.02
KDEL: ER retention motif in the C-terminus: none
ER Membrane Retention Signals:
XXRR-like motif in the N-terminus: DVTR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues

```
Final Results (k = 9/23):
39.1%: nuclear
30.4%: mitochondrial
13.0%: cytoplasmic
  4.3%: extracellular, including cell wall
  4.3%: Golgi
  4.3%: plasma membrane
  4.3%: peroxisomal
>> prediction for CG169422-01 is nuc (k = 23)
```

A search of the NOV5a protein against the Geneseq database, a proprietary database that contains sequences published in patents and patent publication, yielded several homologous proteins shown in Table 5C.

TABLE 5C
Geneseq Results for NOV5a

| | | NOV5a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU10690 | Human agouti polypeptide - | 1 . . . 132 | 131/132 (99%) | 4e−72 |
| | Homo sapiens, 132 aa. | 1 . . . 132 | 131/132 (99%) | |
| | [US6310034-B1, 30-OCT-2001] | | | |
| AAW10102 | Human agouti signalling protein - | 1 . . . 132 | 120/132 (90%) | 8e−64 |
| | Homo sapiens, 130 aa. | 1 . . . 130 | 123/132 (92%) | |
| | [WO9700892-A2, 09-JAN-1997] | | | |
| AAU10689 | Murine agouti polypeptide - Mus | 1 . . . 132 | 105/133 (78%) | 7e−53 |
| | musculus, 131 aa. [US6310034- | 1 . . . 131 | 113/133 (84%) | |
| | B1, 30-OCT-2001] | | | |
| AAB13341 | Mouse agouti gene product - Mus | 1 . . . 132 | 105/133 (78%) | 7e−53 |
| | sp, 131 aa. [US6080550-A, 27-JUN-2000] | 1 . . . 131 | 113/133 (84%) | |
| AAW67568 | Mouse agouti protein - Mus sp, | 1 . . . 132 | 105/133 (78%) | 7e−53 |
| | 131 aa. [US5843652-A, 01-DEC-1998] | 1 . . . 131 | 113/133 (84%) | |

In a BLAST search of public sequence datbases, the NOV5a protein was found to have homology to the proteins shown in the BLASTP data in Table 5D.

TABLE 5D
Public BLASTP Results for NOV5a

| | | NOV5a | Identities/ | |
|---|---|---|---|---|
| Protein | | Residues/ | Similarities for | |
| Accession | | Match | the Matched | |
| Number | Protein/Organism/Length | Residues | Portion | Expect Value |
| P42127 | Agouti switch protein precursor | 1 . . . 132 | 131/132 (99%) | 1e−71 |
| | (Agouti signaling protein) - Homo | 1 . . . 132 | 131/132 (99%) | |
| | sapiens (Human), 132 aa. | | | |
| Q9MYU1 | Agouti signalling protein - Sus | 1 . . . 132 | 107/132 (81%) | 4e−55 |
| | scrofa (Pig), 131 aa. | 1 . . . 131 | 115/132 (87%) | |
| P79407 | Agouti switch protein precursor | 1 . . . 132 | 107/132 (81%) | 3e−54 |
| | (Agouti signaling protein) - | 1 . . . 131 | 113/132 (85%) | |
| | Vulpes vulpes (Red fox), 131 aa. | | | |
| Q95MP2 | Agouti-signaling protein - Equus | 1 . . . 132 | 105/133 (78%) | 1e−53 |
| | caballus (Horse), 133 aa. | 1 . . . 133 | 112/133 (83%) | |
| Q03288 | Agouti switch protein precursor | 1 . . . 132 | 105/133 (78%) | 2e−52 |
| | (Agouti signaling protein) - Mus | 1 . . . 131 | 113/133 (84%) | |
| | musculus (Mouse), 131 aa. | | | |

PFam analysis predicts that the NOV5a protein contains the domains shown in the Table 5E.

TABLE 5E
Domain Analysis of NOV5a

| | | Identities/ | |
|---|---|---|---|
| | NOV5a | Similarities | Expect |
| Pfam Domain | Match Region | for the Matched Region | Value |
| agouti | 6 . . . 128 | 65/147 (44%) | 1.1e−69 |
| | | 122/147 (83%) | |

### Example 6

The NOV6 clone was analyzed, and the nucleotide and encoded polypeptide sequences are shown in Table 6A.

TABLE 6A
NOV6 Sequence Analysis

| | SEQ ID NO:25 | 1399 bp |
|---|---|---|
| NOV6a, | ATGCTGACCGCAGCGGTGCTGAGCTGTGCCCTGCTGCTCGCACTGCCTGCCACGCGAG | |
| CG169440-01 | GAGCCCAGATGGGCTTGGCCCCCATGGAGGGCATCAGAAGGCCTGACCAGGCCCTGCT | |
| DNA Sequence | CCCAGAGCTCCCAGGCCTGGGCCTGCGGGCCCCACTGAAGAAGACAACTGCAGAACAG | |
| | GCAGAAGAGCATCTGTTGCAGGAGGCTCAGGCCTTGGCAGAGGTACTAGACCTGCAGG | |
| | ACCGCGAGCCCCGCTCCTCACGTCGCTGCCTAAGGCTGCATGAGTCCTGCCTGGGACA | |
| | CCAGGTGCCTTGCTGTGACCCATGTGCCACGTGCTACTGCCGCTTCTTCAATGCCTTC | |
| | TGCTACTGCCGCAAGCTGGGTACTCCCATGAATCCCTGCAGCCGCACCTAG | |
| | ORF Start: ATG at 1 | ORF Stop: TAG at 397 |
| | SEQ ID NO:26 | 132 aa MW at 14439.6 kD |

| | |
|---|---|
| NOV6a, | MLTAAVLSCALLLALPATRGAQMGLAPMEGIRRPDQALLPELPGLGLRAPLKKTTAEQ |
| CG169440-01 | AEEDLLQEAQALAEVLDLQDREPRSSRRCVRLHESCLGQQVPCCDPCATCYCRFFNAF |
| Protein Sequence | CYCRKLGTAMNPCSRT |

Further analysis of the NOV6a protein yielded the following properties shown in Table 6B.

TABLE 6B
Protein Sequence Properties NOV6a

| SignalP analysis: | Cleavage site between residues 21 and 22 | |
|---|---|---|
| PSORT II analysis: | PSG: a new signal peptide prediction method | |
| | N-region: length 0; pos.chg 0; neg.chg 0 | |
| | H-region: length 18; peak value 9.41 | |
| | PSG score: 5.01 | |
| | GvH: von Heijne's method for signal seq. recognition | |
| | GvH score (threshold: −2.1): 3.29 | |
| | possible cleavage site: between 20 and 21 | |
| | >>> Seems to have a cleavable signal peptide (1 to 20) | |
| | ALOM: Klein at al's method for TM region allocation | |
| | Init position for calculation: 21 | |
| | Tentative number of TMS(s) for the threshold 0.5: 0 | |
| | number of TMS(s) . . . fixed | |
| | PERIPHERAL Likelihood = 7.96 (at 101) | |
| | ALOM score: 7.96 (number of TMSs: 0) | |
| | MTOP: Prediction of membrane topology (Hartmann et al.) | |
| | Center position for calculation: 10 | |
| | Charge difference: 1.0 C(2.0) − N(1.0) | |
| | C > N: C-terminal side will be inside | |
| | >>>Caution: Inconsistent mtop result with signal peptide | |
| | MITDISC: discrimination of mitochondrial targeting seq | |
| | R content: 1 | Hyd Moment(75): 3.37 |
| | Hyd Moment(95): 1.14 | G content: 2 |
| | D/E content: 1 | S/T content: 3 |
| | Score: −5.09 | |

Gavel: prediction of cleavage sites for mitochondrial preseq
R-2 motif at 29 TRG|AQ
NUCDISC: discrimination of nuclear localization signals
pat4: none
pat7: none
bipartite: none
content of basic residues: 11.4%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 89
COIL: Lupas's algorithm to detect coiled-coil regions
51 K 0.70
52 K 0.72
53 T 0.72
54 T 0.72
55 A 0.72
56 E 0.72
57 Q 0.72
58 A 0.72
59 E 0.72
60 E 0.72
61 D 0.72
62 L 0.72
63 L 0.72
64 Q 0.72
65 E 0.72
66 A 0.72
67 Q 0.72
68 A 0.72
69 L 0.72
70 A 0.72
71 E 0.72
72 V 0.72
73 L 0.72
74 D 0.72
75 L 0.72
76 Q 0.72
77 D 0.72
78 R 0.72
79 E 0.72
total: 29 residues

Final Results (k = 9/23):
33.3%: extracellular, including cell wall
33.3%: mitochondrial
33.3%: nuclear
>> prediction for CG169440-01 is exc (k = 9)

A search of the NOV6a protein against the Geneseq database, a proprietary database that contains sequences published in patents and patent publication, yielded several homologous proteins shown in Table 6C.

TABLE 6C
Geneseq Results for NOV6a

| | | NOV6a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG71868 | Melanocortin receptor ligand, | 1 . . . 132 | 132/132 (100%) | 3e−74 |
| | agouti-related protein (AGRP) - | 1 . . . 132 | 132/132 (100%) | |
| | Homo sapiens, 132 aa. | | | |
| | [US6451543-B1, 17-SEP-2002] | | | |
| AAY83186 | Melanocortin receptor MC4 | 1 . . . 132 | 132/132 (100%) | 3e−74 |
| | receptor ligand AGRP - Synthetic, | 1 . . . 132 | 132/132 (100%) | |
| | 132 aa. [WO200012536-A2, 09-MAR-2000] | | | |
| AAW26777 | Human agouti-regulated protein | 1 . . . 132 | 132/132 (100%) | 3e−74 |
| | ART - Homo sapiens, 132 aa. | 1 . . . 132 | 132/132 (100%) | |
| | [WO9743412-A1, 20-NOV-1997] | | | |
| AAU74941 | Human agouti related protein | 1 . . . 132 | 131/132 (99%) | 7e−74 |
| | (AGRP) sequence - Homo sapiens, | 1 . . . 132 | 132/132 (99%) | |
| | 132 aa. [WO200185930-A2, 15-NOV-2001] | | | |
| AAB75125 | Human agouti related protein | 1 . . . 132 | 131/132 (99%) | 7e−74 |
| | (AGRP) SEQ ID NO:1 - Homo | 1 . . . 132 | 132/132 (99%) | |
| | sapiens, 132 aa. [WO200130808-A1, 03-MAY-2001] | | | |

In a BLAST search of public sequence datbases, the NOV6a protein was found to have homology to the proteins shown in the BLASTP data in Table 6D.

TABLE 6D
Public BLASTP Results for NOV6a

| | | NOV6a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O00253 | Agouti-related protein precursor - | 1 . . . 132 | 132/132 (100%) | 9e−74 |
| | Homo sapiens (Human), 132 | 1 . . . 132 | 132/132 (100%) | |
| | aa. | | | |
| P56473 | Agouti-related protein precursor - | 1 . . . 132 | 106/132 (80%) | 7e−57 |
| | Mus musculus (Mouse), 131 | 1 . . . 131 | 115/132 (86%) | |
| | aa. | | | |
| Q9TU18 | Agouti-related protein - Sus | 1 . . . 132 | 104/134 (77%) | 5e−55 |
| | scrofa (Pig), 134 aa. | 1 . . . 134 | 114/134 (84%) | |
| P56413 | Agouti-related protein precursor - | 1 . . . 132 | 101/134 (75%) | 8e−54 |
| | Bos taurus (Bovine), 134 aa. | 1 . . . 134 | 112/134 (83%) | |
| Q9GLM5 | Agouti-related protein - Sus | 14 . . . 132 | 94/121 (77%) | 2e−49 |
| | scrofa (Pig), 121 aa (fragment). | 1 . . . 121 | 103/121 (84%) | |

PFam analysis predicts that the NOV6a protein contains the domains shown in the Table 6E.

TABLE 6E
Domain Analysis of NOV6a

| | | Identities/ | |
|---|---|---|---|
| | NOV6a | Similarities | Expect |
| Pfam Domain | Match Region | for the Matched Region | Value |
| agouti | 1 . . . 122 | 82/147 (56%) | 3.7e−76 |
| | | 122/147 (83%) | |

### Example B

Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof. The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), Al_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S nibosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min. then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1. 1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_Screening_Panel_v1.4, v1.5, v1.6 and 1.7

The plates for Panels 1.4, 1.5, 1.6 and 1.7 include 2 control wells (genomic DNA control and chemistry control) and 88 to 94 wells containing cDNA from various samples. The samples in Panels 1.4, 1.5, 1.6 and 1.7 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, 1.6 and 1.7 are widely available through the American Type Culture Collection (ATCC), a repository for cultured cell lines, and were cultured using the conditions recommended by the ATCC. The normal tissues found on Panels 1.4, 1.5, 1.6 and 1.7 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1. 1, 1.2, and 1.3D.

Panels 2D, 2.2, 2.3 and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

ARDAIS Prostate v 1.0

The plates for ARDAIS prostate 1.0 generally include 2 control wells and 68 test samples composed of RNA isolated from human tissue procured by surgeons working in close cooperation with Ardais Corporation. The tissues are derived from human prostate malignancies and in cases where indicated malignant samples have “matched margins” obtained from noncancerous prostate tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). RNA from unmatched malignant and non-malignant prostate samples were also obtained from Ardais. Additional information from Ardais provides a gross histopathological assessment of tumor differentiation grade and stage. Moreover, most samples include the original surgical pathology report that provides information regarding the clinical state of the patient.

Panel 3D, 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 10 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS)(BA)(MF)=Individual patients

Adj=Adjacent tissue

Match control=adjacent tissues

-M=Male

-F=Female

COPD=Chronic obstructive pulmonary disease

AI.05 Chondrosarcoma

The AI.05 chondrosarcoma plates are comprised of SW1353 cells that had been subjected to serum starvation and treatment with cytokines that are known to induce MMP (1, 3 and 13) synthesis (eg. IL1beta). These treatments include: IL-1beta (10 ng/ml), IL-1beta+TNF-alpha (50 ng/ml), IL-1beta+Oncostatin (50 ng/ml) and PMA (100 ng/ml). The SW1353 cells were obtained from the ATCC (American Type Culture Collection) and were all cultured under standard recommended conditions. The SW 1353 cells were plated at 3×10⁵ cells/ml (in DMEM medium-10% FBS) in 6-well plates. The treatment was done in triplicate, for 6 and 18 h. The supernatants were collected for analysis of MMP 1, 3 and 13 production and for RNA extraction. RNA was prepared from these samples using the standard procedures.

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (less than 1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adiocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Human Metabolic RTQ-PCR Panel

The plates for the Human Metabolic RTQ-PCR Panel include two control wells (genomic DNA control and chemistry control) and 211 cDNAs isolated from human tissues and cell lines with an emphasis on metabolic diseases. This panel is useful for establishing the tissue and cellular expression profiles for genes believed to play a role in the etiology and pathogenesis of obesity and/or diabetes and to confirm differential expression of such genes derived from other methods. Metabolic tissues were obtained from patients enrolled in the CuraGen Gestational Diabetes study and from autopsy tissues from Type II diabetics and age, sex and race-matched control patients. One or more of the following were used to characterize the patients: body mass index [BMI=wt (kg)/ht(m²)], serum glucose, HgbA1c. Cell lines used in this panel are widely available through the American Type Culture Collection (ATCC), a repository for cultured cell lines. RNA from human Pancreatic Islets was also obtained.

In the Gestational Diabetes study, subjects are young (18-40 years), otherwise healthy women with and without gestational diabetes undergoing routine (elective) Caesarian section. After delivery of the infant, when the surgical incisions were being repaired/closed, the obstetrician removed a small sample (less than 1 cc) of the exposed metabolic tissues during the closure of each surgical level. The biopsy material was rinsed in sterile saline, blotted, and then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus), and subcutaneous adipose. Patient descriptions are as follows:

Patient 7—Non-diabetic Caucasian and obese

Patient 8—Non-diabetic Caucasian and obese

Patient 12—Diabetic Caucasian with unknown BMI and on insulin

Patient 13—Diabetic Caucasian, overweight, not on insulin

Patient 15—Diabetic Caucasian, obese, not on insulin

Patient 17—Diabetic Caucasian, normal weight, not on insulin

Patient 18—Diabetic Hispanic, obese, not on insulin

Patient 19—Non-diabetic Caucasian and normal weight

Patient 20—Diabetic Caucasian, overweight, and on insulin

Patient 21—Non-diabetic Caucasian and overweight

Patient 22—Diabetic Caucasian, normal weight, on insulin

Patient 23—Non-diabetic Caucasian and overweight

Patient 25—Diabetic Caucasian, normal weight, not on insulin

Patient 26—Diabetic Caucasian, obese, on insulin

Patient 27—Diabetic Caucasian, obese, on insulin

Total RNA was isolated from metabolic tissues of 12 Type II diabetic patients and 12 matched control patients included hypothalamus, liver, pancreas, small intestine, psoas muscle, diaphragm muscle, visceral adipose, and subcutaneous adipose. The diabetics and non-diabetics were matched for age, sex, ethnicity, and BMI where possible.

The panel also contains pancreatic islets from a 22 year old male patient (with a BMI of 35) obtained from the Diabetes Research Institute at the University of Miami School of Medicine. Islet tissue was processed to total RNA at CuraGen.

Cell lines used in this panel are widely available through the American Type Culture Collection (ATCC), a repository for cultured cell lines, and were cultured at an outside facility. The RNA was extracted at CuraGen according to CuraGen protocols. All samples were then processed at CuraGen to produce single stranded cDNA.

In the labels used to identify tissues in the Human Metabolic panel, the following abbreviations are used:

PI=placenta

Go=greater omentum

Sk=skeletal muscle

Ut=uterus

CC=Caucasian

HI=Hispanic

AA=African American

AS=Asian

Diab=Type II diabetic

Norm=Non-diabetic

Overwt=Overweight; med BMI

Obese=Hi BMI

Low BM=20-25

Med BM=26-30

Hi BMI=Greater than 30

M=Male

#=Patient identifier

Vis.=Visceral

SubQ=Subcutaneous

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V 1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

Panel CNS_Neurodegeneration_V2.0

The plates for Panel CNS_Neurodegeneration_V2.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains sixteen brains from Alzheimer's disease (AD) patients, and twenty-nine brains from “Normal controls” who showed no evidence of dementia prior to death. The twenty-nine normal control brains are divided into two categories: Fourteen controls with no dementia and no Alzheimer's like pathology (Controls) and fifteen controls with no dementia but evidence of severe Alzheimer's like pathology, (specifically senile plaque load rated as level 3 on a scale of 0-3; 0=no evidence of plaques, 3=severe AD senile plaque load). Tissue from the temporal cotex (Broddmann Area 21) was selected for all samples from the Harvard Brain Tissue Resource Center; from the two sample from the Human Brain and Spinal Fluid Resource Center (samples 1 and 2) tissue from the inferior and superior temporal cortex was used; each sample on the panel represents a pool of inferior and superior temporal cortex from an individual patient. The temporal cortex was chosen as it shows a loss of neurons in the intermediate stages of the disease. Selection of a region which is affected in the early stages of Alzheimer's disease (e.g., hippocampus or entorhinal cortex) could potentially result in the examination of gene expression after vulnerable neurons are lost, and missing genes involved in the actual neurodegeneration process.

In the labels employed to identify tissues in the CNS_Neurodegeneration_V2.0 panel, the following abbreviations are used:

AD=Alzheimer's disease brain; patient was demented and showed AD-like pathology upon autopsy

Control=Control brains; patient not demented, showing no neuropathology

AH3=Control brains; pateint not demented but showing sever AD-like pathology

Inf & Sup Temp Ctx Pool=Pool of inferior and superior temporal cortex for a given individual

A. CG103362-02: Complement C3 Precursor.

Expression of gene CG103362-02 was assessed using the primer-probe set Ag6455, described in Table AA.

TABLE AA
Probe Name Ag6455

| | | | Start | SEQ ID |
|---|---|---|---|---|
| Primers | Sequences | Length | Position | No |
| Forward | 5′-tgggcaacggggc-3′ | 13 | 559 | 27 |
| Probe | TET-5′-tacacccagcagctggccttcag-3′-TAMRA | 23 | 594 | 28 |
| Reverse | 5′-cacgaaggccgcaaag-3′ | 16 | 632 | 29 |

CNS_neurodegeneration_v1.0 Summary: Ag6455 Expression of this gene is low/undetectable (CTs>35) across all of the samples on this panel.

General screening_panel_v1.6 Summary: Ag6455 Expression of this gene is low/undetectable (CTs>35) across all of the samples on this panel.

Panel 4.1D Summary: Ag6455 Expression of this gene is low/undetectable (CTs>35) across all of the samples on this panel.

B. CG127779-02: Adiponectin Precursor.

Expression of gene CG127779-02 was assessed using the primer-probe set Ag7906, described in Table BA. Results of the RTQ-PCR runs are shown in Tables BB and BC.

TABLE BA
Probe Name Ag7906

| | | | Start | SEQ ID |
|---|---|---|---|---|
| Primers | Sequences | Length | Position | No |
| Forward | 5′-gtatggggaaggagagcgta-3′ | 20 | 656 | 30 |
| Probe | TET-5′-acaatgactccaccttcacaggcttt-3′-TAMRA | 26 | 697 | 31 |
| Reverse | 5′-agtggtgatcagttggtgtca-3′ | 21 | 734 | 32 |

TABLE BB
General_screening_panel_v1.7
Column A - Rel. Exp.(%) Ag7906, Run 317617341

| Tissue Name | A | Tissue Name | A |
|---|---|---|---|
| Adipose | 100.0 | Gastric ca. (liver met.) NCI-N87 | 0.0 |
| HUVEC | 0.0 | Stomach | 0.0 |
| Melanoma* Hs688(A).T | 0.0 | Colon ca. SW-948 | 0.0 |
| Melanoma* Hs688(B).T | 0.0 | Colon ca. SW480 | 0.0 |
| Melanoma (met) | 0.0 | Colon ca. (SW480 met) SW620 | 0.0 |
| SK-MEL-5 | | | |
| Testis | 0.0 | Colon ca. HT29 | 0.0 |
| Prostate ca. (bone | 0.0 | Colon ca. HCT-116 | 0.0 |
| met) PC-3 | | | |
| Prostate ca. DU145 | 0.0 | Colon cancer tissue | 0.1 |
| Prostate pool | 0.0 | Colon ca. SW1116 | 0.0 |
| Uterus pool | 0.0 | Colon ca. Colo-205 | 0.0 |
| Ovarian ca. OVCAR-3 | 0.0 | Colon ca. SW-48 | 0.0 |
| Ovarian ca. (ascites) | 0.0 | Colon | 0.0 |
| SK-OV-3 | | | |
| Ovarian ca. OVCAR-4 | 0.0 | Small Intestine | 0.0 |
| Ovarian ca. OVCAR-5 | 0.0 | Fetal Heart | 0.0 |
| Ovarian ca. IGROV-1 | 0.0 | Heart | 0.1 |
| Ovarian ca. OVCAR-8 | 0.0 | Lymph Node pool 1 | 0.0 |
| Ovary | 0.4 | Lymph Node pool 2 | 8.7 |
| Breast ca. MCF-7 | 0.0 | Fetal Skeletal Muscle | 0.6 |
| Breast ca. MDA-MB-231 | 0.0 | Skeletal Muscle pool | 0.0 |
| Breast ca. BT-549 | 0.0 | Skeletal Muscle | 0.2 |
| Breast ca. T47D | 0.0 | Spleen | 0.0 |
| Breast pool | 0.0 | Thymus | 0.3 |
| Trachea | 2.1 | CNS cancer (glio/astro) SF-268 | 0.0 |
| Lung | 0.1 | CNS cancer (glio/astro) T98G | 0.0 |
| Fetal Lung | 0.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. NCI-H23 | 0.0 | Brain (Amygdala) | 0.0 |
| Lung ca. NCI-H460 | 0.0 | Brain (Cerebellum) | 0.0 |
| Lung ca. HOP-62 | 0.0 | Brain (Fetal) | 0.0 |
| Lung ca. NCI-H522 | 0.0 | Brain (Hippocampus) | 0.0 |
| Lung ca. DMS-114 | 0.0 | Cerebral Cortex pool | 0.0 |
| Liver | 0.0 | Brain (Substantia nigra) | 0.0 |
| Fetal Liver | 0.0 | Brain (Thalamus) | 0.0 |
| Kidney pool | 0.0 | Brain (Whole) | 0.0 |
| Fetal Kidney | 0.3 | Spinal Cord | 0.0 |
| Renal ca. 786-0 | 0.0 | Adrenal Gland | 1.4 |
| Renal ca. A498 | 0.0 | Pituitary Gland | 0.0 |
| Renal ca. ACHN | 0.0 | Salivary Gland | 0.6 |
| Renal ca. UO-31 | 0.0 | Thyroid | 0.6 |
| Renal ca. TK-10 | 0.0 | Pancreatic ca. PANC-1 | 0.0 |
| Bladder | 0.1 | Pancreas pool | 0.0 |

TABLE BC
Human Metabolic
Column A - Rel. Exp.(%) Ag7906, Run 322940831

| Tissue Name | A | Tissue Name | A |
|---|---|---|---|
| 143503_hypothalamus-AS.M.Diab- | 0.0 | 135765_subQadipose-AS.M.Norm-hi | 1.0 |
| low BMI-20 | | BMI-34 | |
| 143508_hypothalamus-CC.M.Diab- | 0.0 | 135767_subQadipose-CC.M.Norm-hi | 0.2 |
| low BMI-13 | | BMI-29 | |
| 143509_hypothalamus-AA.M.Diab- | 0.0 | 141332_subQadipose-HI.M.Norm-hi | 4.4 |
| low BMI-17 | | BMI-31 | |
| 143505_hypothalamus-HI.M.Diab- | 0.0 | 135768_vis.adipose-AS.M.Norm-low | 0.5 |
| med BMI-23 | | BMI-28 | |
| 143506_hypothalamus-CC.M.Diab- | 0.0 | 137852_vis.adipose-CC.M.Norm-low | 8.8 |
| med BMI-1 | | BMI-39 | |
| 143507_hypothalamus-AS.M.Diab- | 0.0 | 139535_vis.adipose-CC.M.Norm-low | 0.0 |
| hi BMI-9 | | BMI-40 | |
| 143513_hypothalamus-AA.M.Diab- | 0.0 | 139539_vis.adipose-HI.M.Norm-low | 10.5 |
| hi BMI-6 | | BMI-41 | |
| 143515_hypothalamus-CC.M.Diab- | 0.0 | 139530_vis.adipose-HI.M.Norm-med | 0.0 |
| hi BMI-4 | | BMI-35 | |
| 143525_hypothalamus-HI.M.Diab- | 0.0 | 139532_vis.adipose-AA.M.Norm-med | 4.9 |
| hi BMI-21 | | BMI-37 | |
| 134835_diaphragm-AA.M.Diab.- | 2.7 | 139543_vis.adipose-AA.M.Norm-med | 0.0 |
| low BMI-17 | | BMI-47 | |
| 135764_diaphragm-HI.M.Diab.- | 0.1 | 137841_vis.adipose-AA.M.Norm-hi | 5.4 |
| med BMI-23 | | BMI-25 | |
| 137835_diaphragm-CC.M.Diab.- | 0.0 | 137846_vis.adipose-CC.M.Norm-hi BMI-29 | 0.3 |
| med BMI-2 | | | |
| 134828_diaphragm-CC.M.Diab.-hi | 0.0 | 139516_vis.adipose-AS.M.Norm-hi BMI-34 | 0.0 |
| BMI-4 | | | |
| 135761_diaphragm-HI.M.Diab.-hi | 0.6 | 139522_vis.adipose-HI.M.Norm-hi BMI-31 | 27.4 |
| BMI-21 | | | |
| 135772_diaphragm-AS.M.Diab.-hi | 0.7 | 139521_liver-AS.M.Norm-low BMI-28 | 0.0 |
| BMI-9 | | | |
| 137858_diaphragm-AA.M.Diab.-hi | 0.6 | 139534_liver-CC.M.Norm-low BMI-39 | 0.0 |
| BMI-6 | | | |
| 134834_psoas-AA.M.Diab.-low | 0.0 | 139540_liver-HI.M.Norm-low BMI-41 | 0.1 |
| BMI-17 | | | |
| 142740_psoas-AS.M.Diab-low | 0.0 | 141335_liver-CC.M.Norm-med BMI-26 | 0.0 |
| BMI-20 | | | |
| 135763_psoas-HI.M.Diab.-med | 1.1 | 141341_liver-HI.M.Norm-med BMI-35 | 0.0 |
| BMI-23 | | | |
| 137828_psoas-CC.M.Diab.-med | 0.9 | 142741_liver-AA.M.Norm-med BMI-37 | 0.0 |
| BMI-1 | | | |
| 137834_psoas-CC.M.Diab.-med | 0.0 | 137847_liver-HI.M.Norm-hi BMI-31 | 0.0 |
| BMI-2 | | | |
| 137860_psoas-AA.M.Diab.-med | 0.7 | 137849_liver-AS.M.Norm-hi BMI-34 | 0.0 |
| BMI-8 | | | |
| 134827_psoas-CC.M.Diab-hi BMI-4 | 0.0 | 139518_liver-AA.M.Norm-hi BMI-25 | 0.0 |
| 135760_psoas-HI.M.Diab.-hi BMI-21 | 0.0 | 139519_liver-CC.M.Norm-hi BMI-29 | 0.0 |
| 137857_psoas-AA.M.Diab.-hi BMI-6 | 0.0 | 137845_pancreas-AS.M.Norm-low BMI-28 | 0.0 |
| 134832_subQadipose-AA.M.Diab.- | 45.1 | 139533_pancreas-CC.M.Norm-low BMI-39 | 0.0 |
| low BMI-17 | | | |
| 135757_subQadipose-CC.M.Diab.- | 5.7 | 139537_pancreas-CC.M.Norm-low BMI-40 | 0.1 |
| low BMI-13 | | | |
| 139547_subQadipose-HI.M.Diab- | 56.3 | 139541_pancreas-Hi.M.Norm-low BMI-41 | 0.0 |
| low BMI-22 | | | |
| 141338_subQadipose-AS.M.Diab- | 6.9 | 137871_pancreas-CC.M.Norm-med BMI-26 | 0.0 |
| low BMI-20 | | | |
| 135762_subQadipose-HI.M.Diab.- | 0.2 | 139531_pancreas-AA.M.Norm-med | 0.0 |
| med BMI-23 | | BMI-37 | |
| 137862_subQadipose-CC.M.Diab.- | 41.5 | 139545_pancreas-AA.M.Norm-med | 0.0 |
| med BMI-1 | | BMI-47 | |
| 141329_subQadipose-AA.M.Diab- | 15.4 | 142744_pancreas-HI.M.Norm-med BMI-35 | 0.0 |
| med BMI-8 | | | |
| 135771_subQadipose-AS.M.Diab- | 11.7 | 139520_pancreas-CC.M.Norm-hi BMI-29 | 0.0 |
| hi BMI-9 | | | |
| 137836_subQadipose-HI.M.Diab.- | 20.3 | 139523_pancreas-HI.M.Norm-hi BMI-31 | 0.0 |
| hi BMI-21 | | | |
| 141340_subQadipose-AA.M.Diab.- | 20.2 | 142743_pancreas-AA.M.Norm-hi BMI-25 | 0.0 |
| hi BMI-6 | | | |
| 137831_vis.adipose-CC.M.Diab.- | 0.4 | 143546_small intestine-HI.M.Norm-low | 0.0 |
| low BMI-13 | | BMI-41 | |
| 139546_vis.adipose-HI.M.Diab.- | 23.5 | 143549_small intestine-CC.M.Norm-low | 0.0 |
| low BMI-22 | | BMI-39 | |
| 137839_vis.adipose-HI.M.Diab.- | 6.4 | 143550_small intestine-CC.M.Norm-low | 0.7 |
| med BMI-23 | | BMI-40 | |
| 137861_vis.adipose-CC.M.Diab.- | 19.1 | 143540_small intestine-CC.M.Norm-med | 0.0 |
| med BMI-1 | | BMI-26 | |
| 139510_vis.adipose-AA.M.Diab.- | 0.0 | 143547_small intestine-AA.M.Norm-med | 0.0 |
| med BMI-8 | | BMI-37 | |
| 143502_vis.adipose-CC.M.Diab.- | 80.1 | 143548_small intestine-AA.M.Norm-med | 0.0 |
| med BMI-2 | | BMI-47 | |
| 135759_vis.adipose-HI.M.Diab.-hi | 0.1 | 143539_small intestine-AA.M.Norm-hi | 0.0 |
| BMI-21 | | BMI-25 | |
| 135770_vis.adipose-AS.M.Diab-hi | 19.9 | 143542_small intestine-CC.M.Norm-hi | 0.0 |
| BMI-9 | | BMI-29 | |
| 137859_vis.adipose-AA.M.Diab.-hi | 0.1 | 143543_small intestine-HI.M.Norm-hi | 0.0 |
| BMI-6 | | BMI-31 | |
| 135758_liver-CC.M.Diab.-low | 0.0 | 143544_small intestine-AS.M.Norm-hi | 0.0 |
| BMI-13 | | BMI-34 | |
| 137838_liver-HI.M.Diab.-low BMI-22 | 0.0 | 97478_Patient-07pl (CC.Non-diab.obese) | 0.0 |
| 137827_liver-CC.M.Diab.-med | 0.0 | 97481_Patient-08sk (CC.Non-diab.obese) | 11.6 |
| BMI-1 | | | |
| 137840_liver-HI.M.Diab.-med | 0.0 | 110918_Patient-19go (CC.Non-diab.low | 41.5 |
| BMI-23 | | BMI) | |
| 139511_liver-AA.M.Diab.-med | 0.0 | 110921_Patient-19pl (CC.Non-diab.low | 100.0 |
| BMI-8 | | BMI) | |
| 139526_liver-CC.M.Diab.-med | 0.0 | 145435_Patient-21pl (CC.Non- | 0.0 |
| BMI-2 | | diab.overwt) | |
| 139514_liver-HI.M.Diab.-hi BMI- | 0.0 | 145443_Patient-23pl (CC.Non- | 0.1 |
| 21 | | diab.overwt) | |
| 141327_liver-CC.M.Diab.-hi BMI-4 | 0.0 | 97503_Patient-12pl (CC.Diab.unknown | 0.0 |
| | | BMI.insulin) | |
| 139513_pancreas-CC.M.Diab.-low | 0.0 | 145427_Patient-20pl | 0.0 |
| BMI-13 | | (CC.Diab.overwt.insulin) | |
| 142739_pancreas-AS.M.Diab.-low | 0.0 | 145438_Patient-22pl (CC.Diab.low | 0.0 |
| BMI-20 | | BMI.insulin) | |
| 139512_pancreas-AA.M.Diab.-med | 0.0 | 145441_Patient-22sk (CC.Diab.low | 5.5 |
| BMI-8 | | BMI.insulin) | |
| 139515_pancreas-HI.M.Diab.-med | 0.0 | 145461_Patient-26sk | 0.7 |
| BMI-23 | | (CC.Diab.obese.insulin) | |
| 139527_pancreas-CC.M.Diab.-med | 0.0 | 160111_Patient 27-go | 45.4 |
| BMI-1 | | (CC.Diab.obese.insulin) | |
| 141337_pancreas-CC.M.Diab.-med | 0.0 | 160112_Patient 27-sk | 0.8 |
| BMI-2 | | (CC.Diab.obese.insulin) | |
| 137837_pancreas-HI.M.Diab.-hi | 0.0 | 160113_Patient 27-pl | 0.0 |
| BMI-21 | | (CC.Diab.obese.insulin) | |
| 137856_pancreas-AA.M.Diab.-hi | 0.0 | 160114_Patient 27-ut | 0.0 |
| BMI-6 | | (CC.Diab.obese.insulin) | |
| 139525_pancreas-AS.M.Diab.-hi | 0.0 | 97828_Patient-13pl (CC.Diab.overwt.no | 0.0 |
| BMI-9 | | insulin) | |
| 141328_pancreas-CC.M.Diab.-hi | 0.0 | 100752_Patient-15sk (CC.Diab.obese.no | 0.9 |
| BMI-4 | | insulin) | |
| 143534_small intestine- | 0.0 | 110908_Patient-17go (CC.Diab.low | 46.3 |
| AA.M.Diab.-low BMI-17 | | BMI.no insulin) | |
| 143535_small intestine- | 0.0 | 110911_Patient-17pl (CC.Diab.low | 0.0 |
| AS.M.Diab.-low BMI-20 | | BMI.no insulin) | |
| 143537_small intestine-HI.M.Diab.- | 0.0 | 110913_Patient-18go (HI.Diab.obese.no | 30.4 |
| low BMI-22 | | insulin) | |
| 143528_small intestine- | 0.0 | 110916_Patient-18pl (HI.Diab.obese.no | 0.0 |
| CC.M.Diab.-med BMI-2 | | insulin) | |
| 143531_small intestine- | 0.0 | 145454_Patient-25pl (CC.Diab.low | 0.0 |
| AA.M.Diab.-med BMI-8 | | BMI.no insulin) | |
| 143538_small intestine-HI.M.Diab.- | 0.0 | 160117_Human Islets-obese male | 0.0 |
| med BMI-23 | | | |
| 143529_small intestine- | 0.0 | 145474_PANC1 (pancreas carcinoma) | 0.0 |
| CC.M.Diab.-hi BMI-4 | | | |
| 143530_small intestine- | 0.0 | 154911_Capan2 (pancreas | 0.0 |
| AA.M.Diab.-hi BMI-6 | | adenocarcinoma) | |
| 143512_hypothalamus- | 0.0 | 141190_SW579 (thyroid carcinoma) | 0.0 |
| CC.M.Norm-low BMI-40 | | | |
| 143521_hypothalamus- | 0.0 | 145489_SK-N-MC (neuroblastoma) | 0.0 |
| AS.M.Norm-low BMI-28 | | | |
| 143514_hypothalamus-HI.M.Norm- | 0.0 | 145495_SK-N-SH (neuroblastoma) | 0.0 |
| med BMI-35 | | | |
| 143517_hypothalamus- | 0.0 | 145498_U87 MG (glioblastoma) | 0.0 |
| AA.M.Norm-med BMI-47 | | | |
| 143504_hypothalamus- | 0.0 | 145484_HEp-2 (larynx carcinoma) | 0.0 |
| AA.M.Norm-hi BMI-25 | | | |
| 143511_hypothalamus- | 0.0 | 145479_A549 (lung carcinoma) | 0.0 |
| CC.M.Norm-hi BMI-29 | | | |
| 143516_hypothalamus- | 0.3 | 145488_A427 (lung carcinoma) | 0.0 |
| AS.M.Norm-hi BMI-34 | | | |
| 143522_hypothalamus-HI.M.Norm- | 0.0 | 145472_FHs 738Lu (normal lung) | 0.0 |
| hi BMI-31 | | | |
| 137874_diaphragm-AS.M.Norm- | 0.5 | 141187_SKW6.4 (B lymphocytes) | 0.0 |
| low BMI-28 | | | |
| 137877_diaphragm-CC.M.Norm- | 0.0 | 154644_IM-9 (immunoglobulin secreting | 0.0 |
| low BMI-39 | | lymphoblast) | |
| 139542_diaphragm-HI.M.Norm- | 0.1 | 154645_MOLT-4 (acute lymphoblastic | 0.0 |
| low BMI-41 | | leukemia) | |
| 135773_diaphragm-HI.M.Norm- | 0.4 | 154648_U-937 (histiocystic lymphoma) | 0.0 |
| med BMI-35 | | | |
| 137872_diaphragm-CC.M.Norm- | 0.1 | 154647_Daudi (Burkitt's lymphoma) | 0.0 |
| med BMI-26 | | | |
| 137879_diaphragm-AA.M.Norm- | 0.1 | 145494_SK-MEL-2 (melanoma) | 0.0 |
| med BMI-47 | | | |
| 137843_diaphragm-AA.M.Norm-hi | 0.0 | 141176_A375 (melanoma) | 0.0 |
| BMI-25 | | | |
| 137848_diaphragm-HI.M.Norm-hi | 0.4 | 154642_SW 1353 (humerus | 0.0 |
| BMI-31 | | chondrosarcoma) | |
| 139517_diaphragm-AS.M.Norm-hi | 0.1 | 141179_HT-1080 (fibrosarcoma) | 0.0 |
| BMI-34 | | | |
| 142738_diaphragm-CC.M.Norm-hi | 0.3 | 145491_MG-63 (osteosarcoma) | 0.0 |
| BMI-29 | | | |
| 135775_psoas-CC.M.Norm-low | 0.1 | 141186_MCF7 (breast carcinoma) | 0.0 |
| BMI-39 | | | |
| 137853_psoas-HI.M.Norm-low | 0.3 | 141193_T47D (breast carcinoma) | 0.0 |
| BMI-41 | | | |
| 137873_psoas-AS.M.Norm-low | 0.5 | 154641_BT-20 (breast carcinoma) | 0.0 |
| BMI-28 | | | |
| 142742_psoas-CC.M.Norm-low | 0.0 | 141175_293 (kidney transformed with | 0.0 |
| BMI-40 | | adenovirus 5 DNA) | |
| 137844_psoas-CC.M.Norm-med | 0.0 | 141182_HUH hepatoma | 0.0 |
| BMI-26 | | | |
| 137855_psoas-AA.M.Norm-med | 0.0 | 141184_HUH7 hepatoma | 0.0 |
| BMI-47 | | | |
| 142745_psoas-HI.M.Norm-med | 0.0 | 145478_HT1376 (bladder carcinoma) | 0.0 |
| BMI-35 | | | |
| 142746_psoas-AA.M.Norm-med | 0.0 | 145481_SCaBER (bladder carcinoma) | 0.0 |
| BMI-37 | | | |
| 135766_psoas-AA.M.Norm-hi | 0.0 | 141192_SW620 (lymph node metastatsis, | 0.0 |
| BMI-25 | | colon carcinoma) | |
| 135769_psoas-HI.M.Norm-hi BMI- | 0.2 | 141180_HT29 (colon Carcinoma) | 0.0 |
| 31 | | | |
| 137850_psoas-AS.M.Norm-hi BMI- | 0.1 | 141188_SW480 (colon carcinoma) | 0.0 |
| 34 | | | |
| 137876_subQadipose-CC.M.Norm- | 23.2 | 154646_CAOV-3 (ovary | 0.0 |
| low BMI-39 | | adenocarcinoma) | |
| 137878_subQadipose-HI.M.Norm- | 13.7 | 141194_HeLa (cervix carcinoma) | 0.0 |
| low BMI-41 | | | |
| 141331_subQadipose-AS.M.Norm- | 0.0 | 145482_HeLa S3 (cervix carcinoma) | 0.0 |
| low BMI-28 | | | |
| 137875_subQadipose-AA.M.Norm- | 9.5 | 145486_DU145 (prostate carcinoma) | 0.0 |
| med BMI-37 | | | |
| 139544_subQadipose-AA.M.Norm- | 66.9 | 154643_PC-3 (prostate adenocarcinoma) | 0.0 |
| med BMI-47 | | | |
| 141334_subQadipose-CC.M.Norm- | 0.0 | 154649_HCT-8 (ileocecal | 0.0 |
| med BMI-26 | | adenocarcinoma) | |
| 141339_subQadipose-HI.M.Norm- | 4.2 | | |
| med BMI-35 | | | |

General_screening_panel_v1.7 Summary: Ag7906 Highest expression of this gene is detected in adipose (CT=20.5). This gene codes for adeponectin (APM1). The APM1 gene is the most abundant transcript in adipose cells that regulates lipid metabolism in our body. It's primary role is to induce lipid catabolism in cells by inducing the expression of genes encoding CD36 (fatty acid transporter), acylcoA oxidase and uncoupling protein-2 (Saltiel, A. R.(2001) Nature Medicine 7, 887-888). This leads to an enhancement in fatty acid transport, fatty acid combustion, and energy dissipation. The net effect of adiponectin on cells is to decrease the accumulation of lipids in the body. In addition, by increasing fatty acid metabolism adiponectin may improve insulin receptor signaling by removing the attenuating effect of lipid on insulin-induced P13-kinase activity. It has been shown that injection of adiponectin in leptin deficient Ob/Ob mice dramatically normalizes blood sugar level without affecting the hyperinsulinemic condition in the mice suggesting that the mechanism of action of adiponectin may not involve the sensitization of the cells to the effect of insulin (Berg, A. H. et al. (2001) Nature Medicine 7, 947-953). The mechanism of action of adiponectin remains poorly understood. Using Curagen PathCalling technique, APM1 has been shown to interact with Calcium modulating ligand (CAMLG). The Calcium modulating ligand (CAMLG) functions in increasing intracellular calcium level by allowing entry of calcium from intracellular and extracellular sources (Gotz-Ulrich V. B. and Bram, R. J. (1997) Science 278, 138-141). Intracellular calcium signaling in adipocytes leads to lipogenesis, inhibition of lipolysis and differentiation of adipocytes leading to obesity (Shi H, Dirienzo, D. and Zemel M. B.(2001) FASEB J.15, 291-293). Thus, this interaction suggests that in line with the known functions of adiponectin in lipid metabolism, a novel function of adiponectin may be to inhibit CAMLG signaling on the membrane surface and prevent intracellular calcium build up. A decrease in the intracellular calcium level in adipocytes and muscle cells will initiate lipid breakdown and fatty acid metabolism. Therefore, the therapeutic intervention of CAMLG function on the membrane may prevent obesity and diabetes and complications associated with these diseases.

Moderate to low expression of this gene is also seen in number of tissues with metabolic/endocrine function including pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Human Metabolic Summary: Ag7906 Highest expression of this gene is detected in placenta of a non-diabetic normal weight patient (CT=22). Significant expression of this gene is also seen in adipose, pancreas, greater omentum and placenta from normal, obese and diabetic patients. Further utility for this gene is discussed under Panel 1.7.

C. CG92035-01: C1Q-Related Factor.

Expression of gene CG92035-01 was assessed using the primer-probe sets Ag3767, Ag4935 and Ag4902, described in Tables CA, CB and CC. Results of the RTQ-PCR runs are shown in Tables CD, CE, CF, CG, CH, CI, CJ and CK.

TABLE CA
Probe Name Ag3767

| | | | Start | SEQ ID |
|---|---|---|---|---|
| Primers | Sequences | Length | Position | No |
| Forward | 5′-gcggaccagaactacgactac-3′ | 21 | 652 | 33 |
| Probe | TET-5′-agcaacagcgtgatcctgcacct-3′-TAMRA | 23 | 676 | 34 |
| Reverse | 5′-tccatccagcttgatgaaga-3′ | 20 | 716 | 35 |

TABLE CB
Probe Name Ag4935

| | | | Start | SEQ ID |
|---|---|---|---|---|
| Primers | Sequences | Length | Position | No |
| Forward | 5′-gcggaccagaactacgactac-3′ | 21 | 652 | 36 |
| Probe | TET-5′-agcaacagcgtgatcctgcacct-3′-TAMRA | 23 | 676 | 37 |
| Reverse | 5′-tccatccagcttgatgaaga-3′ | 20 | 716 | 38 |

TABLE CC
Probe Name Ag4902

| | | | Start | SEQ ID |
|---|---|---|---|---|
| Primers | Sequences | Length | Position | No |
| Forward | 5′-gcggaccaqaactacgactac-3′ | 21 | 652 | 39 |
| Probe | TET-5′-agcaacagcgtgatcctgcacct-3′-TAMRA | 23 | 676 | 40 |
| Reverse | 5′-tccatccagcttgatgaaga-3′ | 20 | 716 | 41 |

TABLE CD
CNS_- neurodegeneration_v1.0
Column A - Rel. Exp.(%) Ag3767, Run 211176204
Column B - Rel. Exp.(%) Ag4902, Run 224996030
Column C - Rel. Exp.(%) Ag4935, Run 249286294

| Tissue Name | A | B | C | Tissue Name | A | B | C |
|---|---|---|---|---|---|---|---|
| AD 1 Hippo | 22.7 | 21.5 | 20.7 | Control (Path) 3 Temporal | 9.7 | 11.0 | 9.5 |
| | | | | Ctx | | | |
| AD 2 Hippo | 44.8 | 40.1 | 39.0 | Control (Path) 4 Temporal | 40.9 | 31.9 | 29.5 |
| | | | | Ctx | | | |
| AD 3 Hippo | 19.8 | 22.8 | 18.3 | AD 1 Occipital Ctx | 22.7 | 26.4 | 22.1 |
| AD 4 Hippo | 14.6 | 16.8 | 16.6 | AD 2 Occipital Ctx | 0.0 | 0.0 | 0.0 |
| | | | | (Misssing) | | | |
| AD 5 hippo | 73.2 | 70.7 | 63.3 | AD 3 Occipital Ctx | 14.6 | 15.2 | 12.0 |
| AD 6 Hippo | 100.0 | 100.0 | 80.1 | AD 4 Occipital Ctx | 28.1 | 30.4 | 26.4 |
| Control 2 Hippo | 33.2 | 40.9 | 32.1 | AD 5 Occipital Ctx | 17.8 | 18.3 | 15.6 |
| Control 4 Hippo | 42.3 | 47.3 | 35.8 | AD 6 Occipital Ctx | 42.6 | 51.4 | 38.7 |
| Control (Path) 3 Hippo | 9.1 | 11.1 | 10.7 | Control 1 Occipital Ctx | 8.7 | 7.1 | 10.5 |
| AD 1 Temporal Ctx | 32.3 | 39.2 | 33.9 | Control 2 Occipital Ctx | 46.3 | 51.8 | 45.7 |
| AD 2 Temporal Ctx | 41.8 | 51.8 | 40.3 | Control 3 Occipital Ctx | 27.5 | 24.5 | 22.1 |
| AD 3 Temporal Ctx | 17.3 | 16.7 | 14.6 | Control 4 Occipital Ctx | 18.8 | 17.6 | 17.6 |
| AD 4 Temporal Ctx | 30.1 | 31.0 | 34.6 | Control (Path) 1 Occipital | 79.0 | 74.2 | 77.4 |
| | | | | Ctx | | | |
| AD 5 Inf Temporal Ctx | 79.6 | 93.3 | 100.0 | Control (Path) 2 Occipital | 18.8 | 15.4 | 16.5 |
| | | | | Ctx | | | |
| AD 5 SupTemporal Ctx | 66.9 | 68.8 | 69.7 | Control (Path) 3 Occipital | 5.8 | 5.3 | 3.1 |
| | | | | Ctx | | | |
| AD 6 Inf Temporal Ctx | 60.3 | 67.4 | 66.0 | Control (Path) 4 Occipital | 27.7 | 24.7 | 29.9 |
| | | | | Ctx | | | |
| AD 6 Sup Temporal Ctx | 69.3 | 73.2 | 62.9 | Control 1 Parietal Ctx | 17.2 | 16.0 | 17.6 |
| Control 1 Temporal Ctx | 22.7 | 23.7 | 19.5 | Control 2 Parietal Ctx | 65.5 | 57.4 | 64.2 |
| Control 2 Temporal Ctx | 36.3 | 51.8 | 43.5 | Control 3 Parietal Ctx | 14.9 | 20.2 | 17.9 |
| Control 3 Temporal Ctx | 21.9 | 27.7 | 20.2 | Control (Path) 1 Parietal | 55.9 | 61.6 | 56.3 |
| | | | | Ctx | | | |
| Control 4 Temporal Ctx | 20.2 | 16.8 | 18.4 | Control (Path) 2 Parietal | 33.0 | 28.3 | 27.0 |
| | | | | Ctx | | | |
| Control (Path) 1 Temporal | 54.0 | 57.4 | 46.0 | Control (Path) 3 Parietal | 7.0 | 6.8 | 7.3 |
| Ctx | | | | Ctx | | | |
| Control (Path) 2 Temporal | 45.7 | 48.3 | 36.6 | Control (Path) 4 Parietal | 39.0 | 41.5 | 37.6 |
| Ctx | | | | Ctx | | | |

TABLE CE
General_screening_panel_v1.4
Column A - Rel. Exp.(%) Ag3767, Run 218981839

| Tissue Name | A | Tissue Name | A |
|---|---|---|---|
| Adipose | 0.2 | Renal ca.TK-10 | 0.6 |
| Melanoma* Hs688(A).T | 0.1 | Bladder | 0.3 |
| Melanoma* Hs688(B).T | 0.1 | Gastric ca. (liver met.) NCI-N87 | 0.1 |
| Melanoma* M14 | 0.6 | Gastric ca. KATO III | 0.0 |
| Melanoma* LOXIMVI | 0.0 | Colon ca. SW-948 | 0.0 |
| Melanoma* SK-MEL-5 | 2.0 | Colon ca. SW480 | 0.5 |
| Squamous cell carcinoma | 0.1 | Colon ca.* (SW480 met) SW620 | 0.0 |
| SCC-4 | | | |
| Testis Pool | 0.1 | Colon ca. HT29 | 0.0 |
| Prostate ca.* (bone | 1.8 | Colon ca. HCT-116 | 0.5 |
| met) PC-3 | | | |
| Prostate Pool | 0.3 | Colon ca. CaCo-2 | 0.6 |
| Placenta | 0.0 | Colon cancer tissue | 0.0 |
| Uterus Pool | 0.2 | Colon ca. SW1116 | 1.3 |
| Ovarian ca. OVCAR-3 | 0.2 | Colon ca. Colo-205 | 0.0 |
| Ovarian ca. SK-OV-3 | 14.2 | Colon ca. SW-48 | 0.0 |
| Ovarian ca. OVCAR-4 | 0.1 | Colon Pool | 0.1 |
| Ovarian ca. OVCAR-5 | 2.2 | Small Intestine Pool | 0.3 |
| Ovarian ca. IGROV-1 | 4.0 | Stomach Pool | 0.2 |
| Ovarian ca. OVCAR-8 | 8.9 | Bone Marrow Pool | 0.2 |
| Ovary | 0.1 | Fetal Heart | 1.9 |
| Breast ca. MCF-7 | 1.7 | Heart Pool | 0.3 |
| Breast ca. MDA-MB-231 | 0.7 | Lymph Node Pool | 0.4 |
| Breast ca. BT 549 | 7.9 | Fetal Skeletal Muscle | 0.2 |
| Breast ca. T47D | 10.0 | Skeletal Muscle Pool | 0.0 |
| Breast ca. MDA-N | 0.1 | Spleen Pool | 0.0 |
| Breast Pool | 0.1 | Thymus Pool | 0.1 |
| Trachea | 0.2 | CNS cancer (glio/astro) | 1.2 |
| | | U87-MG | |
| Lung | 0.0 | CNS cancer (glio/astro) | 3.3 |
| | | U-118-MG | |
| Fetal Lung | 0.4 | CNS cancer (neuro;met) | 2.0 |
| | | SK-N-AS | |
| Lung ca. NCI-N417 | 1.1 | CNS cancer (astro) SF-539 | 1.0 |
| Lung ca. LX-1 | 0.0 | CNS cancer (astro) SNB-75 | 11.0 |
| Lung ca. NCI-H146 | 4.9 | CNS cancer (glio) SNB-19 | 3.3 |
| Lung ca. SHP-77 | 4.2 | CNS cancer (glio) SF-295 | 5.0 |
| Lung ca. A549 | 2.1 | Brain (Amygdala) Pool | 1.1 |
| Lung ca. NCI-H526 | 0.3 | Brain (cerebellum) | 1.8 |
| Lung ca. NCI-H23 | 1.7 | Brain (fetal) | 1.9 |
| Lung ca. NCI-H460 | 0.2 | Brain (Hippocampus) Pool | 1.8 |
| Lung ca. HOP-62 | 3.1 | Cerebral Cortex Pool | 2.0 |
| Lung ca. NCI-H522 | 1.8 | Brain (Substantia nigra) Pool | 2.4 |
| Liver | 0.0 | Brain (Thalamus) Pool | 2.3 |
| Fetal Liver | 0.1 | Brain (whole) | 2.5 |
| Liver ca. HepG2 | 0.0 | Spinal Cord Pool | 4.0 |
| Kidney Pool | 1.0 | Adrenal Gland | 0.2 |
| Fetal Kidney | 0.4 | Pituitary gland Pool | 0.0 |
| Renal ca. 786-0 | 34.6 | Salivary Gland | 0.0 |
| Renal ca. A498 | 100.0 | Thyroid (female) | 0.1 |
| Renal ca. ACHN | 4.3 | Pancreatic ca. CAPAN2 | 0.1 |
| Renal ca. UO-31 | 0.2 | Pancreas Pool | 0.3 |

TABLE CF
General_screening_panel_v1.5
Column A - Rel. Exp.(%) Ag4935, Run 228850845

| Tissue Name | A | Tissue Name | A |
|---|---|---|---|
| Adipose | 0.2 | Renal ca. TK-10 | 0.8 |
| Melanoma* Hs688(A).T | 0.1 | Bladder | 0.3 |
| Melanoma* Hs688(B).T | 0.1 | Gastric ca. (liver met.) NCI-N87 | 0.1 |
| Melanoma* M14 | 0.8 | Gastric ca. KATO III | 0.0 |
| Melanoma* LOXIMVI | 0.0 | Colon ca. SW-948 | 0.0 |
| Melanoma* SK-MEL-5 | 2.1 | Colon ca. SW480 | 1.1 |
| Squamous cell carcinoma | 0.2 | Colon ca.* (SW480 met) SW620 | 0.1 |
| SCC-4 | | | |
| Testis Pool | 0.2 | Colon ca. HT29 | 0.0 |
| Prostate ca.* (bone | 0.6 | Colon ca. HCT-116 | 0.7 |
| met) PC-3 | | | |
| Prostate Pool | 0.3 | Colon ca. CaCo-2 | 0.8 |
| Placenta | 0.0 | Colon cancer tissue | 0.1 |
| Uterus Pool | 0.3 | Colon ca. SW1116 | 1.2 |
| Ovarian ca. OVCAR-3 | 0.2 | Colon ca. Colo-205 | 0.0 |
| Ovarian ca. SK-OV-3 | 15.5 | Colon ca. SW-48 | 0.0 |
| Ovarian ca. OVCAR-4 | 0.2 | Colon Pool | 0.3 |
| Ovarian ca. OVCAR-5 | 3.7 | Small Intestine Pool | 0.2 |
| Ovarian ca. IGROV-1 | 6.4 | Stomach Pool | 0.1 |
| Ovarian ca. OVCAR-8 | 12.4 | Bone Marrow Pool | 0.3 |
| Ovary | 0.1 | Fetal Heart | 2.6 |
| Breast ca. MCF-7 | 1.8 | Heart Pool | 0.5 |
| Breast ca. MDA-MB-231 | 0.8 | Lymph Node Pool | 0.5 |
| Breast ca. BT 549 | 7.7 | Fetal Skeletal Muscle | 0.2 |
| Breast ca. T47D | 0.5 | Skeletal Muscle Pool | 0.0 |
| Breast ca. MDA-N | 0.1 | Spleen Pool | 0.0 |
| Breast Pool | 0.1 | Thymus Pool | 0.1 |
| Trachea | 0.1 | CNS cancer (glio/astro) | 1.8 |
| | | U87-MG | |
| Lung | 0.0 | CNS cancer (glio/astro) | 3.3 |
| | | U-118-MG | |
| Fetal Lung | 0.5 | CNS cancer (neuro;met) | 2.2 |
| | | SK-N-AS | |
| Lung ca. NCI-N417 | 1.3 | CNS cancer (astro) SF-539 | 1.1 |
| Lung ca. LX-1 | 0.1 | CNS cancer (astro) SNB-75 | 12.4 |
| Lung ca. NCI-H146 | 5.8 | CNS cancer (glio) SNB-19 | 4.8 |
| Lung ca. SHP-77 | 6.3 | CNS cancer (glio) SF-295 | 5.2 |
| Lung ca. A549 | 3.5 | Brain (Amygdala) Pool | 1.7 |
| Lung ca. NCI-H526 | 0.3 | Brain (cerebellum) | 2.7 |
| Lung ca. NCI-H23 | 1.9 | Brain (fetal) | 3.2 |
| Lung ca. NCI-H460 | 0.2 | Brain (Hippocampus) Pool | 3.0 |
| Lung ca. HOP-62 | 4.3 | Cerebral Cortex Pool | 2.3 |
| Lung ca. NCI-H522 | 3.7 | Brain (Substantia nigra) Pool | 2.2 |
| Liver | 0.0 | Brain (Thalamus) Pool | 3.0 |
| Fetal Liver | 0.2 | Brain (whole) | 4.0 |
| Liver ca. HepG2 | 0.0 | Spinal Cord Pool | 3.5 |
| Kidney Pool | 1.4 | Adrenal Gland | 0.1 |
| Fetal Kidney | 0.5 | Pituitary gland Pool | 0.0 |
| Renal ca. 786-0 | 40.9 | Salivary Gland | 0.0 |
| Renal ca. A498 | 100.0 | Thyroid (female) | 0.1 |
| Renal ca. ACHN | 5.4 | Pancreatic ca. CAPAN2 | 0.1 |
| Renal ca. UO-31 | 0.3 | Pancreas Pool | 0.3 |

TABLE CG
HASS Panel v1.0
Column A - Rel. Exp.(%) Ag3767, Run 318641613

| Tissue Name | A | Tissue Name | A |
|---|---|---|---|
| MCF-7 C1 | 1.2 | U87-MG F1 (B) | 4.8 |
| MCF-7 C2 | 1.1 | U87-MG F2 | 7.8 |
| MCF-7 C3 | 0.6 | U87-MG F3 | 11.2 |
| MCF-7 C4 | 1.9 | U87-MG F4 | 12.8 |
| MCF-7 C5 | 0.9 | U87-MG F5 | 14.2 |
| MCF-7 C6 | 1.1 | U87-MG F6 | 20.2 |
| MCF-7 C7 | 0.9 | U87-MG F7 | 21.9 |
| MCF-7 C9 | 0.8 | U87-MG F8 | 26.8 |
| MCF-7 C10 | 2.9 | U87-MG F9 | 25.7 |
| MCF-7 C11 | 0.4 | U87-MG F10 | 21.8 |
| MCF-7 C12 | 0.8 | U87-MG F11 | 27.5 |
| MCF-7 C13 | 0.8 | U87-MG F12 | 26.4 |
| MCF-7 C15 | 0.4 | U87-MG F13 | 56.6 |
| MCF-7 C16 | 1.6 | U87-MG F14 | 58.2 |
| MCF-7 C17 | 1.8 | U87-MG F15 | 78.5 |
| T24 D1 | 1.3 | U87-MG F16 | 19.9 |
| T24 D2 | 1.6 | U87-MG F17 | 27.0 |
| T24 D3 | 2.2 | LnCAP A1 | 0.3 |
| T24 D4 | 3.2 | LnCAP A2 | 0.2 |
| T24 D5 | 1.8 | LnCAP A3 | 0.4 |
| T24 D6 | 1.4 | LnCAP A4 | 0.1 |
| T24 D7 | 4.7 | LnCAP A5 | 0.3 |
| T24 D9 | 8.8 | LnCAP A6 | 0.3 |
| T24 D10 | 3.3 | LnCAP A7 | 0.4 |
| T24 D11 | 2.7 | LnCAP A8 | 0.8 |
| T24 D12 | 2.0 | LnCAP A9 | 0.5 |
| T24 D13 | 12.5 | LnCAP A10 | 1.0 |
| T24 D15 | 6.7 | LnCAP A11 | 0.9 |
| T24 D16 | 0.2 | LnCAP A12 | 0.2 |
| T24 D17 | 0.4 | LnCAP A13 | 0.3 |
| CAPaN B1 | 0.4 | LnCAP A14 | 0.4 |
| CAPaN B2 | 0.2 | LnCAP A15 | 0.3 |
| CAPaN B3 | 0.1 | LnCAP A16 | 0.4 |
| CAPaN B4 | 0.2 | LnCAP A17 | 0.5 |
| CAPaN B5 | 0.2 | Primary Astrocytes | 1.7 |
| CAPaN B6 | 0.1 | Primary Renal Proximal | 0.0 |
| | | Tubule Epithelial cell A2 | |
| CAPaN B7 | 0.2 | Primary melanocytes A5 | 0.1 |
| CAPaN B8 | 0.1 | 126443-341 medullo | 0.2 |
| CAPaN B9 | 0.1 | 126444-487 medullo | 3.2 |
| CAPaN B10 | 0.4 | 126445-425 medullo | 0.0 |
| CAPaN B11 | 0.2 | 126446-690 medullo | 0.8 |
| CAPaN B12 | 0.0 | 126447-54 adult glioma | 12.9 |
| CAPaN B13 | 0.2 | 126448-245 adult glioma | 24.7 |
| CAPaN B14 | 0.1 | 126449-317 adult glioma | 4.6 |
| CAPaN B15 | 100.0 | 126450-212 glioma | 54.7 |
| CAPaN B16 | 0.0 | 126451-456 glioma | 9.3 |
| CAPaN B17 | 0.1 | | |

TABLE CH
Oncology_cell_line_screening_panel_v3.2
Column A - Rel. Exp.(%) Ag3767, Run 259496875

| Tissue Name | A | Tissue Name | A |
|---|---|---|---|
| 94905_Daoy_Medulloblastoma/Cerebellum_{—} | 0.7 | 94954_Ca Ski_Cervical epidermoid | 0.8 |
| sscDNA | | carcinoma (metastasis)_sscDNA | |
| 94906_TE671_Medulloblastom/Cerebellum_{—} | 10.7 | 94955_ES-2_Ovarian clear cell | 0.1 |
| sscDNA | | carcinoma_sscDNA | |
| 94907_D283 | 1.5 | 94957_Ramos/6h stim_Stimulated | 0.0 |
| Med_Medulloblastoma/Cerebellum_sscDNA | | with PMA/ionomycin 6h_sscDNA | |
| 94908_PFSK-1_Primitive | 8.3 | 94958_Ramos/14h stim_Stimulated | 0.0 |
| Neuroectodermal/Cerebellum_sscDNA | | with PMA/ionomycin 14h_sscDNA | |
| 94909_XF-498_CNS_sscDNA | 2.6 | 94962_MEG-01_Chronic | 0.0 |
| | | myelogenous leukemia | |
| | | (megokaryoblast)_sscDNA | |
| 94910_SNB-78_CNS/glioma_sscDNA | 0.7 | 94963_Raji_Burkitt's | 0.0 |
| | | lymphoma_sscDNA | |
| 94911_SF-268_CNS/glioblastoma_sscDNA | 11.9 | 94964_Daudi_Burkitt's | 0.0 |
| | | lymphoma_sscDNA | |
| 94912_T98G_Glioblastoma_sscDNA | 8.2 | 94965_U266_B-cell | 0.0 |
| | | plasmacytoma/myeloma_sscDNA | |
| 96776_SK-N-SH_Neuroblastoma | 3.4 | 94968_CA46_Burkitt's | 0.0 |
| (metastasis)_sscDNA | | lymphoma_sscDNA | |
| 94913_SF-295_CNS/glioblastoma_sscDNA | 3.3 | 94970_RL_non-Hodgkin's B-cell | 0.0 |
| | | lymphoma_sscDNA | |
| 132565_NT2 pool_sscDNA | 2.7 | 94972_JM1_pre-B-cell | 0.0 |
| | | lymphoma/leukemia_sscDNA | |
| 94914_Cerebellum_sscDNA | 3.9 | 94973_Jurkat_T cell | 6.6 |
| | | leukemia_sscDNA | |
| 96777_Cerebellum_sscDNA | 4.3 | 94974_TF- | 0.1 |
| | | 1_Erythroleukemia_sscDNA | |
| 94916_NCI-H292_Mucoepidermoid lung | 0.6 | 94975_HUT 78_T-cell | 0.5 |
| carcinoma_sscDNA | | lymphoma_sscDNA | |
| 94917_DMS-114_Small cell lung | 4.2 | 94977_U937_Histiocytic | 0.2 |
| cancer_sscDNA | | lymphoma_sscDNA | |
| 94918_DMS-79_Small cell lung | 53.6 | 94980_KU-812_Myelogenous | 0.0 |
| cancer/neuroendocrine_sscDNA | | leukemia_sscDNA | |
| 94919_NCI-H146_Small cell lung | 17.3 | 94981_769-P_Clear cell renal | 1.0 |
| cancer/neuroendocrine_sscDNA | | carcinoma_sscDNA | |
| 94920_NCI-H526_Small cell lung | 1.7 | 94983_Caki-2_Clear cell renal | 3.3 |
| cancer/neuroendocrine_sscDNA | | carcinoma_sscDNA | |
| 94921_NCI-N417_Small cell lung | 5.4 | 94984_SW 839_Clear cell renal | 100.0 |
| cancer/neuroendocrine_sscDNA | | carcinoma_sscDNA | |
| 94923_NCI-H82_Small cell lung | 3.1 | 94986_G401_Wilms' | 10.2 |
| cancer/neuroendocrine_sscDNA | | tumor_sscDNA | |
| 94924_NCI-H157_Squamous cell lung | 1.1 | 126768_293 cells_sscDNA | 0.9 |
| cancer (metastasis)_sscDNA | | | |
| 94925_NCI-H1155_Large cell lung | 17.7 | 94987_Hs766T_Pancreatic | 0.7 |
| cancer/neuroendocrine_sscDNA | | carcinoma (LN metastasis)_sscDNA | |
| 94926_NCI-H1299_Large cell lung | 7.6 | 94988_CAPAN-1_Pancreatic | 0.1 |
| cancer/neuroendocrine_sscDNA | | adenocarcinoma (liver | |
| | | metastasis)_sscDNA | |
| 94927_NCI-H727_Lung carcinoid_sscDNA | 3.5 | 94989_SU86.86_Pancreatic | 0.6 |
| | | carcinoma (liver | |
| | | metastasis)_sscDNA | |
| 94928_NCI-UMC-11_Lung | 0.8 | 94990_BxPC-3_Pancreatic | 0.1 |
| carcinoid sscDNA | | adenocarcinoma_sscDNA | |
| 94929_LX-1_Small cell lung | 0.0 | 94991_HPAC_Pancreatic | 0.0 |
| cancer_sscDNA | | adenocarcinoma_sscDNA | |
| 94930_Colo-205_Colon cancer_sscDNA | 0.0 | 94992_MIA PaCa-2_Pancreatic | 0.4 |
| | | carcinoma_sscDNA | |
| 94931_KM12_Colon cancer_sscDNA | 0.0 | 94993_CFPAC-1_Pancreatic ductal | 0.1 |
| | | adenocarcinoma_sscDNA | |
| 94932_KM20L2_Colon cancer_sscDNA | 0.0 | 94994_PANC-1_Pancreatic | 2.5 |
| | | epithelioid ductal | |
| | | carcinoma_sscDNA | |
| 94933_NCI-H716_Colon cancer_sscDNA | 0.7 | 94996_T24_Bladder carcinma | 1.7 |
| | | (transitional cell)_sscDNA | |
| 94935_SW-48_Colon | 0.0 | 94997_5637_Bladder | 0.3 |
| adenocarcinoma_sscDNA | | carcinoma_sscDNA | |
| 94936_SW1116_Colon | 3.1 | 94998_HT-1197_Bladder | 0.1 |
| adenocarcinoma_sscDNA | | carcinoma_sscDNA | |
| 94937_LS 174T_Colon | 0.0 | 94999_UM-UC-3_Bladder carcinma | 2.3 |
| adenocarcinoma_sscDNA | | (transitional cell)_sscDNA | |
| 94938_SW-948_Colon | 0.0 | 95000_A204_Rhabdomyosarcoma_sscDNA | 3.1 |
| adenocarcinoma_sscDNA | | | |
| 94939_SW-480_Colon | 0.1 | 95001_HT- | 4.4 |
| adenocarcinoma_sscDNA | | 1080_Fibrosarcoma_sscDNA | |
| 94940_NCI-SNU-5_Gastric | 0.9 | 95002_MG-63_Osteosarcoma | 7.9 |
| carcinoma_sscDNA | | (bone)_sscDNA | |
| 112197_KATO III_Stomach_sscDNA | 0.0 | 95003_SK-LMS-1_Leiomyosarcoma | 0.8 |
| | | (vulva)_sscDNA | |
| 94943_NCI-SNU-16_Gastric | 0.0 | 95004_SJRH30_Rhabdomyosarcoma | 0.0 |
| carcinoma_sscDNA | | (met to bone marrow)_sscDNA | |
| 94944_NCI-SNU-1_Gastric | 0.1 | 95005_A431_Epidermoid | 0.5 |
| carcinoma_sscDNA | | carcinoma_sscDNA | |
| 94946_RF-1_Gastric | 0.0 | 95007_WM266- | 2.0 |
| adenocarcinoma_sscDNA | | 4_Melanoma_sscDNA | |
| 94947_RF-48_Gastric | 0.0 | 112195_DU 145_Prostate_sscDNA | 6.0 |
| adenocarcinoma_sscDNA | | | |
| 96778_MKN-45_Gastric | 0.2 | 95012_MDA-MB-468_Breast | 0.4 |
| carcinoma_sscDNA | | adenocarcinoma_sscDNA | |
| 94949_NCI-N87_Gastric | 0.1 | 112196_SSC-4_Tongue_sscDNA | 0.1 |
| carcinoma_sscDNA | | | |
| 94951_OVCAR-5_Ovarian | 0.3 | 112194_SSC-9_Tongue_sscDNA | 0.5 |
| carcinoma_sscDNA | | | |
| 94952_RL95-2_Uterine carcinoma_sscDNA | 2.0 | 112191_SSC-15_Tongue_sscDNA | 0.2 |
| 94953_HelaS3_Cervical | 5.0 | 95017_CAL 27_Squamous cell | 0.7 |
| adenocarcinoma_sscDNA | | carcinoma of tongue_sscDNA | |

TABLE CI
Panel 4.1D
Column A - Rel. Exp.(%) Ag3767, Run 170069112
Column B - Rel. Exp.(%) Ag4935, Run 223598631

| Tissue Name | A | B | Tissue Name | A | B |
|---|---|---|---|---|---|
| Secondary Th1 act | 0.0 | 0.5 | HUVEC IL-1beta | 3.3 | 2.3 |
| Secondary Th2 act | 4.0 | 1.4 | HUVEC IFN gamma | 2.9 | 4.0 |
| Secondary Tr1 act | 4.5 | 0.5 | HUVEC TNF alpha + IFN gamma | 1.4 | 1.2 |
| Secondary Th1 rest | 0.0 | 0.6 | HUVEC TNF alpha + IL4 | 0.7 | 1.1 |
| Secondary Th2 rest | 2.9 | 2.5 | HUVEC IL-11 | 3.2 | 1.8 |
| Secondary Tr1 rest | 2.9 | 0.0 | Lung Microvascular EC none | 5.6 | 2.0 |
| Primary Th1 act | 2.3 | 0.3 | Lung Microvascular EC TNFalpha + | 5.8 | 2.9 |
| | | | IL-1beta | | |
| Primary Th2 act | 0.0 | 0.3 | Microvascular Dermal EC none | 1.5 | 0.8 |
| Primary Tr1 act | 3.5 | 0.6 | Microsvasular Dermal EC | 0.0 | 1.1 |
| | | | TNFalpha + IL-1beta | | |
| Primary Th1 rest | 0.7 | 0.6 | Bronchial epithelium TNFalpha + | 1.8 | 1.9 |
| | | | IL1beta | | |
| Primary Th2 rest | 0.0 | 0.3 | Small airway epithelium none | 0.0 | 0.0 |
| Primary Tr1 rest | 3.0 | 2.9 | Small airway epithelium TNFalpha + | 1.7 | 0.2 |
| | | | IL-1beta | | |
| CD45RA CD4 lymphocyte | 9.1 | 3.6 | Coronery artery SMC rest | 2.7 | 0.8 |
| act | | | | | |
| CD45RO CD4 lymphocyte | 1.2 | 0.0 | Coronery artery SMC TNFalpha + | 0.0 | 0.9 |
| act | | | IL-1beta | | |
| CD8 lymphocyte act | 0.6 | 0.0 | Astrocytes rest | 100.0 | 43.8 |
| Secondary CD8 lymphocyte | 0.6 | 0.3 | Astrocytes TNFalpha + IL-1beta | 53.2 | 15.8 |
| rest | | | | | |
| Secondary CD8 lymphocyte | 1.5 | 1.5 | KU-812 (Basophil) rest | 0.0 | 1.4 |
| act | | | | | |
| CD4 lymphocyte none | 0.8 | 0.0 | KU-812 (Basophil) PMA/ionomycin | 0.0 | 1.1 |
| 2ry Th1/Th2/Tr1_anti-CD95 | 1.7 | 0.0 | CCD1106 (Keratinocytes) none | 23.5 | 8.5 |
| CH11 | | | | | |
| LAK cells rest | 6.7 | 1.2 | CCD1106 (Keratinocytes) | 8.5 | 5.4 |
| | | | TNFalpha + IL-1beta | | |
| LAK cells IL-2 | 2.8 | 1.6 | Liver cirrhosis | 6.8 | 4.2 |
| LAK cells IL-2+IL-12 | 2.4 | 0.0 | NCI-H292 none | 22.2 | 13.7 |
| LAK cells IL-2+IFN gamma | 3.1 | 0.3 | NCI-H292 IL-4 | 49.7 | 19.8 |
| LAK cells IL-2+IL-18 | 8.7 | 1.3 | NCI-H292 IL-9 | 79.0 | 43.5 |
| LAK cells PMA/ionomycin | 3.3 | 2.7 | NCI-H292 IL-13 | 50.0 | 40.6 |
| NK Cells IL-2 rest | 25.5 | 12.4 | NCI-H292 IFN gamma | 66.9 | 31.2 |
| Two Way MLR 3 day | 0.7 | 0.0 | HPAEC none | 6.7 | 1.5 |
| Two Way MLR 5 day | 0.8 | 0.7 | HPAEC TNF alpha + IL-1beta | 5.0 | 1.6 |
| Two Way MLR 7 day | 0.7 | 0.0 | Lung fibroblast none | 62.0 | 36.9 |
| PBMC rest | 0.0 | 0.0 | Lung fibroblast TNF alpha + IL- | 42.3 | 31.4 |
| | | | 1beta | | |
| PBMC PWM | 0.0 | 0.0 | Lung fibroblast IL-4 | 35.1 | 35.1 |
| PBMC PHA-L | 14.9 | 9.2 | Lung fibroblast IL-9 | 59.9 | 26.1 |
| Ramos (B cell) none | 0.0 | 0.0 | Lung fibroblast IL-13 | 48.0 | 20.0 |
| Ramos (B cell) ionomycin | 0.0 | 0.0 | Lung fibroblast IFN gamma | 88.9 | 44.4 |
| B lymphocytes PWM | 0.0 | 1.1 | Dermal fibroblast CCD1070 rest | 14.5 | 6.0 |
| B lymphocytes CD40L and | 0.0 | 0.4 | Dermal fibroblast CCD1070 TNF | 16.8 | 6.3 |
| IL-4 | | | alpha | | |
| EOL-1 dbcAMP | 0.0 | 0.0 | Dermal fibroblast CCD1070 IL- | 10.8 | 6.6 |
| | | | 1beta | | |
| EOL-1 dbcAMP | 0.0 | 0.0 | Dermal fibroblast IFN gamma | 21.5 | 8.8 |
| PMA/ionomycin | | | | | |
| Dendritic cells none | 0.0 | 0.0 | Dermal fibroblast IL-4 | 19.2 | 17.1 |
| Dendritic cells LPS | 0.0 | 0.4 | Dermal Fibroblasts rest | 13.5 | 7.9 |
| Dendritic cells anti-CD40 | 0.0 | 0.0 | Neutrophils TNFa+LPS | 0.0 | 0.6 |
| Monocytes rest | 0.0 | 0.0 | Neutrophils rest | 0.0 | 1.5 |
| Monocytes LPS | 0.0 | 0.0 | Colon | 24.8 | 7.5 |
| Macrophages rest | 0.0 | 0.0 | Lung | 5.4 | 7.4 |
| Macrophages LPS | 0.0 | 0.9 | Thymus | 3.0 | 22.1 |
| HUVEC none | 1.7 | 0.0 | Kidney | 44.8 | 100.0 |
| HUVEC starved | 1.8 | 1.8 | | | |

TABLE CJ
Panel 5 Islet
Column A - Rel. Exp. (%) Ag4935, Run 264532593

| Tissue Name | A | Tissue Name | A |
|---|---|---|---|
| 97457_Patient-02go_adipose | 5.1 | 94709_Donor 2 AM —A_adipose | 0.2 |
| 97476_Patient-07sk_skeletal muscle | 0.0 | 94710_Donor 2 AM —B_adipose | 0.2 |
| 97477_Patient-07ut_uterus | 0.7 | 94711_Donor 2 AM —C_adipose | 0.4 |
| 97478_Patient-07pl_placenta | 0.1 | 94712_Donor 2 AD —A_adipose | 0.4 |
| 99167_Bayer Patient 1 | 100.0 | 94713_Donor 2 AD —B_adipose | 0.3 |
| 97482_Patient-08ut_uterus | 0.5 | 94714_Donor 2 AD —C_adipose | 0.3 |
| 97483_Patient-08pl_placenta | 0.0 | 94742_Donor 3 U —A_Mesenchymal | 0.1 |
| | | Stem Cells | |
| 97486_Patient-09sk_skeletal muscle | 0.0 | 94743_Donor 3 U —B_Mesenchymal | 0.0 |
| | | Stem Cells | |
| 97487_Patient-09ut_uterus | 0.8 | 94730_Donor 3 AM —A_adipose | 0.3 |
| 97488_Patient-09pl_placenta | 0.1 | 94731_Donor 3 AM —B_adipose | 0.3 |
| 97492_Patient-10ut_uterus | 1.8 | 94732_Donor 3 AM —C_adipose | 0.4 |
| 97493_Patient-10pl_placenta | 0.0 | 94733_Donor 3 AD —A_adipose | 0.2 |
| 97495_Patient-11go_adipose | 5.5 | 94734_Donor 3 AD —B_adipose | 0.3 |
| 97496_Patient-11sk_skeletal muscle | 0.0 | 94735_Donor 3 AD —C_adipose | 0.1 |
| 97497_Patient-11ut_uterus | 3.0 | 77138_Liver_HepG2untreated | 1.8 |
| 97498_Patient-11pl_placenta | 0.0 | 73556_Heart_Cardiac stromal cells | 0.3 |
| | | (primary) | |
| 97500_Patient-12go_adipose | 5.1 | 81735_Small Intestine | 3.0 |
| 97501_Patient-12sk_skeletal muscle | 0.3 | 72409_Kidney_Proximal Convoluted | 0.1 |
| | | Tubule | |
| 97502_Patient-12ut_uterus | 1.2 | 82685_Small intestine_Duodenum | 0.9 |
| 97503_Patient-12pl_placenta | 0.1 | 90650_Adrenal_Adrenocortical adenoma | 0.0 |
| 94721_Donor 2 U —A_Mesenchymal | 0.3 | 72410_Kidney_HRCE | 0.3 |
| Stem Cells | | | |
| 94722_Donor 2 U —B_Mesenchymal | 0.1 | 72411_Kidney_HRE | 0.3 |
| Stem Cells | | | |
| 94723_Donor 2 U —C_Mesenchymal | 0.1 | 73139_Uterus_Uterine smooth muscle | 0.5 |
| Stem Cells | | cells | |

TABLE CK
general oncology screening panel_v_2.4
Column A - Rel. Exp.(%) Ag3767, Run 260268675

| Tissue Name | A | Tissue Name | A |
|---|---|---|---|
| Colon cancer 1 | 0.2 | Bladder NAT 2 | 0.0 |
| Colon NAT 1 | 0.5 | Bladder NAT 3 | 0.0 |
| Colon cancer 2 | 0.4 | Bladder NAT 4 | 0.5 |
| Colon NAT 2 | 0.4 | Prostate adenocarcinoma 1 | 18.0 |
| Colon cancer 3 | 0.7 | Prostate adenocarcinoma 2 | 0.5 |
| Colon NAT 3 | 0.8 | Prostate adenocarcinoma 3 | 0.2 |
| Colon malignant cancer 4 | 0.1 | Prostate adenocarcinoma 4 | 0.0 |
| Colon NAT 4 | 0.3 | Prostate NAT 5 | 0.4 |
| Lung cancer 1 | 0.3 | Prostate adenocarcinoma 6 | 0.2 |
| Lung NAT 1 | 0.0 | Prostate adenocarcinoma 7 | 0.6 |
| Lung cancer 2 | 0.4 | Prostate adenocarcinoma 8 | 0.2 |
| Lung NAT 2 | 0.0 | Prostate adenocarcinoma 9 | 32.8 |
| Squamous cell carcinoma 3 | 0.3 | Prostate NAT 10 | 0.1 |
| Lung NAT 3 | 0.0 | Kidney cancer 1 | 12.5 |
| Metastatic melanoma 1 | 0.2 | Kidney NAT 1 | 0.4 |
| Melanoma 2 | 0.0 | Kidney cancer 2 | 100.0 |
| Melanoma 3 | 0.1 | Kidney NAT 2 | 1.3 |
| Metastatic melanoma 4 | 1.0 | Kidney cancer 3 | 30.8 |
| Metastatic melanoma 5 | 0.4 | Kidney NAT 3 | 0.6 |
| Bladder cancer 1 | 0.1 | Kidney cancer 4 | 0.4 |
| Bladder NAT 1 | 0.0 | Kidney NAT 4 | 0.4 |
| Bladder cancer 2 | 0.8 | | |

CNS_neurodegeneration_v1.0 Summary: Ag3767/Ag4902/Ag4935 Three experiment with same probe-primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3767 High expression of this gene is seen in 2 renal cancer A498 and 786-0 cell lines (CTs=26-28). Therefore, expression of this gene may be used as diagnostic marker to detect the presence of renal/kidney cancer and also, therapeutic modulation of this gene through the use of antibodies or small molecule drug may be useful in the treatment of kidney cancer.

Moderate expression of this gene is also seen in number of cancer cell lines derived from melanoma, brain, breast, ovary, lung and prostate cancers. Therefore, expression of this gene may be used as diagnostic marker to detect the presence of these cancers.

Furthermore, therapeutic modulation of this gene or its protein product may be useful in the treatment of melanoma, brain, breast, ovary, lung and prostate cancers.

In addition, this gene is expressed at moderate to low levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CT=32.3) when compared to adult heart(CT=35). This observation suggests that expression of this gene can be used to distinguish fetal from adult heart. In addition, the relative overexpression of this gene in fetal tissue suggests that the protein product may enhance heart growth or development in the fetus and thus may also act in a regenerative capacity in the adult. Therefore, therapeutic modulation of the protein encoded by this gene could be useful in treatment of heart related diseases.

General_screening_panel_v1.5 Summary: Ag3767 High expression of this gene is seen in 2 renal cancer A498 and 786-0 cell lines (CTs=22-24). Expression profile seen in this panel correlates with that seen in panel 1.4, please see panel 1.4 for further discussion on the utility of this gene.

HASS Panel v1.0 Summary: Ag3767 Expression of this gene is slightly induced by treating U87 cells with a combination of reduced oxygen tension and acidic ph which suggests that expression of this gene might be induced by these conditions in some brain cancers. Addditionally expression of this gene is at a higher level in gliomas compared to medulloblastomas in 3 of the 5 glioma samples in this panel.

Oncology_cell_line_screening_panel_v3.2 Summary: Ag3767 Highest expression of thes gene is detected in a renal cancer SW 839 cell line (CT=27.3). Moderate to low expression of this gene is also seen in number of cancer cell lines derived from melanoma, prostate, osteosarcoma, fibrosarcoma, rhabdomyosarcoma, bladder, pancreatic, Wilm's tumor, T cell leukemia, uterine, cervical, colon, lung and brain cancers. Therefore, therapeutic modulation of this gene or its protein product may be useful in treatment of these cancers.

Panel 4.1D Summary: Ag3767/Ag4935 Two experments with same probe-primer sets are in good agreement with highest expression of this gene seen in resting astrocytes and kidney (CTs=30-32). In addition, moderate to low expression of this gene is also seen in keratinocytes, resting and activated mucoepidermoid NCI-H292, resting and activated lung and dermal fibroblasts. Therefore, therapeutic modulation of this gene or its protein product through the use of antibodies or small molecule drug may be useful in the threatment of psoriasis, asthma, allergies, chronic obstructive pulmonary disease, emphysema and kidney related diseases including lupus erythematosus.

Panel 5 Islet Summary: Ag4935 Highest expression of this gene is seen in islet cells (CT=27.8). Moderate expression of this gene is also seen in adipose, skeletal muscle, and small intestine. Therefore, therapeutic modulation of this gene or its protein product may be useful in the treatment of endocrine/metabolically related diseases, such as obesity and diabetes, especially type II diabetes.

General oncology screening panel_v_{—}2.4 Summary: Ag3767 Highest expression of this gene is detected in a kidney cancer sample (CT=26.7). High expression of this gene is also seen in two more kidney cancer samples. Expression of this gene is higher in cancer compared to normal adjacent kidney samples. Therefore, expression of this gene may be used as diagnostic marker to detect the presence of kidney cancer and also, therapeutic modulation of this gene or its protein product through the use of antibodies or small molecule drug may be useful in the treatment of kidney cancer.

Moderate to low expression of this gene is also seen in metastatic melanoma, prostate and colon cancer. Therefore, therapeutic modulation of this gene or its protein product may be useful in the treament of metastatic melanoma, prostate and colon cancer.

D. CG169230-01: Calcium Modulating Ligand.

Expression of gene CG169230-01 was assessed using the primer-probe set Ag5719, described in Table DA. Results of the RTQ-PCR runs are shown in Tables DB and DC.

TABLE DA
Probe Name Ag5719

| | | | Start | SEQ ID |
|---|---|---|---|---|
| Primers | Sequences | Length | Position | No |
| Forward | 5′-aaaatgggcgaagtcttcac-3′ | 20 | 796 | 42 |
| Probe | TET-5′-cacttttatcttttgtcatgaactgcttga-3′-TAMRA | 30 | 837 | 43 |
| Reverse | 5′-tggtacttcagagccccaat-3′ | 20 | 869 | 44 |

TABLE DB
General_screening_panel_v1.5
Column A - Rel. Exp.(%) Ag5719, Run 245274434

| Tissue Name | A | Tissue Name | A |
|---|---|---|---|
| Adipose | 14.8 | Renal ca. TK-10 | 20.0 |
| Melanoma* Hs688(A).T | 26.2 | Bladder | 16.4 |
| Melanoma* Hs688(B).T | 29.7 | Gastric ca. (liver met.) NCI-N87 | 19.9 |
| Melanoma* M14 | 27.5 | Gastric ca. KATO III | 29.3 |
| Melanoma* LOXIMVI | 24.3 | Colon ca. SW-948 | 7.9 |
| Melanoma* SK-MEL-5 | 20.3 | Colon ca. SW480 | 93.3 |
| Squamous cell carcinoma | 8.1 | Colon ca.* (SW480 met) SW620 | 39.0 |
| SCC-4 | | | |
| Testis Pool | 21.2 | Colon ca. HT29 | 24.1 |
| Prostate ca.* (bone | 9.1 | Colon ca. HCT-116 | 39.2 |
| met) PC-3 | | | |
| Prostate Pool | 7.0 | Colon ca. CaCo-2 | 21.9 |
| Placenta | 2.7 | Colon cancer tissue | 22.7 |
| Uterus Pool | 15.2 | Colon ca. SW1116 | 9.2 |
| Ovarian ca. OVCAR-3 | 20.2 | Colon ca. Colo-205 | 8.3 |
| Ovarian ca. SK-OV-3 | 30.4 | Colon ca. SW-48 | 12.5 |
| Ovarian ca. OVCAR-4 | 21.2 | Colon Pool | 26.4 |
| Ovarian ca. OVCAR-5 | 79.6 | Small Intestine Pool | 25.9 |
| Ovarian ca. IGROV-1 | 41.2 | Stomach Pool | 16.7 |
| Ovarian ca. OVCAR-8 | 49.0 | Bone Marrow Pool | 9.6 |
| Ovary | 15.0 | Fetal Heart | 4.5 |
| Breast ca. MCF-7 | 44.8 | Heart Pool | 8.8 |
| Breast ca. MDA-MB-231 | 50.3 | Lymph Node Pool | 21.3 |
| Breast ca. BT 549 | 100.0 | Fetal Skeletal Muscle | 7.1 |
| Breast ca. T47D | 13.3 | Skeletal Muscle Pool | 21.5 |
| Breast ca. MDA-N | 20.0 | Spleen Pool | 9.9 |
| Breast Pool | 20.6 | Thymus Pool | 17.1 |
| Trachea | 9.8 | CNS cancer (glio/astro) | 61.1 |
| | | U87-MG | |
| Lung | 25.5 | CNS cancer (glio/astro) | 34.9 |
| | | U-118-MG | |
| Fetal Lung | 17.2 | CNS cancer (neuro; met) | 35.4 |
| | | SK-N-AS | |
| Lung ca. NCI-N417 | 13.3 | CNS cancer (astro) SF-539 | 18.6 |
| Lung ca. LX-1 | 58.6 | CNS cancer (astro) SNB-75 | 77.4 |
| Lung ca. NCI-H146 | 8.1 | CNS cancer (glio) SNB-19 | 37.4 |
| Lung ca. SHP-77 | 19.1 | CNS cancer (glio) SF-295 | 69.7 |
| Lung ca. A549 | 32.3 | Brain (Amygdala) Pool | 18.9 |
| Lung ca. NCI-H526 | 6.5 | Brain (cerebellum) | 43.2 |
| Lung ca. NCI-H23 | 61.1 | Brain (fetal) | 29.7 |
| Lung ca. NCI-H460 | 27.0 | Brain (Hippocampus) Pool | 18.7 |
| Lung ca. HOP-62 | 33.9 | Cerebral Cortex Pool | 20.6 |
| Lung ca. NCI-H522 | 74.2 | Brain (Substantia nigra) Pool | 15.2 |
| Liver | 0.3 | Brain (Thalamus) Pool | 32.1 |
| Fetal Liver | 5.6 | Brain (whole) | 17.6 |
| Liver ca. HepG2 | 18.0 | Spinal Cord Pool | 18.6 |
| Kidney Pool | 55.9 | Adrenal Gland | 9.3 |
| Fetal Kidney | 14.6 | Pituitary gland Pool | 5.1 |
| Renal ca. 786-0 | 20.0 | Salivary Gland | 6.4 |
| Renal ca. A498 | 12.9 | Thyroid (female) | 8.8 |
| Renal ca. ACHN | 16.3 | Pancreatic ca. CAPAN2 | 25.0 |
| Renal ca. UO-31 | 22.4 | Pancreas Pool | 14.5 |

TABLE DC
Panel 5 Islet
Column A—Rel. Exp. (%)Ag5719, Run 244646640

| Tissue Name | A | Tissue Name | A |
|---|---|---|---|
| 97457_Patient-02go_adipose | 16.4 | 94709_Donor 2 AM - A_adipose | 23.2 |
| 97476_Patient-07sk_skeletal muscle | 18.0 | 94710_Donor 2 AM - B_adipose | 9.2 |
| 97477_Patient-07ut_uterus | 12.9 | 94711_Donor 2 AM - C_adipose | 6.4 |
| 97478_Patient-07pl_placenta | 6.3 | 94712_Donor 2 AD - A_adipose | 27.5 |
| 99167_Bayer Patient 1 | 100.0 | 94713_Donor 2 AD - B_adipose | 23.0 |
| 97482_Patient-08ut_uterus | 4.9 | 94714_Donor 2 AD - C_adipose | 29.9 |
| 97483_Patient-08pl_placenta | 3.4 | 94742_Donor 3 U - A_Mesenchymal | 7.2 |
| | | Stem Cells | |
| 97486_Patient-09sk_skeletal muscle | 2.9 | 94743_Donor 3 U - B_Mesenchymal | 18.4 |
| | | Stem Cells | |
| 97487_Patient-09ut_uterus | 6.3 | 94730_Donor 3 AM - A_adipose | 21.6 |
| 97488_Patient-09pl_placenta | 2.8 | 94731_Donor 3 AM - B_adipose | 9.0 |
| 97492_Patient-10ut_uterus | 5.4 | 94732_Donor 3 AM - C_adipose | 8.7 |
| 97493_Patient-10pl_placenta | 7.1 | 94733_Donor 3 AD - A_adipose | 53.6 |
| 97495_Patient-11go_adipose | 3.9 | 94734_Donor 3 AD - B_adipose | 5.8 |
| 97496_Patient-11sk_skeletal muscle | 6.0 | 94735_Donor 3 AD - C_adipose | 12.4 |
| 97497_Patient-11ut_uterus | 11.3 | 77138_Liver_HepG2untreated | 11.4 |
| 97498_Patient-11pl_placenta | 5.5 | 73556_Heart_Cardiac stromal cells | 3.8 |
| | | (primary) | |
| 97500_Patient-12go_adipose | 16.3 | 81735_Small Intestine | 13.5 |
| 97501_Patient-12sk_skeletal muscle | 27.2 | 72409_Kidney_Proximal Convoluted | 2.3 |
| | | Tubule | |
| 97502_Patient-12ut_uterus | 18.4 | 82685_Small intestine_Duodenum | 8.9 |
| 97503_Patient-12pl_placenta | 5.5 | 90650_Adrenal_Adrenocortical adenoma | 7.4 |
| 94721_Donor 2 U - | 19.8 | 72410_Kidney_HRCE | 13.5 |
| A_Mesenchymal Stem Cells | | | |
| 94722_Donor 2 U - | 26.4 | 72411_Kidney_HRE | 12.8 |
| B_Mesenchymal Stem Cells | | | |
| 94723_Donor 2 U - | 44.4 | 73139_Uterus_Uterine smooth muscle | 3.1 |
| C_Mesenchymal Stem Cells | | cells | |

General_screening_panel_v1.5 Summary: Ag5719 Highest expression of this gene is detected in breast cancer BT 549 cell line (CT=27.8). This gene codes for calcium modulating ligand (CAMLG). The CAMLG protein is an integral membrane protein that functions on the endoplasmic reticulum membrane to release calcium, resulting in the activation of NF-AT during T-cell receptor signaling (von Bulow, G. and Bram R. J. (1997) Science 278, 138-141). There is also evidence to suggest that CAMLG may also be plasma membrane localized. It has been shown to interact with a surface protein on B-cells, TAC1 to induce signaling. As a plasma membrane protein, CAMLG may regulate calcium entry from outside the cell to increase the intracellular calcium level. Using PathCalling techniques at Curagen it has been shown that CAMLG intereacts with adiponectin and two agouti proteins. This interaction suggests a very interesting role for CAMLG protein in energy metabolism. CAMLG may participate in mobilizing extracellular calcium. From a very large number of animal studies it is known that an increase in intracellular calcium enhances lipid biosynthesis and adipocyte differentiation and inhibits lipid breakdown, leading to obesity and insulin resistance. In this scenario, CAMLG may deliver a pro-obesity signal by mobilizing extracellular calcium in response to specific stimuli. Interaction of CAMLG with agouti and agouti related protein might be significant in this respect because the latter proteins are known to induce obesity when over expressed. Significantly, adiponectin also interacts with CAMLG, and based on the known function of adiponectin in lipid metabolism described above, it is likely that adiponectin may interfere with CAMLG function. It may do so by two mechanisms, by interfering with the binding of agouti and agouti-related protein, or by directly inhibiting CAMLG by protein-protein interaction. Therefore, from the interpretation of the interaction analysis, along with the expression data, blocking CAMLG function on the membrane surface through the use of antibodies, peptides or small molecule drug may be an effective therapeutic intervention against diseases such as obesity and diabetes and other diseases that involves deregulation of calcium signaling.

Panel 5 Islet Summary: Ag5719 Highest expression of this gene is detected in islet cells (CT=30.3). This gene shows a wide spread expression in this panel, which is in agreement with the expression profile in General_screening_panel_v1.5 and also suggests a role for the gene product in cell survival and proliferation.

### Example D

Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV1b SNP Data:

Four polymorphic variant of NOV1b have been identified and are shown in Table D1.

TABLE D1
SNP Variants for CG103362-02.

| | Nucleotides | | | Amino Acids | | |
|---|---|---|---|---|---|---|
| Variant | Position | Initial | Modified | Position | Initial | Modified |
| 13382169 | 1748 | C | T | 479 | Ala | Ala |
| 13382162 | 2098 | T | C | 596 | Leu | Pro |
| 13382163 | 2227 | A | G | 639 | Glu | Gly |
| 13382168 | 2333 | C | T | 674 | Pro | Pro |

NOV5a SNP Data:

One polymorphic variant of NOV5a has been identified and are shown in Table D2.

TABLE D2
SNP Variants for CG169422-01.

| | Nucleotides | | | Amino Acids | | |
|---|---|---|---|---|---|---|
| Variant | Position | Initial | Modified | Position | Initial | Modified |
| 13382151 | 83 | A | G | 28 | Glu | Gly |

### Example E

Analysis of Novel Interactions in the Obesity Pathway

The present invention discloses novel associations of proteins and polypeptides and the nucleic acids that encode them, as identified in a yeast-2-hybrid screen using a cDNA library or one-by-one matrix reactions as described in Example B.3. The proteins and related proteins that are similar to them are encoded by a cDNA and/or by genomic DNA and were identified in some cases by CuraGen Corporation.

In the current invention, protein interactions may include the interaction of a protein fragment with full-length protein, a protein fragment with another protein fragment, or full-length proteins with each other. The protein interactions disclosed in the present invention may also represent significant discoveries of functional importance to specific diseases or pathological conditions in which novel proteins are found to be components of known pathways, known proteins are found to be components of novel pathways, or novel proteins are found to be components of novel pathways.

The adiponectin (APM1) and calcium modulating ligand (CAMLG) protein(s), and protein family(ies), its interactors and any variants, thereof, are suitable as targets for antibody therapeutics, protein drugs, and/or targets for small molecule drugs. As such, the presence of these complexes and pathways and their dysregulation may be used as a marker or as a diagnostic target for identifying specific pathological states, as targets for therapeutic intervention, in screens of small molecule compounds and/or pharmaceuticals, or for use in cellular or animal models. Thus, the current disclosure includes as an embodiment of the current invention, the cloned nucleic acid sequences, vectors, transfected and/or transformed cell lines, animal models, recombinantly expressed and/or endogenously expressed protein.

The compositions of the present invention will have efficacy for treatment of patients suffering from: cancer; inflammation and autoimmune disorders including Crohn's disease, IBD, allergies, rheumatoid and osteoarthritis, inflammatory skin disorders, allergies, blood disorders; colon cancer, leukemia AIDS; metabolic disorders including diabetes and obesity; pancreatic disorders including pancreatic insufficiency and cancer; and prostate disorders including prostate cancer and other diseases, disorders and conditions of the like.

Summary

In an important aspect, the present invention provides a method of identifying novel proteins, protein interactions, complexes, and/or pathways that are candidates for therapeutic intervention in treating a disease, pathology, abnormal state or condition through the targeting of an entity, which has a specific association with the disease. Use of the discovery includes:

1) use as the basis for a diagnostic or therapeutic intervention for a disease or pathological condition, a protein interaction pair, a complex, collection of interactions, or a pathway that elucidates a previously unappreciated function or biological context for a protein.

2) use of a protein or protein complex as affinity reagent(s) (e.g. as in co-immunoprecipitation or affinity chromatography) as a means of purification or for the identification of the presence of another protein component of the interaction.

3) use for monitoring the formation of an interaction pair or complex as an indicator of a drug's effect, as in the screen of a library of compounds, to identify a particular cellular condition or state.

4) use for the modulation of one component of the pathway in order to elicit changes in the activity or expression of downstream interactors or genes, resulting in the alteration of a particular phenotype.

5) use of compounds, such as those identified in a high throughput screen, to perturb or promote the protein interactions themselves.

6) use, in the case of enzyme/substrate interactions, for monitoring changes in the enzymatic activity and/or generation of the modified substrate as an indicator, such as in a high throughput screen of compounds.

Overview of the Overall Interaction

Adiponectin (APM1) also known as the most abundant adipose gene transcript plays a central role in lipid metabolism. The primary function of adiponectin is to induce muscle cells to burn fat (Yamauchi, K. J. et al. (2001) Nat Med 7, 941-946; Havel, P. J. (2002) Curr. Opin. Lipidol. 13, 51-59). The Calcium modulating ligand (CAMLG) functions in increasing intracellular calcium level by allowing entry of calcium from intracellular and extracellular sources (Gotz-Ulrich V. B. and Bram, R. J. (1997) Science 278, 138-141). Intracellular calcium signaling in adipocytes leads to lipogenesis, inhibition of lipolysis and differentiation of adipocytes leading to obesity (Shi H, Dirienzo, D. and Zemel M. B.(2001) FASEB J.15, 291-293). In line with the known functions of adiponectin in lipid metabolism, the inventors have identified a novel function of adiponectin to inhibit CAMLG signaling on the membrane surface to prevent intracellular calcium build up. A decrease in the intracellular calcium level in adipocytes and muscle cells initiates lipid breakdown and fatty acid metabolism. Therefore, the therapeutic intervention of CAMLG function on the membrane may prevent obesity and diabetes and complications associated with these diseases.

The adiponectin (APM1) gene is the most abundant gene transcript in adipose cells that regulates lipid metabolism in our body. Identification and characterization of the APM1 is described above (see CG127779-02, NOV2a, SEQ ID NOs: 5 and 6). It's primary role is to induce lipid catabolism in cells by inducing the expression of genes encoding CD36 (fatty acid transporter), acylcoA oxidase and uncoupling protein-2 (Saltiel, A. R.(2001) Nature Medicine 7, 887-888). This leads to an enhancement in fatty acid transport, fatty acid combustion, and energy dissipation. The net effect of adiponectin on cells is to decrease the accumulation of lipids in the body. In addition, by increasing fatty acid metabolism adiponectin may improve insulin receptor signaling by removing the attenuating effect of lipid on insulin-induced PI3-kinase activity. It has been shown that injection of adiponectin in leptin deficient Ob/Ob mice dramatically normalizes blood sugar level without affecting the hyperinsulinemic condition in the mice suggesting that the mechanism of action of adiponectin may not involve the sensitization of the cells to the effect of insulin (Berg, A. H. et al. (2001) Nature Medicine 7, 947-953). The synthesis and secretion of Adiponectin is increased by activation of the nuclear receptor PPAR-γ. The mechanism of action of adiponectin remains poorly understood.

The agouti-related protein (AGRP) is upregulated in obese and diabetic mice and stimulates feeding leading to obesity. Identification and characterization of AGRP is described above (see CG169440-01, Agouti related protein, NOV6a, SEQ ID NOs: 25 and 26). It is a 132aa protein that is 25% identical to agouti and is expressed in hypothalamus where it antagonizes the melanocortin receptor signaling. AGRP is believed to be the natural ligand for melanocortin-4 receptor. A polymorphism in the promoter region of AGRP leading to a change from C38 to T is associated with a decrease in promoter activity (Mayfield, D. K. et al. (2001) Biochem. Biophys. Res. Commun. 287, 568-573). Ubiquitous expression of AGRP causes obesity.

The agouti signaling protein (ASIP) is the human homologue of the agouti gene and is 85% identical to the mouse gene. Identification and characterization of ASIP is described above (see, CG169422-01, Agouti signaling protein NOV5a, SEQ ID NOs: 23 and 24) Unlike the mouse gene, human agouti is expressed in the adipose tissue and plays a role in adipocyte metabolism and lipid storage by a calcium-dependent signaling pathway (Xue, B. et al. (1998) FASEB J. 12, 1391-1396). It has also been shown to stimulate the expression of fatty acid synthase leading to fatty acid and triglyceride synthesis. Ectopic expression of ASIP protein leads to obesity and hyperinsulinemia.

The Calcium modulating ligand (CAMLG) protein is an integral membrane protein that functions on the endoplasmic reticulum membrane to release calcium, resulting in the activation of NF-AT during T-cell receptor signaling (von Bulow, G. and Bram R. J. (1997) Science 278, 138-141). Identification and characterization of CAMLG is described above (see CG169230-01 Calcium modulating ligand (CAMLG), NOV4a, SEQ ID NOs: 21 and 22). There is also evidence to suggest that CAMLG may also be plasma membrane localized. It has been shown to interact with a surface protein on B-cells, TAC1 to induce signaling. As a plasma membrane protein, CAMLG may regulate calcium entry from outside the cell to increase the intracellular calcium level. This effect may be mediated by protein-protein interaction between CAMLG protein and proteins present in the extracellular space or on the membrane or inside the cell.

The interaction between CAMLG with two agouti proteins on one hand, and adiponectin on the other, reconstitutes a novel signaling cascade that may directly impact on lipid metabolism as shown in Table 5. By increasing the intracellular calcium buildup in adipocytes as a result of its interaction with ASIP and AGRP proteins, CAMLG protein is likely to deliver signals leading to obesity. However, to counteract the effect of CAMLG, adiponectin may either antagonize the binding between agouti proteins with CAMLG or may inhibit the activity of CAMLG protein itself and prevent the mobilization of extracellular calcium. In either situation the net effect of adiponectin will be to reverse the cellular metabolism towards lipid catabolism preventing buildup of fat in adipocytes and muscles. The expression analysis of the interacting proteins by RTQ-PCR described above indicates that the proteins are expressed in similar tissues under similar diseased conditions, providing further evidence that their interaction may be biologically significant.

Based on the analysis of the interactions, a scheme has been proposed describing how adiponectin and CAMLG may function to regulate lipid metabolism as shown in Table 5. Adiponectin is known to enhance fatty acid metabolism in muscles and adipose tissues. It has been hypothesized to function as a regulator of energy metabolism based on the demonstrations that its levels decreases significantly during high fat feeding of mice. Further, administration of purified adiponectin in leptin deficient ob/ob mice causes normalization of blood sugar. Further more, in diabetic db/db mice injection of adiponectin reduced insulin resistance and improved glucose tolerance. The mechanism of adiponectin action in regulating various aspects of energy metabolism is not understood.

The interaction of adiponectin with CAMLG and the interaction of CAMLG with two agouti proteins suggest a very interesting and unsuspected role for CAMLG protein in energy metabolism. CAMLG protein is known to increase intracellular calcium concentration by mobilizing calcium from intracellular stores such as from the lumen of the endoplasmic reticulum following T-cell receptor activation. Based on the observation that CAMLG interacts with three extracellular proteins, it is speculated that CAMLG may also be on the plasma membrane and may participate in a similar role, of mobilizing extracellular calcium. From a very large number of animal studies it is known that an increase in intracellular calcium enhances lipid biosynthesis and adipocyte differentiation and inhibits lipid breakdown, leading to obesity and insulin resistance. In this scenario, CAMLG may deliver a pro-obesity signal by mobilizing extracellular calcium in response to specific stimuli. Interaction of CAMLG with agouti and agouti related protein might be significant in this respect because the latter proteins are known to induce obesity when over expressed. Significantly, adiponectin also interacts with CAMLG, and based on the known function of adiponectin in lipid metabolism described above, it is likely that adiponectin may interfere with CAMLG function. It may do so by two mechanisms, by interfering with the binding of agouti and agouti-related protein, or by directly inhibiting CAMLG by protein-protein interaction as shown in Table 5.

Therefore, from the interpretation of the interaction analysis, along with the expression data and literature information, it is hypothesized that blocking CAMLG function on the membrane surface may be an effective therapeutic intervention against diseases such as obesity and diabetes and other diseases that involves deregulation of calcium signaling. A candidate therapeutic agent will be an antibody against CAMLG protein or it's c-terminal extracellular domain. Other candidates such as peptides or small molecules that block CAMLG function may also be useful.

Methods of Identifying the Nucleic Acids and Proteins Involved in the Interactions.

Interaction protein pairs are added to CuraGen's PathCalling™ (described in Example B. 3) Protein Interaction Database. This database allows for the discovery of novel pharmaceutical drug targets by virtue of their interactions and/or presence in pathologically related signaling pathways. Protein interactions are subsequently analyzed using bioinformatic tools within GeneScape™, which provides a means of visualization of binary protein interactions, protein complex formation, as well as complete cellular signaling pathways. Specifically, the sequences, which encode proteins APM1, CAMLG, AGRP, and ASIP were found to interact and may result in the formation of a protein complex, or may constitute a series of complexes, which form in order to propagate a cellular signal, which is physiologically relevant to a disease pathology. The specific interactions, which constitute the specific complexes, may also be useful for therapeutic intervention through the use of recombinant protein or antibody therapies, small molecule drugs, or gene therapy approaches. Protein interactions, which are identified through the mining of the PathCalling™ database, can be screened in vitro and in vivo to provide expression, functional, biochemical, and phenotypic information. Assays may be used alone or in conjunction and include, but are not limited to the following technologies; RTQ-PCR, Transfection of recombinant proteins, Co-immunoprecipitation and mass spectrometry, FRET, Affinity Chromatography, Immunohistochemisty or Immunocytochemistry, gene CHIP hybridizations, antisense (i.e. knock-down, knock-up), GeneCalling experiments, and/or biochemical assays (phosphorylation, dephosphorylation, protease, etc . . . ).

Uses of the Compositions.

Small Molecule Screening

In an aspect of the invention, a method for identifying a pharmaceutical agent for treating a disease, pathology, or an abnormal state or condition is provided. The method includes the steps of:

(a) identifying a candidate therapeutic agent for treating said disease, pathology, or abnormal state or condition through the use of biochemical and cellular assays, and animal models.

(b) contacting a biological sample associated with the disease, pathology, or abnormal state or condition with the candidate therapeutic agent;

(c) determining whether the candidate induces an effect on the biological sample associated with a therapeutic response therein; and

(d) identifying a candidate exerting such an effect as a pharmaceutical agent.

In significant embodiments of the second method, the biological sample includes a cell, a tissue or organ, or is a nonhuman mammal.

The present invention is based on the identification of biological macromolecules differentially modulated in a pathologic state, disease, or an abnormal condition or state. Among the pathologies or diseases of present interest include metabolic diseases including those related to endocrinological disorders, cancer, various tumors and neoplasias, inflammatory disorders, central nervous system disorders, and similar abnormal conditions or states. Important metabolic disorders with which the biological macromolecules are associated include diabetes mellitus, especially obesity and Type II diabetes. It is believed that obesity predisposes a subject to Type II diabetes. In very significant embodiments of the present invention, the biological macromolecules implicated in these pathologies and conditions are proteins and polypeptides, and in such cases the present invention is related as well to the nucleic acids that encode them. Methods that may be employed to identify relevant biological macromolecules include any procedures that detect differential expression of nucleic acids encoding proteins and polypeptides associated with the disorder, as well as procedures that detect the respective proteins and polypeptides themselves. Significant methods that have been employed by the present inventors, include GeneCalling® technology and SeqCalling™ technology, disclosed respectively, in U.S. Pat. No. 5,871,697, and in U.S. Ser. No. 09/417,386, filed Oct. 13, 1999, each of which is incorporated herein by reference in its entirety. GeneCalling® is also described in Shimkets, et al., “Gene expression analysis by transcript profiling coupled to a gene database query” Nature Biotechnology 17:198-803 (1999).

The present invention identifies a set of proteins and polypeptides, including naturally occurring polypeptides, precursor forms or proproteins, or mature forms of the polypeptides or proteins, which are implicated as targets for therapeutic agents in the treatment of various diseases, pathologies, abnormal states and conditions. A target may be employed in any of a variety of screening methodologies in order to identify candidate therapeutic agents which interact with the target and in so doing exert a desired or favorable effect. The candidate therapeutic agent is identified by screening a large collection of substances or compounds in an important embodiment of the invention. Such a collection may comprise a combinatorial library of substances or compounds in which, in at least one subset of substances or compounds, the individual members are related to each other by simple structural variations based on a particular canonical or basic chemical structure. The variations may include, by way of nonlimiting example, changes in length or identity of a basic framework of bonded atoms; changes in number, composition and disposition of ringed structures, bridge structures, alicyclic rings, and aromatic rings; and changes in pendent or substituents atoms or groups that are bonded at particular positions to the basic framework of bonded atoms or to the ringed structures, the bridge structures, the alicyclic structures, or the aromatic structures.

A polypeptide, protein complex, or signaling pathway described herein, and that serves as a target in the screening procedure, includes the product of a naturally occurring polypeptide or precursor form or proprotein. The naturally occurring polypeptide, precursor or proprotein includes, e.g., the full-length gene product, encoded by the corresponding gene. The naturally occurring polypeptide also includes the polypeptide, precursor or proprotein encoded by an open reading frame described herein. A “mature” form of a polypeptide or protein arises as a result of one or more naturally occurring processing steps as they may occur within the cell, including a host cell. The processing steps occur as the gene product arises, e.g., via cleavage of the amino-terminal methionine residue encoded by the initiation codon of an open reading frame, or the proteolytic cleavage of a signal peptide or leader sequence. Thus, a mature form arising from a precursor polypeptide or protein that has residues 1 to N, where residue 1 is the N-terminal methionine, would have residues 2 through N remaining. Alternatively, a mature form arising from a precursor polypeptide or protein having residues 1 to N, in which an amino-terminal signal sequence from residue 1 to residue M is cleaved, includes the residues from residue M+1 to residue N remaining. A “mature” form of a polypeptide or protein may also arise from non-proteolytic post-translational modification. Such non-proteolytic processes include, e.g., glycosylation, myristylation or phosphorylation. In general, a mature polypeptide or protein may result from the operation of only one of these processes, or the combination of any of them.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

As used herein, “protein complex” is used to describe a composition, which includes two or more interacting proteins whose interaction is stable and critical for their function. In many cases protein complexes are identified by their presence in closed geometrical structures or polygons, such as triangles or squares. For example, protein A interacts with protein B, which interacts with protein C, which interacts with protein A (i.e. A-B-C-A). Additionally, a complex could constitute a functional precursor in a manner similar to the hetero-trimeric G-protein paradigm. The protein complex identified therefore constitutes a discrete biologically functional entity and significant utility claims are based on this embodiment.

As used herein, “protein pathway” is used to describe a collection of protein interactions, which are involved in the propagation or transduction of cellular signals or constitute a portion of the signal therein. “Protein pathway” also includes collections of interactions, which may be composed of transient protein interactions, stable protein interactions or a combination of both.

As used herein, the term “perturb” is defined as the ability of a chemical compound or biological agent (e.g. a peptide, a protein, or nucleotide), to antagonize, inhibit, or abolish a protein interaction. The perturbation of protein-protein interactions may be achieved by several plausible mechanisms. For example, the chemical compound or biological agent may bind selectively to the interaction “active site” or “binding site”, thereby blocking the interaction from occurring. Additionally, a chemical compound or biological agent may bind to another site on the target protein resulting in a conformational or structural change, which prevents the interaction from taking place. Alternatively, a compound or biological agent could result in the alteration of a posttranslational modification, which is critical for mediating the protein-protein interaction, thereby disrupting the protein complex.

As used herein, a “chemical compound” relates to a composition that includes at least one chemical substance, or to one or more substances extracted, for example, from a common natural source. A chemical substance may be the product of a defined synthetic procedure. Such a synthesized compound is understood herein to have defined properties in terms of molecular formula, molecular structure relating the association of bonded atoms to each other, physical properties such as chromatographic or spectroscopic characterizations, and the like. A substance extracted from a natural source is advantageously analyzed by chemical and physical methods in order to provide a representation of its defined properties, including its molecular formula, molecular structure relating the association of bonded atoms to each other, physical properties such as chromatographic or spectroscopic characterizations, and the like.

As used herein, a “candidate therapeutic agent” is a chemical compound that includes at least one chemical substance, wherein at least one such substance has been shown to bind to a target biopolymer. In important embodiments of the invention, the target biopolymer is a protein or polypeptide, a nucleic acid, a polysaccharide or proteoglycan, or a lipid such as a complex lipid. The method of identifying compounds that bind to the target effectively eliminates compounds with little or no binding affinity, thereby increasing the potential that the identified chemical compound may have beneficial therapeutic applications. In cases where the chemical compound is a mixture of more than one chemical substance, subsequent screening procedures may be carried out to identify the particular chemical substance in the mixture that is the binding compound, and that is to be identified as a candidate therapeutic agent.

As used herein, a “pharmaceutical agent” is provided by screening a candidate therapeutic agent using models for a disease state or pathology in order to identify a candidate exerting a desired or beneficial therapeutic effect with relation to the disease or pathology. Such a candidate that successfully provides such an effect is termed a pharmaceutical agent herein. Nonlimiting examples of model systems that may be used in such screens include particular cell lines, cultured cells, tissue preparations, whole tissues, organ preparations, intact organs, and nonhuman mammals. Screens employing at least one system, and preferably more than one system, may be employed in order to identify a pharmaceutical agent. Any pharmaceutical agent so identified may be pursued in further investigation using human subjects.

The current disclosure presents many significant and plausible uses for the invention. However, this disclosure is not meant to limit the scope of application of the invention to other methods or technologies for which it may be well suited.

In a specific embodiment, the interaction of adiponectin (APM1) with calcium modulating ligand (CAMLG) reveals a novel pathway by which APM1 may induce lipid breakdown in muscle cells and adipocytes. The interaction between agouti signaling protein (ASIP) with CAMLG suggests a novel mechanism by which ASIP may increase calcium level in cells. In both instances CAMLG appears to be the target of proteins that promote obesity as well as that counter obesity, as schematically shown in Table 1 and 5. This makes CAMLG a novel target to control obesity and may be useful in potential diagnostic and therapeutic applications. The therapeutic applications may include (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. Similar therapeutic interventions can be envisaged for the interacting proteins in the pathway for controlling metabolic disorders such as obesity, diabetes, and complications associated with these diseases.

The yeast-2-hybrid system was used to identify the interacting proteins disclosed in the present invention. The proteins involved in these interactions likely participate in the same physiological pathway. Because of the significance of these pathways, the present invention provides a list of uses for these proteins and/or the DNA encoding these proteins, as a basis for developing therapeutic and diagnostic tools.

Descriptions of the GeneCalling Discovery Studies

MB.04

A large number of mouse strains have been identified that differ in body mass and composition. The AKR and NZB strains are obese, the SWR, C57L and C57BL/6 strains are of average weight whereas the SM/J and Cast/Ei strains are lean. Understanding the gene expression differences in the major metabolic tissues from these strains will elucidate the pathophysiologic basis for obesity. These specific strains of rat were chosen for differential gene expression analysis because quantitative trait loci (QTL) for body weight and related traits had been reported in published genetic studies. Tissues included whole brain, skeletal muscle, visceral adipose, and liver.

BP24.02

The predominant cause for obesity in clinical populations is excess caloric intake. This so-called diet-induced obesity (DIO) is mimicked in animal models by feeding high fat diets of greater than 40% fat content. The DIO study was established to identify the gene expression changes contributing to the development and progression of diet-induced obesity. In addition, the study design seeks to identify the factors that lead to the ability of certain individuals to resist the effects of a high fat diet and thereby prevent obesity. The sample groups for the study had body weights +1 S.D., +4 S.D. and +7 S.D. of the chow-fed controls (below). In addition, the biochemical profile of the +7 S.D. mice revealed a further stratification of these animals into mice that retained a normal glycemic profile in spite of obesity and mice that demonstrated hyperglycemia. Tissues examined included hypothalamus, brainstem, liver, retroperitoneal white adipose tissue (WAT), epididymal WAT, brown adipose tissue (BAT), gastrocnemius muscle (fast twitch skeletal muscle) and soleus muscle (slow twitch skeletal muscle). The differential gene expression profiles for these tissues should reveal genes and pathways that can be used as therapeutic targets for obesity.

GeneCalling Data:

A gene fragment of the mouse Adiponectin was found in MB.04 study to be up-regulated by 2 fold in the adipose tissue of genetically obese mouse (AKR strain) relative to normal mouse (SWR strain) using CuraGen's GeneCalling™ method of differential gene expression. The GeneCalling™ method makes a comparison between experimental samples in the amount of each cDNA fragment generated by digestion with a unique pair of restriction endonucleases, after linker-adaptor ligation, PCR amplification and chromatographic separation. Computer analysis is employed to assign potential identity to the gene fragment. A gene fragment of the mouse Adiponectin was also found in BP24.02 study to be down-regulated several fold in the adipose tissue of diet induced obese animals relative to learner animals on the same diet. Differential expressions of adiponectin in different animal models of obesity together with literature data strongly suggest the role of adiponectin in development of obesity.

Tables

Table 1. illustrates a, diagram of novel interactions in the adiponectin (APM1) pathway.

Table 2. illustrates a diagram of amino acid boundaries of proteins involved in the interaction and the source of the cDNA libraries.

Table 3. illustrates a graph of the body weight distribution of mouse strains used in GeneCalling studies

Table 4 illustrates specific primers against mouse adiponectin gene used in competitive PCR experiments

Table 5. illustrates a schematic diagram of the role of adiponectin in preventing obesity and identify calcium modulating ligand as a novel target of therapeutic intervention.

TABLE 1

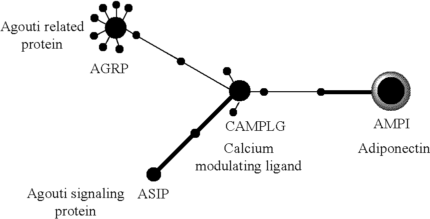

TABLE 2
APM1 adipose most abundant gene transcript 1
CAMLG calcium modulating ligand

| Experiment | Status | # | APM1 | CAMLG |
|---|---|---|---|---|
| PC-Fetal Brain | Frame 1 (+) | 5 | 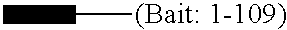 | 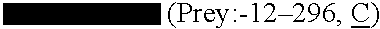 |
| PC-Fetal Brain | Frame 1 (+) | 1 | 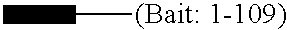 | 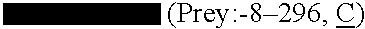 |
| PC-HeLa | Frame 1 (+) | 4 | 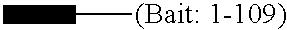 | 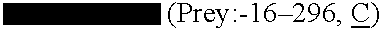 |

AGRP agouti related protein homolog (mouse)
CAMLG calcium modulating ligand

| Experiment | Status | # | AGRP | CAMLG |
|---|---|---|---|---|
| PC-Lung | Frame 1 (+) | 1 | 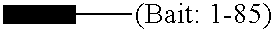 | 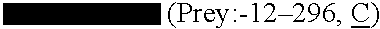 |
| PC-Lung | Frame 1 (+) | 1 | 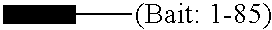 | 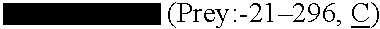 |

ASIP agoul signaling protein, nonagouti homolog (mouse)
CAMLG calcium modulating ligand

| Experiment | Status | # | ASIP | CAMLG |
|---|---|---|---|---|
| PC-HeLa | Frame 1 (+) | 7 | 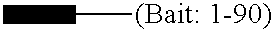 | 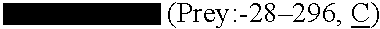 |
| PC-HeLa | Frame 1 (+) | 1 | 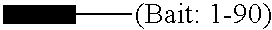 | 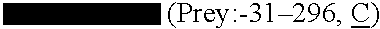 |

[image] TABLE 4
Gene Sequence (fragment from 116 to 453 in bold band size: 338,
specific primers are underlined)

SEQ ID:45

| | |
|---|---|
| 1 | GATTGTCAGT GGATCTGACG ACACCAAAAG GGCTCAGGAT GCTACTGTTG CAAGCTCTCC |
| 61 | TGTTCCTCTT AATCCTGCCC AGTCATGCCG AAGATGACGT TACTACAACT GAAGAGCTAG |
| 121 | CTCCTGCTTT GGTCCCTCCA CCCAAGGGAA CTTGTGCAGG TTGGATGGCA GGCATCCCAG |
| 181 | GACATCCTGG CCACAATGGC ACACCAGGCC GTGATGGCAG AGATGGCACT CCTGGAGAGA |
| 241 | AGGGAGAGAA AGGAGATGCA GGTCTTCTTG GTCCTAAGGG TGAGACAGGA GATGTTGGAA |
| 301 | TGACAGGAGC TGAAGGGCCA CGGGGCTTCC CCGGAACCCC TGGCAGGAAA GGAGAGCCTG |
| 361 | GAGAAGCCGC TTATATGTAT CGCTCAGCGT TCAGTGTGGG GCTGGAGACC CGCGTCACTG |
| 421 | TTCCCAATGT ACCCATTCGC TTTACTAAGA TCTTCTACAA CCAACAGAAT CATTATGACG |
| 481 | GCAGCACTGG CAAGTTCTAC TGCAACATTC CGGGACTCTA CTACTTCTCT TACCACATCA |
| 541 | CGGTGTACAT GAAAGATGTG AAGGTGAGCC TCTTCAAGAA GGACAAGGCC GTTCTCTTCA |
| 601 | CCTAGGACCA GTATCAGGAA AAGAATGTGG ACCAGGCCTC TGGCTCTGTG CTCCTCCATC |
| 661 | TGGAGGTGGG AGACCAAGTC TGGCTCCAGG TGTATGGGGA TGGGGACCAC AATGGACTCT |
| 721 | ATGCAGATAA CGTCAACGAC TCTACATTTA CTGGCTTTCT TCTCTACCAT GATACCAACT |
| 781 | GACTGCAACT ACCCATAGCC CATACACCAG GAGAATCATG GAACAGTCCA CACACTTTCA |
| 841 | GCTTACTTTG AGAGATTGAT TTTATTGCTT AGTTTGAGAG TCCTGACTAT TATCCACACG |
| 901 | TGTACTCACT TGTTCATTAA ACGACTTTAT AAA |

(gene length is 1215, only region from 1 to 933 shown)

TABLE 5

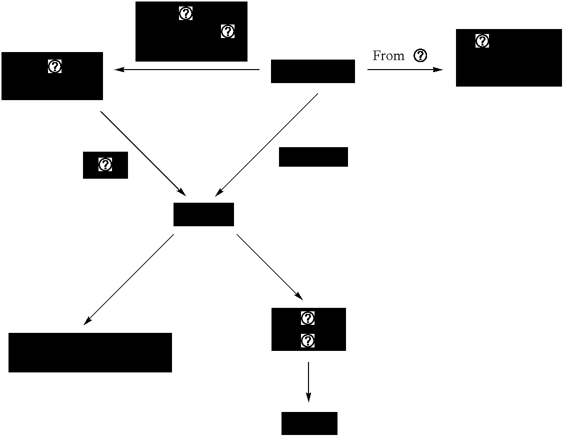

### Example F

Expression of CG92035-03 in Escherichia coli Strain E397.

A 0.387 kb BamHI-XhoI fragment containing the CG92035-03 sequence was subcloned into BamHI-XhoI digested pET28-His (Invitrogen) to generate plasmid 1783. The resulting plasmid 1783 was transformed into E. coli using the standard transformation protocol. The cell pellet and supernatant were harvested 2 h post induction with IPTG and examined for CG92035-03 expression by Western blot (reducing conditions) using an anti-HIS antibody. CG92035-03 is expressed as a 15 kDa protein.

Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO: 2n, wherein n is an integer between 1 and 13.

2. An isolated polypeptide comprising an amino acid sequence selected from the group consisting of SEQ ID NO: 2n, wherein n is an integer between 1 and 13.

3. An isolated polypeptide comprising an amino acid sequence which is at least 95% identical to an amino acid sequence selected from the group consisting of SEQ ID NO: 2n, wherein n is an integer between 1 and 13.

4. An isolated polypeptide, wherein the polypeptide comprises an amino acid sequence comprising one or more conservative substitutions in the amino acid sequence selected from the group consisting of SEQ ID NO: 2n, wherein n is an integer between 1 and 13.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2n, wherein n is an integer between 1 and 13, or a biologically active fragment thereof.

20. An isolated nucleic acid molecule comprising a nucleic acid sequence selected from the group consisting of SEQ ID NO: 2n−1, wherein n is an integer between 1 and 13.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 13.

23. An isolated nucleic acid molecule encoding the mature form of a polypeptide having an amino acid sequence selected from the group consisting of SEQ ID NO: 2n, wherein n is an integer between 1 and 13.

24. An isolated nucleic acid molecule comprising a nucleic acid selected from the group consisting of 2n−1, wherein n is an integer between 1 and 13.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 13, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step
(a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n−1, wherein n is an integer between 1 and 13.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n−1, wherein n is an integer between 1 and 13.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
